(19) United States
(12) Patent Application Publication
(10) Pub. No.: US 2010/0022446 A1
(43) Pub. Date: Jan. 28, 2010

(54) Use of Peptides in Combination with Surgical Intervention for the Treatment of Obesity

(75) Inventors: Birgitte Schjellerup Wulff, Virum (DK); Kirsten Raun, Lyngby (DK); Birgit Sehested Hansen, Stenlose (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/523,197

(22) PCT Filed: Jan. 17, 2008
(86) PCT No.: PCT/EP2008/050516

§ 371 Date: Sep. 3, 2009

(30) Foreign Application Priority Data
Jan. 18, 2007 (DK) PA 2007 00081

(51) Int. Cl. A61K 38/02; A61P 3/04

(52) U.S. Cl. 514/9; 514/2; 514/17

(57) ABSTRACT

The present invention relates to novel peptide compounds which are effective in modulating one or more melanocortin receptor types, to the use of the compounds in therapy, to methods of treatment comprising administration of the compounds to patients in need thereof, and to the use of the compounds in the manufacture of medicaments. The compounds of the invention are of particular interest in relation to the treatment of obesity as well as a variety of diseases or conditions associated with obesity.

### FIELD OF THE INVENTION

The present invention relates, inter alia, to the use of peptides which are selective for one or more melanocortin receptors, and which may exert a prolonged activity, for administration to patients in combination with surgical intervention for the purpose of achieving weight loss or preventing weight gain in the patient.

### BACKGROUND OF THE INVENTION

Obesity is a well known risk factor for the development of many very common diseases such as atherosclerosis, hypertension, type 2 diabetes (non-insulin dependent diabetes mellitus (NIDDM)), dyslipidaemia, coronary heart disease, and osteoarthritis and various malignancies. It also causes considerable problems through reduced motility and decreased quality of life. The incidence of obesity and thereby also these diseases is increasing throughout the entire industrialised world. Only a few pharmacological treatments are available to date, namely Sibutramine (Abbot; acting via serotonergic and noradrenaline mechanisms), Orlistat (Roche Pharm; reducing fat uptake from the gut,) and Acomplia (rimonabant; Sanofi-Aventis; CB1 endocannabinoid receptor antagonist; approved in EU in June 2006). However, due to the important effect of obesity as a risk factor in serious and even fatal and common diseases there is still a need for pharmaceutical compounds useful in the treatment of obesity.

The term obesity implies an excess of adipose tissue. In this context, obesity is best viewed as any degree of excess adiposity that imparts a health risk. The distinction between normal and obese individuals can only be approximated, but the health risk imparted by obesity is probably a continuum with increasing adiposity. However, in the context of the present invention, individuals with a Body Mass Index (BMI=body weight in kilograms divided by the square of the height in meters) above 25 are to be regarded as obese.

Even mild obesity increases the risk for premature death, diabetes, hypertension, atherosclerosis, gallbladder disease and certain types of cancer. In the industrialized western world the prevalence of obesity has increased significantly in the past few decades. Because of the high prevalence of obesity and its health consequences, its treatment should be a high public health priority.

When energy intake exceeds energy expenditure, the excess calories are stored in adipose tissue, and if this net positive balance is prolonged, obesity results, i.e. there are two components to weight balance, and an abnormality on either side (intake or expenditure) can lead to obesity.

Pro-opiomelanocortin (POMC) is the precursor for β-endorphin and melanocortin peptides, including melanocyte stimulating hormone (α-MSH) and adrenocorticotropin (ACTH). POMC is expressed in several peripheral and central tissues including melanocytes, the pituitary, and neurons of the hypothalamus. The POMC precursor is processed differently in different tissues, resulting in the expression of different melanocortin peptides depending on the site of expression. In the anterior lobe of the pituitary, mainly ACTH is produced whereas in the intermediate lobe and the hypothalamic neurons the major peptides are α-MSH, β-MSH, desacetyl-α-MSH and β-endorphin. Several of the melanocortin peptides, including ACTH and α-MSH, have been demonstrated to have appetite-suppressing activity when administered to rats by intracerebroventricular injection [Vergoni et al, European Journal of Pharmacology 179, 347-355 (1990)]. An appetite-suppressing effect is also obtained with the artificial cyclic α-MSH analogue, MT-II.

A family of five melanocortin receptor subtypes has been identified (melanocortin receptor 1-5, also called MC1, MC2, MC3, MC4 and MC5). The MC1, MC2 and MC5 are mainly expressed in peripheral tissues, whereas MC3 and MC4 are mainly centrally expressed; MC3 are, however, also expressed in several peripheral tissues. In addition to being involved in energy homeostasis, MC3 receptors have also been suggested to be involved in several inflammatory diseases. An MC3 agonist could have a positive effect on such diseases, e.g. gouty arthritis. MC5 are mainly peripherally expressed, and have been suggested to be involved in exocrine secretion and in inflammation. MC4 have been shown to be involved in the regulation of body weight and feeding behavior, as MC4 knock-out mice develop obesity [Huzar et al., Cell 88, 131-141 (1997)]. Furthermore, studies of either ectopic central expression of agouti protein (MC1, MC3 and MC4 antagonist) or over-expression of an endogenously occurring MC3 and MC4 antagonist (agouti gene related protein, AGRP) in mouse brain demonstrated that the over-expression of these two antagonists led to the development of obesity [Kleibig et al., PNAS 92, 4728-4732 (1995)]. Moreover, icv injection of a C-terminal fragment of AGRP increases feeding and antagonizes the inhibitory effect of α-MSH on food intake.

In humans, several cases of families with obesity which is presumably due to frame shift mutations in MC4 have been described [see, e.g., Yeo et al., Nature Genetics 20, 111-112 (1998); Vaisse et al., Nature Genetics 20, 113-114 (1998)]. Mutations in the gene encoding the MC4 receptor appear to be the most abundant monogenic cause of obesity [Farooqi et al., New England Journal of Medicine 384, 1085-1095 (2003)]

In conclusion, a MC4 agonist could serve as an anorectic drug and/or energy expenditure increasing drug and be useful in the treatment of obesity or obesity-related diseases, as well as in the treatment of other diseases, disorders or conditions which may be ameliorated by activation of MC4.

MC4 antagonists may be useful for treatment of cachexia or anorexia, and for treatment of waisting in frail elderly patients. Furthermore, MC4 antagonists may be used for treatment of chronic pain, neuropathy and neurogenic inflammation.

A large number of patent applications disclose various classes of non-peptidic small molecules as melanocortin receptor modulators; examples hereof are WO 03/009850, WO 03/007949 and WO 02/081443.

The use of peptides as melanocortin receptor modulators is disclosed in a number of patent documents, e.g. WO 03/006620, U.S. Pat. No. 5731,408 and WO 98/27113. Hadley [Pigment Cell Res. 4, 180-185, (1991)] reports a prolonged effect of specific melanotropic peptides conjugated to fatty acids, the prolongation effected by a transformation of the modulators from being reversibly acting to being irreversibly acting being caused by the conjugated fatty acids.

### SUMMARY OF THE INVENTION

It was found that certain peptide conjugates, identified below, have a high modulating effect on one or more melanocortin receptors, i.e. the MC1, MC2, MC3, MC4 or MC5. The present invention relates to the use of compounds of this type, in combination with surgical intervention intended for the purpose of achieving weight loss or preventing weight gain (i.e. bariatric surgical intervention), in order to achieve greater weight loss or more satisfactory prevention of weight gain in a patient so treated than is achieved by use of one of the two types of treatment (i.e. administration of a compound of the type in question, and surgical intervention, respectively) alone. Among compounds (more particularly compounds acting as melanocortin receptor agonists or antagonists) of the type in question are compounds of the following formula I:

T-A-L-P [I]

wherein

T represents tetrazol-5-yl;

A represents a straight-chain, branched and/or cyclic C₆₋₂₀alkyl, C₆₋₂₀alkenyl or C₆₋₂₀alkynyl which may optionally be substituted with one or more substituents selected from halogen, hydroxy and aryl;

L is a bond or a chemical structure covalently linking A and P; and

P represents a peptide structure comprising at least six α-amino acid residues.

Among compounds of the type in question are compounds having the formula II:

R¹—R²—C(═O)—R³—S¹-Z¹-Z²-Z³-Z⁴-Z⁵-Z⁶-c[X¹—X²—X³-Arg-X⁴—X^{5]—R}⁴ [II]

wherein

R¹ represents tetrazol-5-yl or carboxy;

R² represents a straight-chain, branched and/or cyclic C₆₋₂₀alkyl, C₆₋₂₀alkenyl or C₆₋₂₀alkynyl which may optionally be substituted with one or more substituents selected from halogen, hydroxy and aryl;

R³ is absent or represents —NH—S(═O)₂—(CH₂)₃₋₅—C(═O)— or a peptide fragment comprising one or two amino acid residues and containing at least one carboxy group;

Si is absent or represents a 4-aminobutyric acid residue, Gly, β-Ala, or a glycolether-based structure according to one of the formulas IIIa-IIIg;

—HN—CH₂—CH₂—O—CH₂—CH₂—O—CH₂—C(═O)— [IIIa]

—[HN—CH₂—CH₂—O—CH₂—CH₂—O—CH₂—C(═O)]₂— [IIIb]

—[HN—CH₂—CH₂—O—CH₂—CH₂—O—CH₂—C(═O)]₃₋₅— [IIIc]

—[HN—CH₂—CH₂—O—CH₂—CH₂—O—CH₂—CH₂—NH—C(═O)—CH₂—CH₂—CH₂—C(═O)]₁₋₃— [IIId]

—[HN—CH₂—CH₂—O—CH₂—CH₂—O—CH₂—CH₂—NH—C(═O)—CH₂—O—CH₂—C(═O)]₁₋₃— [IIIe]

—[HN—CH₂—CH₂—O—CH₂—CH₂—O—CH₂—CH₂—O—CH₂—CH₂—O—CH₂—CH₂—C(═O)]₁₋₃— [IIIf]

—HN—CH₂—CH₂]—₂₋₁₂—O—CH₂—C(═O)— [IIIg]

—HN—CH₂—CH₂—[O—CH₂—CH₂]₄₋₁₂—O—CH₂—CH₂—C(═O)— [IIIh]

Z¹ is absent or represents Gly, β-Ala, Ser, D-Ser, Thr, D-Thr, His, D-His, Asn, D-Asn, Gln, D-Gln, Glu, D-Glu, Asp, D-Asp, Ala, D-Ala, Pro, D-Pro, Hyp or D-Hyp;

Z² is absent or represents Gly, β-Ala, Ser, D-Ser, Thr, D-Thr, His, D-His, Asn, D-Asn, Gln, D-Gln, Glu, D-Glu, Asp, D-Asp, Ala, D-Ala, Pro, D-Pro, Hyp or D-Hyp;

Z³ represents Ser, D-Ser, Thr, D-Thr, His, D-His, Asn, D-Asn, Gln, D-Gln, Glu, D-Glu, Asp, D-Asp, Ala, D-Ala, Pro, D-Pro, Hyp or D-Hyp;

Z⁴ represents Gly, Ala, Pro, Hyp, Ser, homoSer, Thr, Tyr, Gln, Asn, 2-PyAla, 3-PyAla, 4-PyAla, His, homoArg, Arg, Lys, Dab, Dap or Orn;

Z⁵ represents Gly, Ala, Pro, Hyp, Ser, homoSer, Thr, Gln, Asn, 2-PyAla, 3-PyAla, 4-PyAla, His, homoArg, Arg, Lys, Dab, Dap or Orn;

Z⁶ represents Ala, D-Ala, Val, D-Val, Leu, D-Leu, Ile, D-Ile, Met, D-Met, Nle or D-Nle;

X¹ represents Glu, Asp, Cys, homoCys, Lys, Orn, Dab or Dap;

X² represents His, Cit, Dab, Dap, Cgl, Cha, Val, Ile, tBuGly, Leu, Tyr, Glu, Ala, Nle, Met, Met(O), Met(O₂), Gln, Gln(alkyl), Gln(aryl), Asn, Asn(alkyl), Asn(aryl), Ser, Thr, Cys, Pro, Hyp, Tic, 2-PyAla, 3-PyAla, 4-PyAla, (2-thienyl)alanine, 3-(thienyl)alanine, (4-thiazolyl)Ala, (2-furyl)alanine, (3-furyl)alanine or Phe, wherein one or more hydrogens on the phenyl moiety of the Phe in question may optionally and independently be substituted by a substituent selected among halogen, hydroxy, alkoxy, nitro, benzoyl, methyl, trifluoromethyl, amino and cyano;

X³ represents D-Phe, wherein one or more hydrogens on the phenyl moiety in D-Phe may optionally and independently be substituted by a substituent selected among halogen, hydroxy, alkoxy, nitro, methyl, trifluoromethyl and cyano;

X⁴ represents Trp, 2-Nal, (3-benzo[b]thienyl)alanine or (S)-2,3,4,9-tetrahydro-1H-β-carboline-3-carboxylic acid;

X⁵ represents Glu, Asp, Cys, homoCys, Lys, Orn, Dab or Dap;

wherein X¹ and X⁵ are joined, rendering the compound of formula II cyclic, either via a disulfide bridge deriving from X¹ and X⁵ both independently being Cys or homoCys, or via an amide bond formed between a carboxylic acid in the side-chain of X¹ and an amino group in the side-chain of X⁵, or between a carboxylic acid in the side-chain of X⁵ and an amino group in the side-chain of X¹;

R⁴ represents OR′ or N(R′)₂, wherein each R′ independently represents hydrogen or represents C₁₋₆alkyl, C₂₋₆alkenyl or C₂₋₆alkynyl which may optionally be substituted with one or more amino or hydroxy;

and pharmaceutically acceptable salts, prodrugs and solvates thereof.

Among other compounds of the type in question are compounds having the formula IVa, IVb or IVc:

R¹—R²—C(═O)—R³—S²-Z⁴-Z⁵-Z⁶-c[X¹—X²—X³-Arg-X⁴—X⁵]R⁴ [IVa]

R¹—R²—C(═O)—R³—S²-Z⁵-Z⁶-c[X¹—X²—X³-Arg-X⁴—X⁵]R⁴ [IVb]

R¹—R²—C(═O)—R³—S²-Z⁶-c[X¹—X²—X³-Arg-X⁴—X⁵]R⁴ [IVc]

wherein

R¹ represents tetrazol-5-yl or carboxy;

R² represents a straight-chain, branched and/or cyclic C₆₋₂₀alkyl, C₆₋₂₀alkenyl or C₆₋₂₀alkynyl which may optionally be substituted with one or more substituents selected from halogen, hydroxyl and aryl;

R³ is absent or represents —NH—S(═O)₂—(CH₂)₃₋₅—C(═O)— or a peptide fragment comprising one or two amino acid residues and containing at least one carboxy group;

S² represents a glycolether-based structure according to one of the formulas IIIa-IIIh;

—HN—CH₂—CH₂—O—CH₂—CH₂—O—CH₂—C(═O)— [IIIa]

—[HN—CH₂—CH₂—O—CH₂—CH₂—O—CH₂—C(═O)]₂— [IIIb]

—[HN—CH₂—CH₂—O—CH₂—CH₂—O—CH₂—C(═O)]₃₋₅— [IIIc]

—[HN—CH₂—CH₂—O—CH₂—CH₂—O—CH₂—CH₂—NH—C(═O)—CH₂—CH₂—CH₂—C(═O)]₁₋₃— [IIId]

—[HN—CH₂—CH₂—O—CH₂—CH₂—O—CH₂—CH₂—NH—C(═O)—CH₂—O—CH₂—C(═O)]₁₋₃— [IIIe]

—[HN—CH₂—CH₂—O—CH₂—CH₂—O—CH₂—CH₂—O—CH₂—CH₂—O—CH₂—CH₂—C(═O)]₁₋₃— [IIIf]

—HN—CH₂—CH₂—[O—CH₂—CH₂]₂₋₁₂—O—CH₂—C(═O)— [IIIg]

—HN—CH₂—CH₂—[O—CH₂—CH₂]₄₋₁₂—O—CH₂—CH₂—C(═O)— [IIIh]

Z⁴ represents Gly, Ala, Pro, Hyp, Ser, homoSer, Thr, Tyr, Gln, Asn, 2-PyAla, 3-PyAla, 4-PyAla, His, homoArg, Arg, Lys, Dab, Dap or Orn;

Z⁵ represents Gly, Ala, Pro, Hyp, Ser, homoSer, Thr, Gln, Asn, 2-PyAla, 3-PyAla, 4-PyAla, His, homoArg, Arg, Lys, Dab, Dap or Orn;

Z⁶ represents Ala, D-Ala, Val, D-Val, Leu, D-Leu, Ile, D-Ile, Met, D-Met, Nle or D-Nle;

X¹ represents Glu, Asp, Cys, homoCys, Lys, Orn, Dab or Dap;

X² represents His, Cit, Dab, Dap, Cgl, Cha, Val, Ile, tBuGly, Leu, Tyr, Glu, Ala, Nle, Met, Met(O), Met(O₂), Gln, Gln(alkyl), Gln(aryl), Asn, Asn(alkyl), Asn(aryl), Ser, Thr, Cys, Pro, Hyp, Tic, 2-PyAla, 3-PyAla, 4-PyAla, (2-thienyl)alanine, 3-(thienyl)alanine, (4-thiazolyl)Ala, (2-furyl)alanine, (3-furyl)alanine or Phe, wherein one or more hydrogens on the phenyl moiety of the Phe in question may optionally and independently be substituted by a substituent selected among halogen, hydroxy, alkoxy, nitro, benzoyl, methyl, trifluoromethyl, amino and cyano;

X³ represents D-Phe, wherein one or more hydrogens on the phenyl moiety in D-Phe may optionally and independently be substituted by a substituent selected among halogen, hydroxy, alkoxy, nitro, methyl, trifluoromethyl and cyano;

X⁴ represents Trp, 2-Nal, (3-benzo[b]thienyl)alanine or (S)-2,3,4,9-tetrahydro-1H-β-carboline-3-carboxylic acid;

X⁵ represents Glu, Asp, Cys, homoCys, Lys, Orn, Dab or Dap;

wherein X¹ and X⁵ are joined, rendering the compound of formula IVa, IVb or IVc cyclic, either via a disulfide bridge deriving from X¹ and X⁵ both independently being Cys or homoCys, or via an amide bond formed between a carboxylic acid in the side-chain of X¹ and an amino group in the side-chain of X⁵, or between a carboxylic acid in the side-chain of X⁵ and an amino group in the side-chain of X¹;

R⁴ represents OR′ or N(R′)₂, wherein each R′ independently represents hydrogen or represents C₁₋₆alkyl, C₂₋₆alkenyl or C₂₋₆alkynyl which may optionally be substituted with one or more amino or hydroxy;

and pharmaceutically acceptable salts, prodrugs and solvates thereof.

### DEFINITIONS

The use of a prefix of the type “C_{x-y}” preceding the name of a radical, such as in C_{x-y}alkyl (e.g. C₆₋₂₀alkyl) is intended to indicate a radical of the designated type having from x to y carbon atoms.

The term “alkyl” as used herein refers to a straight-chain, branched and/or cyclic, saturated monovalent hydrocarbon radical.

The term “alkenyl” as used herein refers to a straight-chain, branched and/or cyclic, mono-valent hydrocarbon radical comprising at least one carbon-carbon double bond.

The term “alkynyl” as used herein refers to a straight-chain, branched and/or cyclic, monovalent hydrocarbon radical comprising at least one carbon-carbon triple bond, and it may optinally also comprise one or more carbon-carbon double bonds.

The term “alkoxy” as used herein is intended to indicate a radical of the formula —OR′, wherein R′ is alkyl as indicated above.

In the present context, the term “aryl” is intended to indicate a carbocyclic aromatic ring radical or a fused aromatic ring system radical wherein at least one of the rings is aromatic. Typical aryl groups include phenyl, biphenylyl, naphthyl, and the like.

The term “halogen” is intended to indicate members of the 7^{th} main group of the periodic table of the elements, which includes fluorine, chlorine, bromine and iodine (corresponding to fluoro, chloro, bromo and iodo substituents, respectively).

The term “tetrazol-5-yl” is intended to indicate 1H-tetrazol-5-yl or 2H-tetrazol-5-yl.

In the present context, common rules for peptide nomenclature based on the three letter amino acid code apply, unless exceptions are specifically indicated. Briefly, the central portion of the amino acid structure is represented by the three letter code (e.g. Ala, Lys) and L-configuration is assumed, unless D-configuration is specifically indicated by “D-” followed by the three letter code (e.g. D-Ala, D-Lys). A substituent at the amino group replaces one hydrogen atom and its name is placed before the three letter code, whereas a C-terminal substituent replaces the carboxylic hydroxy group and its name appears after the three letter code. For example, “acetyl-Gly-Gly-NH₂” represents CH₃—C(═O)—NH—CH₂—C(═O)—NH—CH₂—C(═O)—NH₂. Unless indicated otherwise, amino acids with additional amino or carboxy groups in the side chains (such as Lys, Orn, Dap, Glu, Asp and others) are connected to their neighboring groups by amide bonds formed at the N-2 (α-nitrogen) atom and the C-1 (C═O) carbon atom.

When two amino acids are said to be bridged, it is intended to indicate that functional groups in the side chains of the two respective amino acids have reacted to form a covalent bond.

In the present context, the term “agonist” is intended to indicate a substance (ligand) that activates the receptor type in question.

In the present context, the term “antagonist” is intended to indicate a substance (ligand) that blocks, neutralizes or counteracts the effect of an agonist.

More specifically, receptor ligands may be classified as follows:

Receptor agonists, which activate the receptor; partial agonists also activate the receptor, but with lower efficacy than full agonists. A partial agonist will behave as a receptor partial antagonist, partially inhibiting the effect of a full agonist.

Receptor neutral antagonists, which block the action of an agonist, but do not affect the receptor-constitutive activity.

Receptor inverse agonists, which block the action of an agonist and at the same time attenuate the receptor-constitutive activity. A full inverse agonist will attenuate the receptor-constitutive activity completely; a partial inverse agonist will attenuate the receptor-constitutive activity to a lesser extent.

As used herein the term “antagonist” includes neutral antagonists and partial antagonists, as well as inverse agonists. The term “agonist” includes full agonists as well as partial agonists.

In the present context, the term “pharmaceutically acceptable salt” is intended to indicate a salt which is not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric and nitric acids, and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene-salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. (1977) 66, 2, which is incorporated herein by reference. Examples of relevant metal salts include lithium, sodium, potassium and magnesium salts, and the like. Examples of alkylated ammonium salts include methylammonium, dimethylammonium, trimethylammonium, ethylammonium, hydroxyethylammonium, diethylammonium, butylammonium and tetramethylammonium salts, and the like.

As use herein, the term “therapeutically effective amount” of a compound refers to an amount sufficient to cure, alleviate or partially arrest the clinical manifestations of a given disease and/or its complications. An amount adequate to accomplish this is defined as a “therapeutically effective amount”. Effective amounts for each purpose will depend on the severity of the disease or injury, as well as on the weight and general state of the subject. It will be understood that determination of an appropriate dosage may be achieved using routine experimentation, by constructing a matrix of values and testing different points in the matrix, all of which is within the level of ordinary skill of a trained physician or veterinarian.

The terms “treatment”, “treating” and other variants thereof as used herein refer to the management and care of a patient for the purpose of combating a condition, such as a disease or a disorder. The terms are intended to include the full spectrum of treatments for a given condition from which the patient is suffering, such as administration of the active compound(s) in question to alleviate symptoms or complications thereof, to delay the progression of the disease, disorder or condition, to cure or eliminate the disease, disorder or condition, and/or to prevent the condition, in that prevention is to be understood as the management and care of a patient for the purpose of combating the disease, condition, or disorder, and includes the administration of the active compound(s) in question to prevent the onset of symptoms or complications. The patient to be treated is preferably a mammal, in particular a human being, but treatment of other animals, such as dogs, cats, cows, horses, sheep, goats or pigs, is within the scope of the invention.

As used herein, the term “solvate” refers to a complex of defined stoichiometry formed between a solute (in casu, a compound according to the present invention) and a solvent. Solvents may include, by way of example, water, ethanol, or acetic acid.

The amino acid abbreviations used in the present context have the following meanings:

| | |
|---|---|
| Ala | Alanine |
| β-Ala | 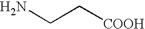 |
| Asn | Asparagine |
| Asn(alkyl) | 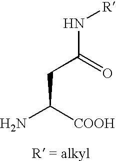 |
| Asn(aryl) | 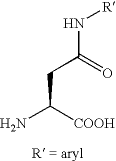 |
| Asp | aspartic acid |
| β-Asp | 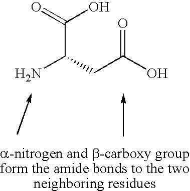 |
| Arg | Arginine |
| Cha | 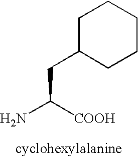 |
| Cgl | 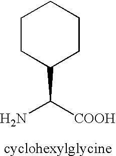 |
| Cit | Citrulline |
| Cys | Cysteine |
| Dab | (S)-2,4-diaminobutyric acid |
| Dap | (S)-2,3-diaminopropionic acid |
| D-β-Asp | 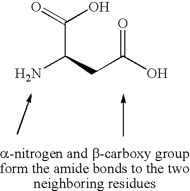 |
| D-γ-Glu | 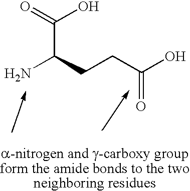 |
| D-Phe | 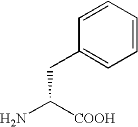 |
| Gln | Glutamine |
| Gln(alkyl) | 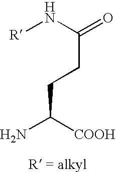 |
| Gln(aryl) | 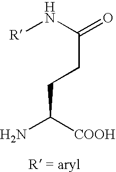 |
| Glu | glutamic acid |
| γ-Glu | 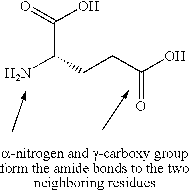 |
| Gly | Glycine |
| His | Histidine |
| homoArg | 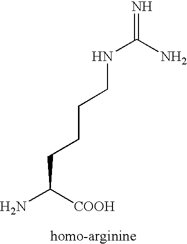 |
| homoCys | 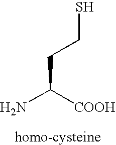 |
| homoSer | 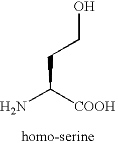 |
| Hyp | 4-hydroxyproline |
| Ile | Isoleucine |
| Leu | Leucine |
| Lys | Lysine |
| Met | Methionine |
| Met(O) | 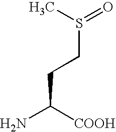 |
| Met(O₂) | 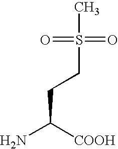 |
| 2-Nal | 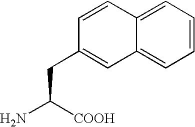 |
| Nle | 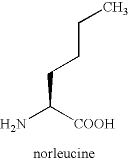 |
| Orn | Ornithine |
| Phe | Phenylalanine |
| Pro | Proline |
| 2-PyAla | 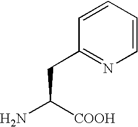 |
| 3-PyAla | 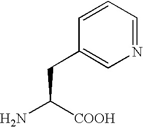 |
| 4-PyAla | 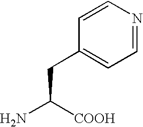 |
| Ser | Serine |
| tBuGly | 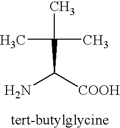 |
| Thr | Threonine |
| (4-thiazolyl)Ala | 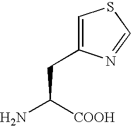 |
| Tic | 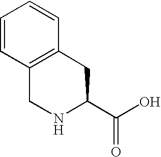 |
| Tyr | Tyrosine |
| Trp | Tryptophan |
| Val | Valine |

Amino acid abbreviations beginning with D- followed by a three letter code, such as D-Ser, D-His and so on, refer to the D-enantiomer of the corresponding amino acid, for example D-serine, D-histidine and so on.

### DESCRIPTION OF THE INVENTION

In certain embodiments of compounds for use in the context of the present invention, the moiety T-A in formula I represents 10-(tetrazol-5-yl)decyl, 11-(tetrazol-5-yl)undecyl, 12-(tetrazol-5-yl)dodecyl, 13-(tetrazol-5-yl)tridecyl, 14-(tetrazol-5-yl)tetradecyl, 15-(tetrazol-5-yl)pentadecyl, 16-(tetrazol-5-yl)hexadecyl, 17-(tetrazol-5-yl)heptadecyl; 18-(tetrazol-5-yl)octadecyl or 19-(tetrazol-5-yl)nonadecyl.

In certain other embodiments of the compounds, S¹ in formula II is absent.

In further embodiments of the compounds, S¹ in formula II represents a structure according to formula IIIa.

In additional embodiments of the compounds, S¹ in formula II represents a structure according to formula IIIb.

In still further embodiments of the compounds, S¹ in formula II represents a structure according to formula IIIc.

In certain other embodiments of the compounds, Z¹ in formula II is absent, or Z¹ in formula II represents Gly.

In further embodiments of the compounds, Z² in formula II represents Ser, Thr, Gln, Gly or His, such as Ser or Thr.

In additional embodiments of the compounds, Z³ in formula II represents Gln, D-Gln, Asn, D-Asn, Ser or D-Ser.

In further embodiments of the compounds, S² in formula IVa, IVb or IVc represents a structure according to formula IIIa or formula IIIb.

In some embodiments of the compounds, the moiety R¹-R² (i.e. R¹ and R² taken together) in formula II or in formula IVa, IVb or IVc represents 10-(tetrazol-5-yl)decyl, 11-(tetrazol-5-yl)undecyl, 12-(tetrazol-5-yl)dodecyl, 13-(tetrazol-5-yl)tridecyl, 14-(tetrazol-5-yl)tetradecyl, 15-(tetrazol-5-yl)pentadecyl, 16-(tetrazol-5-yl)hexadecyl, 17-(tetrazol-5-yl)heptadecyl, 18-(tetrazol-5-yl)octadecyl or 19-(tetrazol-5-yl)nonadecyl, such as 13-(tetrazol-5-yl)tridecyl, 14-(tetrazol-5-yl)tetradecyl, 15-(tetrazol-5-yl)pentadecyl, 16-(tetrazol-5-yl)hexadecyl or 17-(tetrazol-5-yl )heptadecyl, e.g. 15-(tetrazol-5-yl)pentadecyl.

In other embodiments, the moiety R¹-R² (i.e. R¹ and R² taken together) in formula II or in formula IVa, IVb or IVc represents 12-carboxydodecyl, 13-carboxytridecyl, 14-carboxytetradecyl, 15-carboxypentadecyl, 16-carboxyhexadecyl, 17-carboxyheptadecyl, 18-carboxyoctadecyl or 19-carboxynonadecyl, such as 14-carboxytetradecyl or 16-carboxytetradecyl.

In certain embodiments of the compounds, R³ in formula II or in formula IVa, IVb or IVc is absent. In other embodiments, R³ in formula II or in formula IVa, IVb or IVc represents —NH—S(═O)₂—(CH₂)₃₋₅—C(═O)—, Glu, D-Glu, γ-Glu, D-γ-Glu, Asp, D-Asp, β-Asp, D-β-Asp or Gly-γ-Glu. In some embodiments, R³ in formula II or in formula IVa, IVb or IVc represents —NH—S(═O)₂—(CH₂)₃—C(═O)—. In other embodiments, R³ represents D-Glu, γ-Glu, β-Asp or Gly-γ-Glu.

In additional embodiments of the compounds, Z⁴ in formula II or in formula IVa represents Ser, homoSer, Gln, Asn, Tyr, His, Arg, homoArg, Lys, Orn, Dab or Dap, such as Ser, His, Arg or Dap.

In further embodiments of the compounds, Z⁵ in formula II or in formula IVa or IVb represents Ser, homoSer, Thr, Pro, His, Hyp, Lys, Orn, Dab or Dap, such as Ser, His or Dap.

In certain embodiments of the compounds, Z⁶ in formula II or in formula IVa, IVb or IVc represents Ala, Val, Leu, Ile, Met or Nle, such as Nle.

In additional embodiments of the compounds X² in formula II or in formula IVa, IVb or IVc represents Ser, Hyp, Cit, Dap, Asn, Gln or (4-thiazolyl)Ala, such as Hyp, Dap, Cit or Gln, e.g. Hyp.

In a group of embodiments of the compounds, X¹ is Glu, X³ is D-Phe, X⁴ is Trp and X⁵ is Lys. In another group of embodiments, X¹ is Asp, X³ is D-Phe, X⁴ is Trp and X⁵ is Lys.

In a particular group of embodiments of the compounds, R⁴ in formula II or in formula IVa, IVb or IVc is NH₂. In another group of embodiments, R⁴ is OH.

Specific examples of compounds of the type in question are the following, each of which individually constitutes an embodiment of a compound for use in the context of the invention:

### 16-(Tetrazol-5-yl)hexadecanoyl-Gly-Thr-Gln-His-Ser-Nle-c[Glu-Hyp-D-Phe-Arg-Trp-Lys]-NH₂

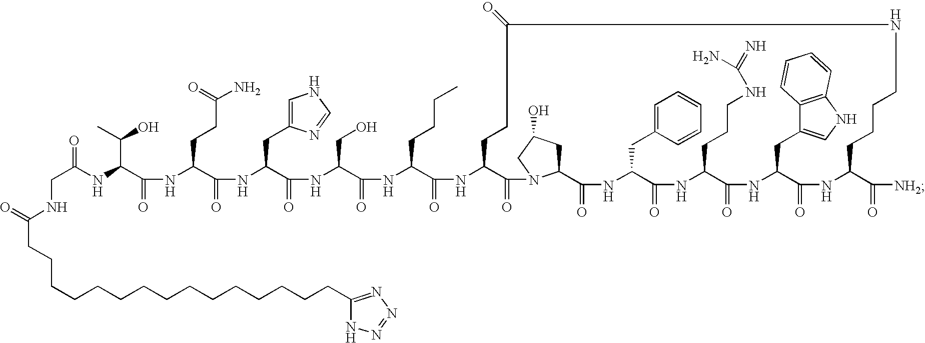

### 16-(Tetrazol-5-yl)hexadecanoyl-Gly-Thr-Gln-Dap-Ser-Nle-c[Glu-Hyp-D-Phe-Arg-Trp-Lys]-NH₂

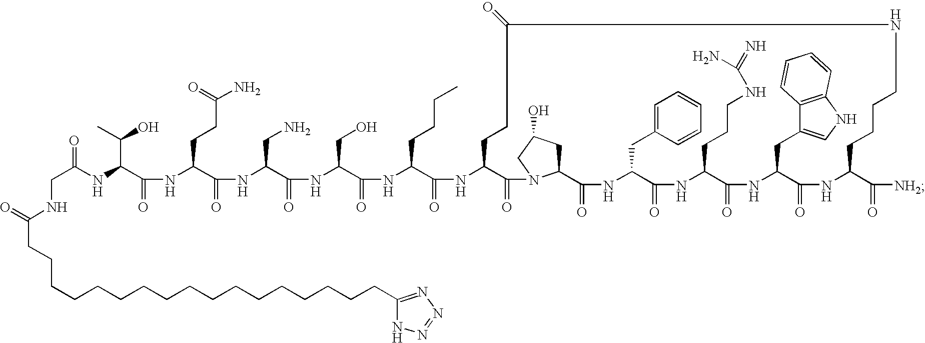

### {2-[2-(16-(Tetrazol-5-yl)hexadecanoylamino)ethoxy]ethoxy}acetyl-Gly-Thr-Gln-Dap-Ser-Nle-c[Glu-Hyp-D-Phe-Arg-Trp-Lys]-NH₂

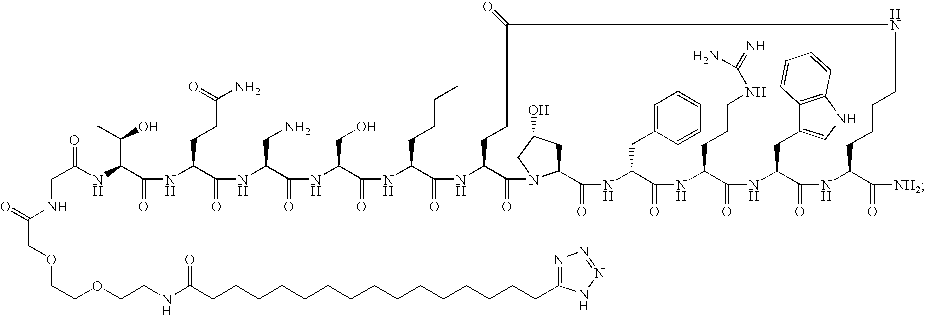

### {2-[2-(16-(Tetrazol-5-yl)hexadecanoylamino)ethoxy]ethoxy}acetyl-Gly-Ser-Gln-His-Ser-Nle-c[Glu-Dap-D-Phe-Arg-Trp-Lys]-NH₂

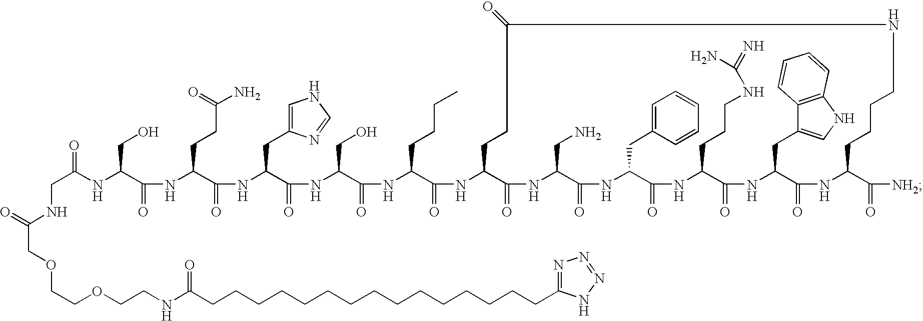

### {2-[2-(16-(Tetrazol-5-yl)hexadecanoylamino)ethoxy]ethoxy}acetyl-Gly-Ser-Gln-His-Dap-Nle-c[Glu-Hyp-D-Phe-Arg-Trp-Lys]-NH₂

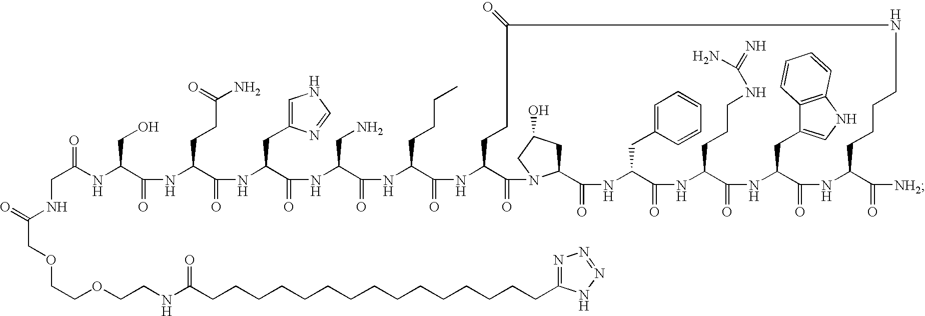

### 4-(16-(Tetrazol-5-yl)hexadecanoylsulfamoyl)butanoyl-Gly-Ser-D-Gln-His-Dap-Nle-c[Glu-Hyp-D-Phe-Arg-Trp-Lys]-NH₂

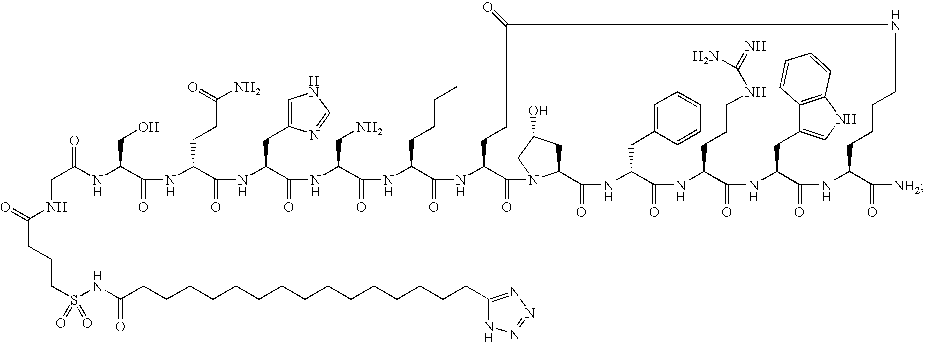

### {2-[2-(2-{2-[2-(16-(Tetrazol-5-yl)hexadecanoylamino)ethoxy]ethoxy}acetylamino)ethoxy]-ethoxy}acetyl-Nle-c[Glu-Dap-D-Phe-Arg-Trp-Lys]-NH₂

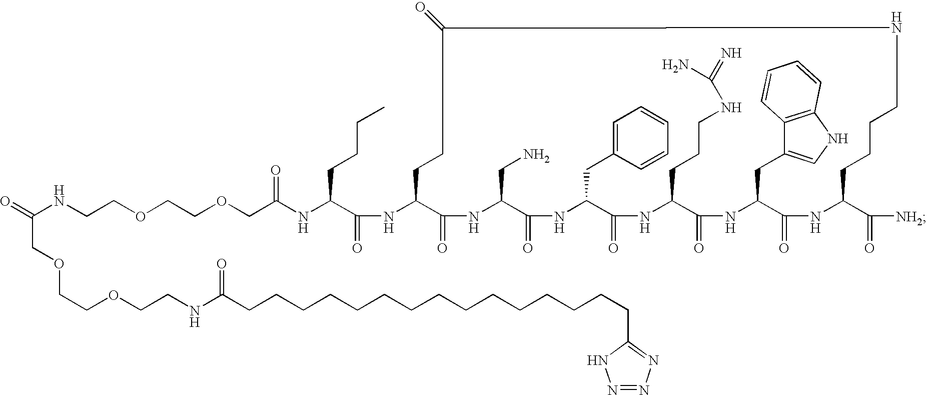

### (2-{2-[4-(16-(Tetrazol-5-yl)hexadecanoylsulfamoyl)butanoylamino]ethoxy}ethoxy)acetyl-His-Nle-c[Glu-Dap-D-Phe-Arg-Trp-Lys]-NH₂

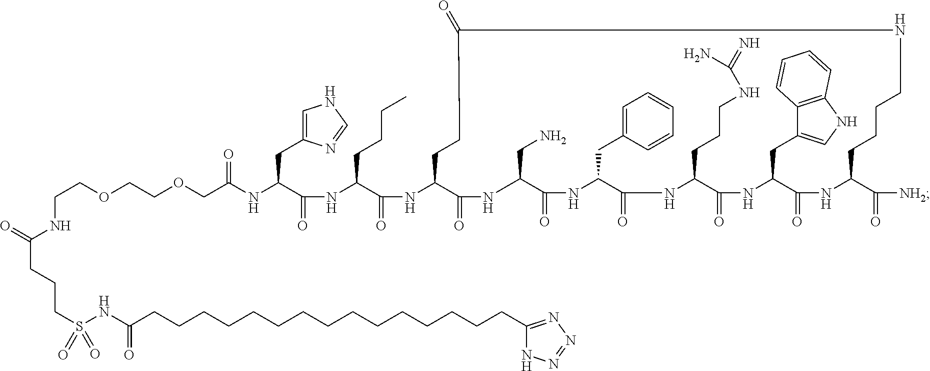

### {2-[2-(2-{2-[2-(15-Carboxypentadecanoylamino)ethoxy]ethoxy}acetylamino)ethoxy]ethoxy}-acetyl-Pro-Nle-c[Glu-Hyp-D-Phe-Arg-Trp-Lys]-NH₂

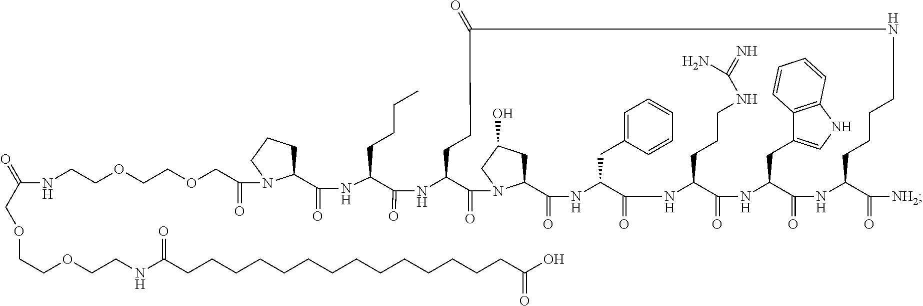

### {2-[2-(2-{2-[2-(15-Carboxypentadecanoylamino)ethoxy]ethoxy}acetylamino)ethoxy]ethoxy}-acetyl-Nle-c[Glu-Hyp-D-Phe-Arg-Trp-Lys]-NH₂

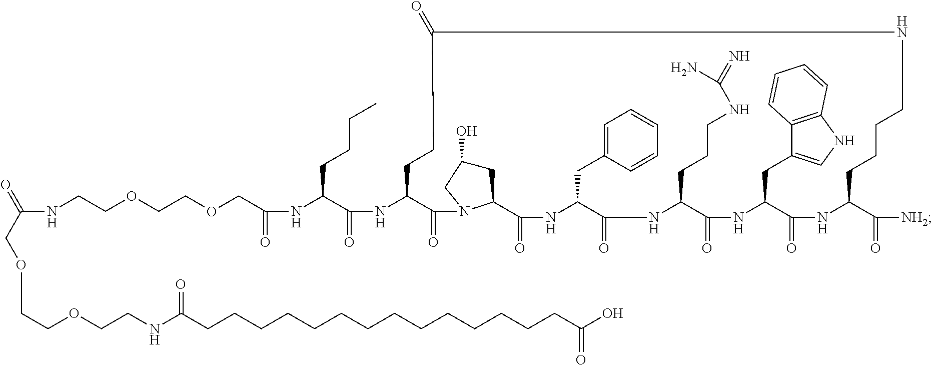

the compound:

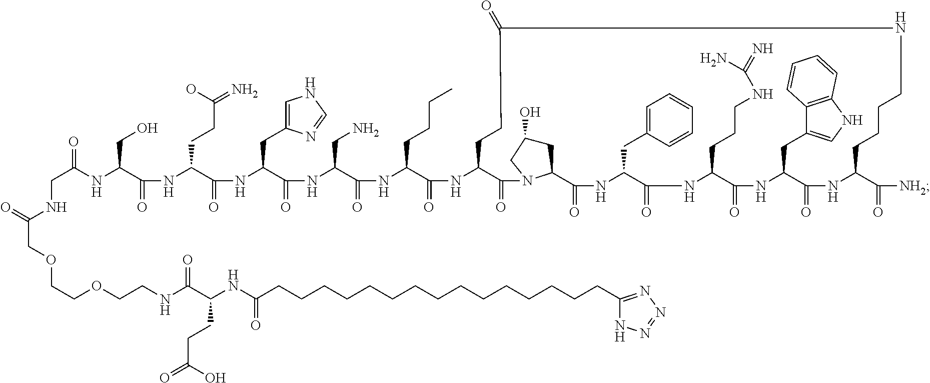

the compound:

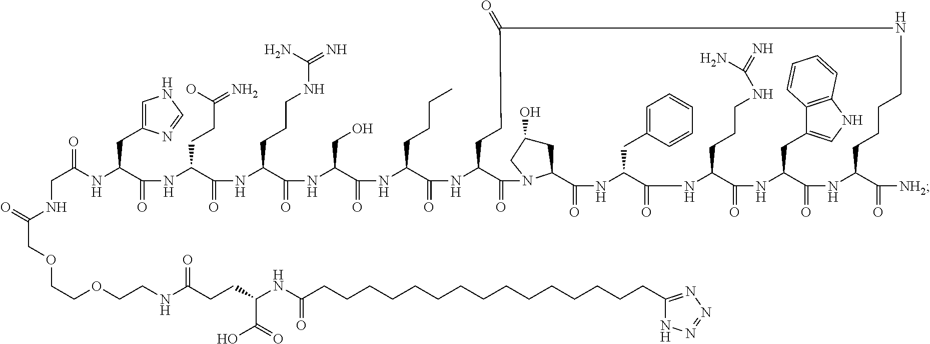

the compound:

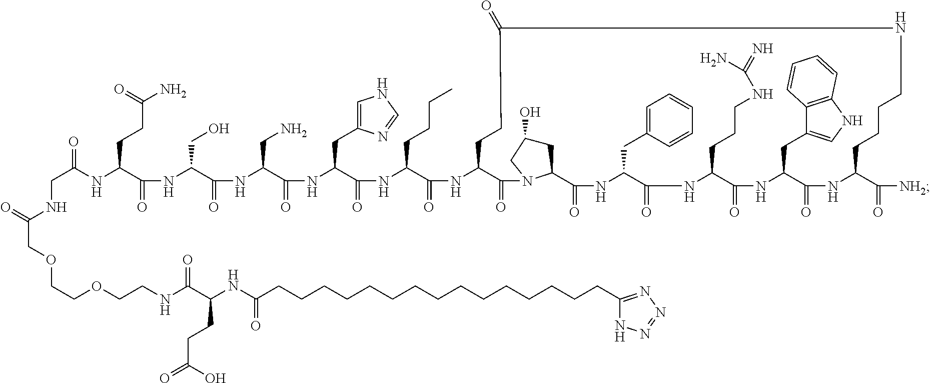

the compound:

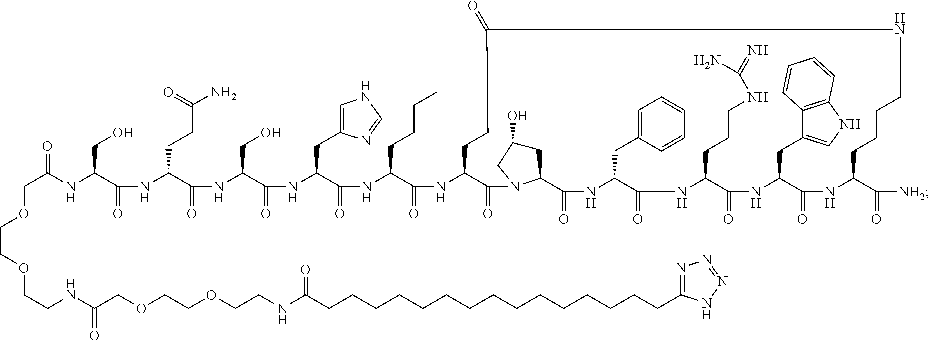

the compound:

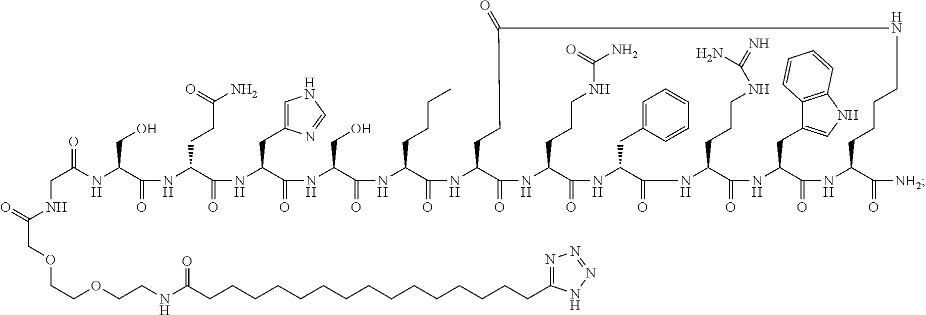

the compound:

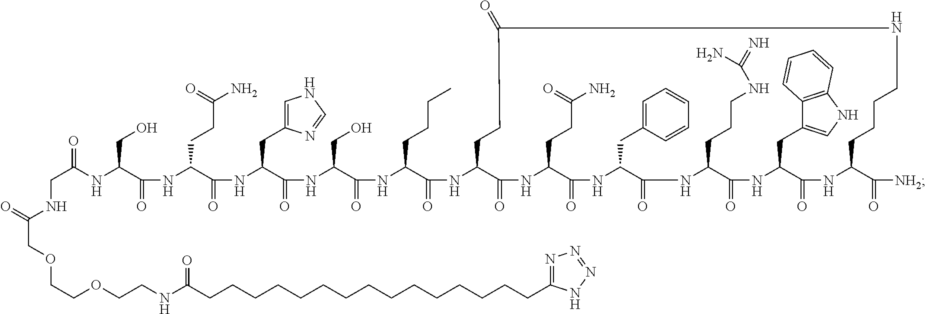

### (2-{2-[2-(2-{2-[(R)-4-Carboxy-2-(16-(1H-tetrazol-5-yl)hexadecanoylamino)butanoylamino]-ethoxy}ethoxy)acetylamino]ethoxy}ethoxy)acetyl-Ser-Ser-Nle-c[Glu-Hyp-D-Phe-Arg-Trp-Lys]-NH₂

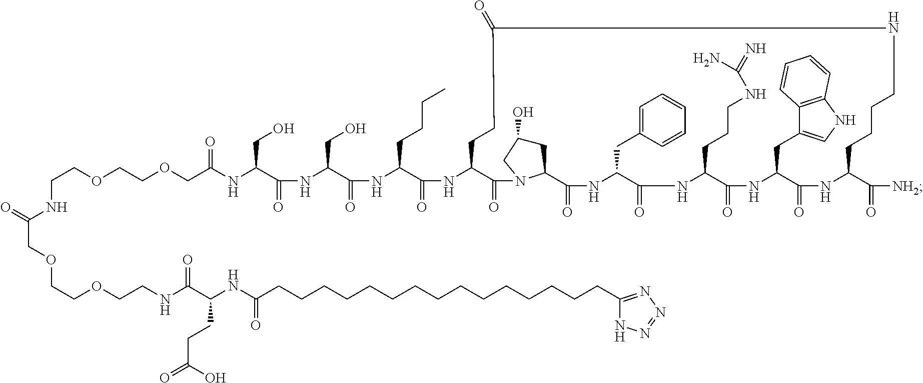

the compound:

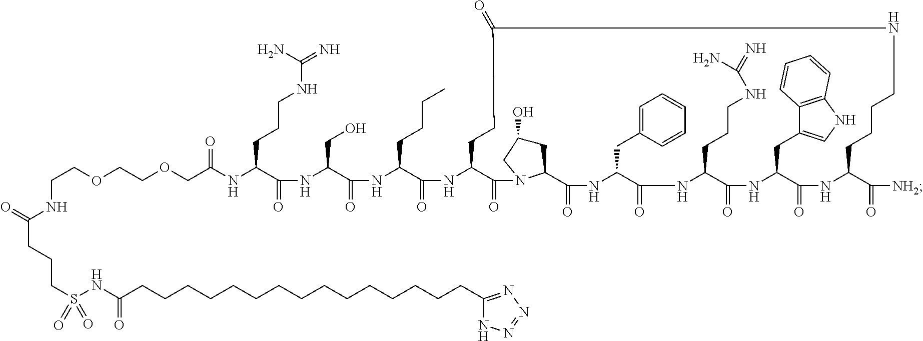

the compound:

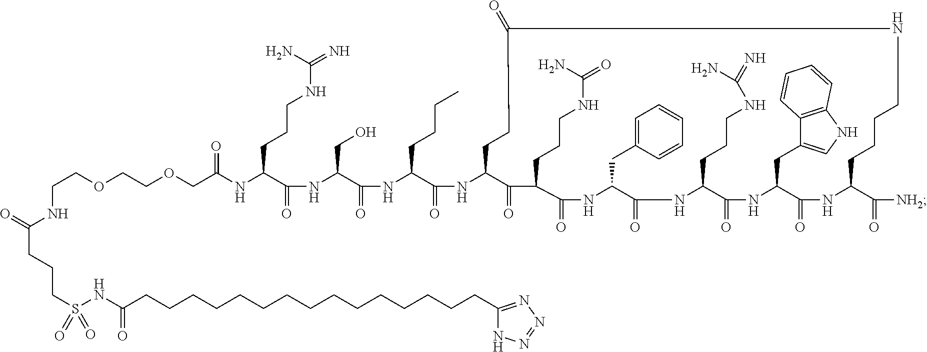

### {2-[2-(15-(Carboxy)pentadecanoylamino)ethoxy]ethoxy}acetyl-Gly-Ser-Gln-His-Dap-Nle-c[Glu-Hyp-D-Phe-Arg-Trp-Lys]-NH₂

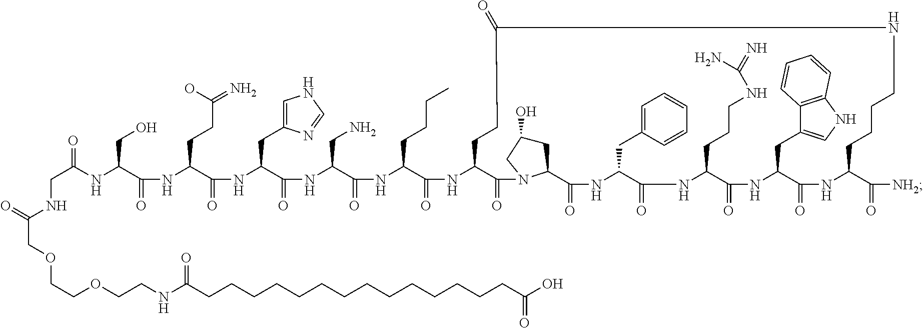

the compound:

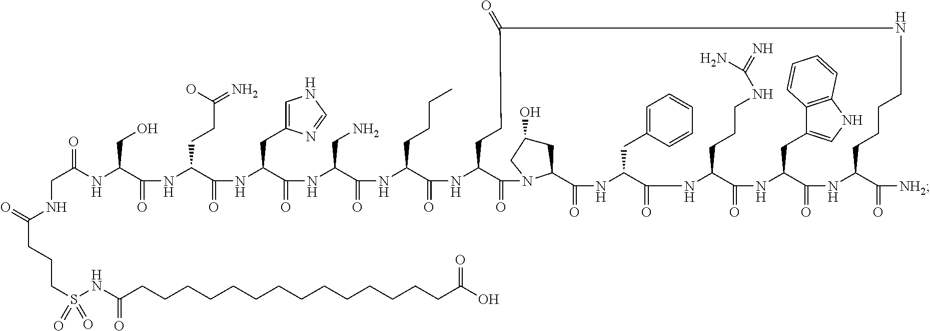

the compound:

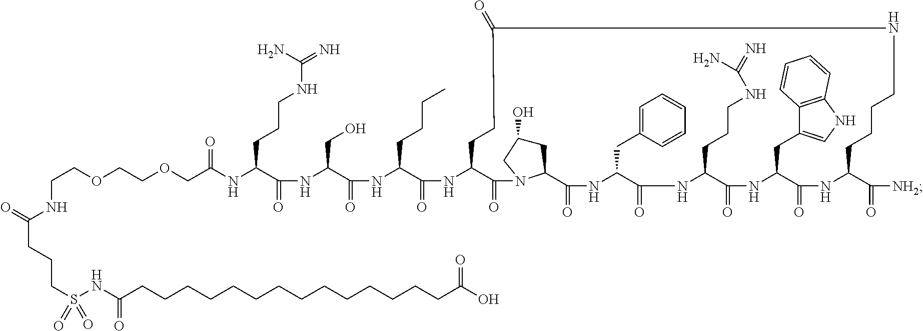

the compound:

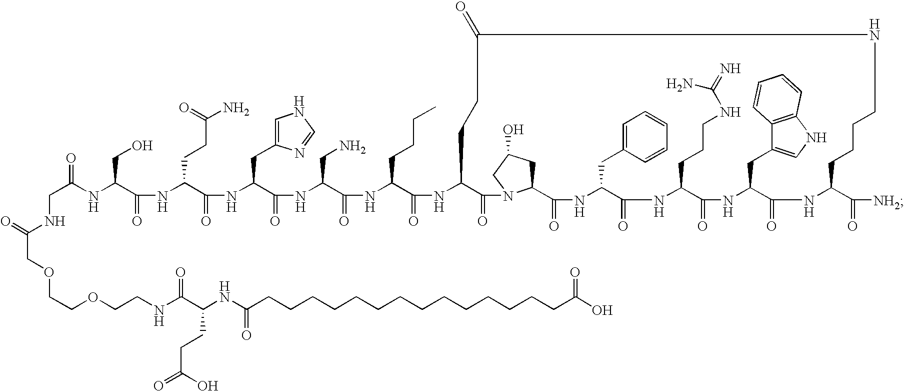

### 15-Carboxypentadecanoyl-Gly-Ser-Ser-Tyr-Thr-Nle-c[Glu-Hyp-D-Phe-Arg-Trp-Lys]-NH₂

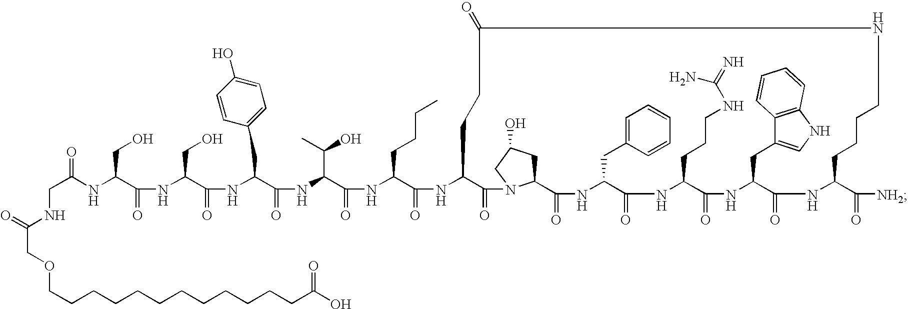

### {2-[2-(15-Carboxypentadecanoylamino)ethoxy]ethoxy}acetyl-Ser-Tyr-Hyp-Nle-c[Glu-Hyp-D-Phe-Arg-Trp-Lys]-NH₂

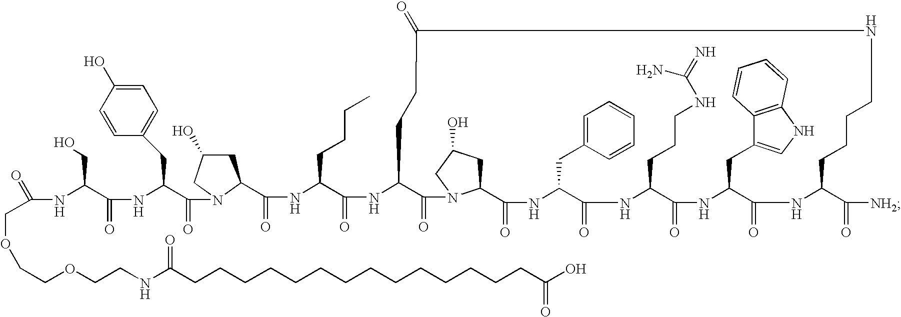

### {2-[2-(15-Carboxypentadecanoylamino)ethoxy]ethoxy}acetyl-Asn-Asn-Pro-Nle-c[Glu-Hyp-D-Phe-Arg-Trp-Lys]-NH₂

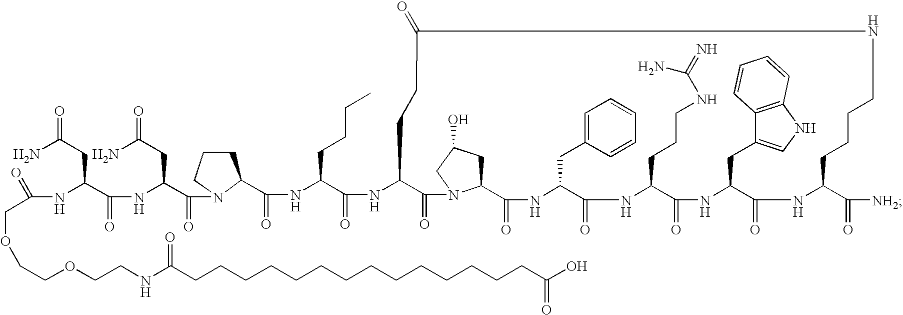

### (2-{2-[(R)-4-Carboxy-2-(16-(tetrazol-5-yl)hexadecanoylamino)butanoylamino]ethoxy}-ethoxy)acetyl-Gly-Ser-Gln-His-Dap-Nle-c[Glu-Hyp-D-Phe-Arg-Trp-Lys]-NH₂

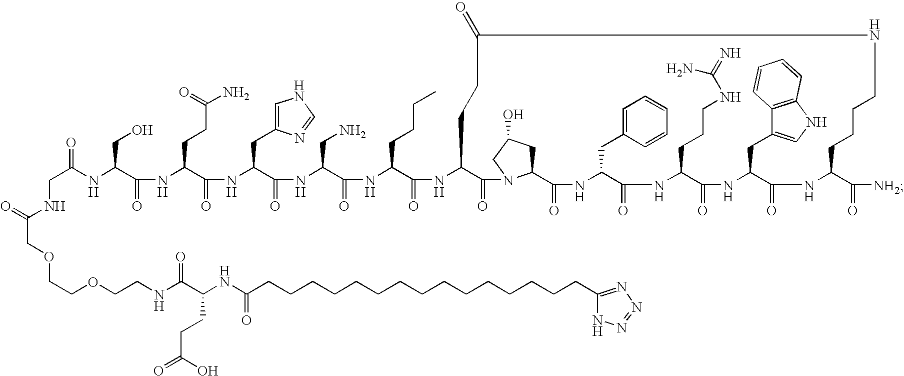

### {2-[2-(2-{2-[2-(16-(Tetrazol-5-yl)hexadecanoylamino)ethoxy]ethoxy}acetylamino)ethoxy]-ethoxy}acetyl-Ser-Gln-His-Dap-Nle-c[Glu-Hyp-D-Phe-Arg-Trp-Lys]-NH₂

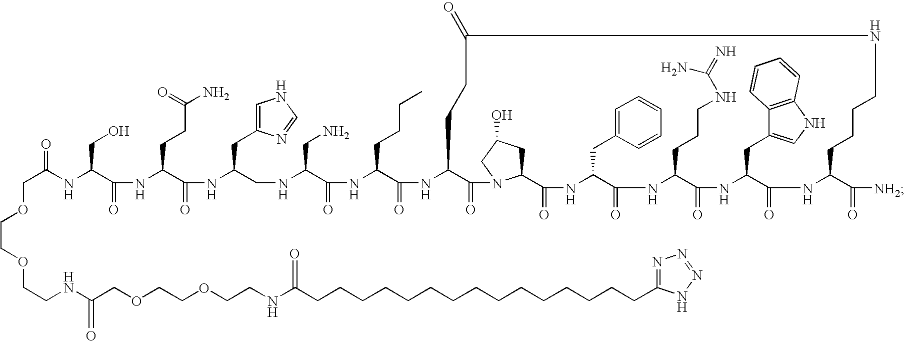

### (2-{2-[4-(16-(Tetrazol-5-yl)hexadecanoylsulfamoyl)butanoylamino]ethoxy}ethoxy)acetyl-Arg-Dap-Nle-c[Glu-Hyp-D-Phe-Arg-Trp-Lys]-NH₂

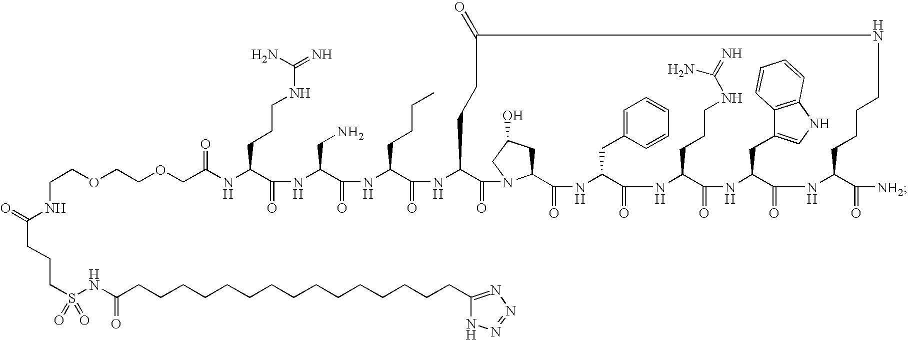

### {2-[2-(16-(Tetrazol-5-yl)hexadecanoylamino)ethoxy]ethoxy}acetyl-Gly-Ser-Gln-Dap-Ser-Nle-c[Glu-Hyp-D-Phe-Arq-Trp-Lys]-NH₂

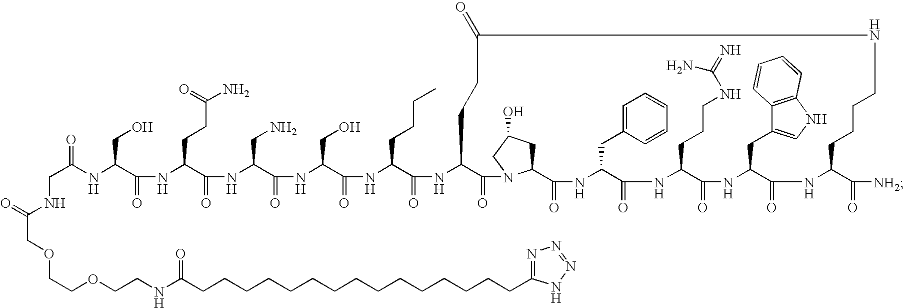

### (2-{2-[4-(16-(Tetrazol-5-yl)hexadecanoylsulfamoyl)butanoylamino]ethoxy}ethoxy)acetyl-His-Dap-Nle-c[Glu-Hyp-D-Phe-Arg-Trp-Lys]-NH₂

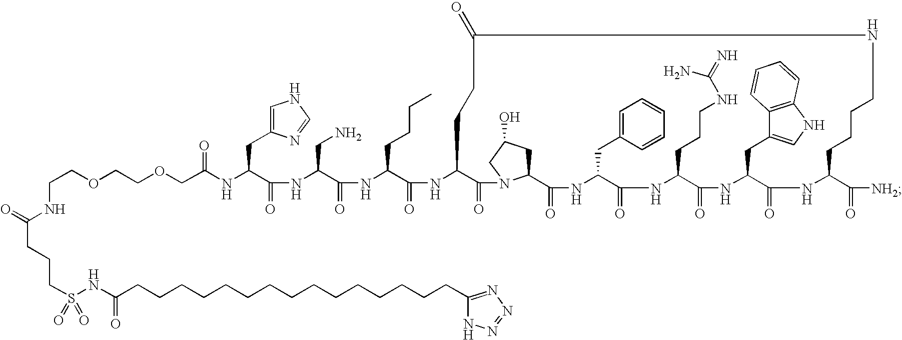

### {2-[2-(2-{2-[2-(16-(Tetrazol-5-yl)hexadecanoylamino)ethoxy]ethoxy}acetylamino)ethoxy]ethoxy}acetyl-Dap-Nle-c[Glu-Hyp-D-Phe-Arg-Trp-Lys]-NH₂

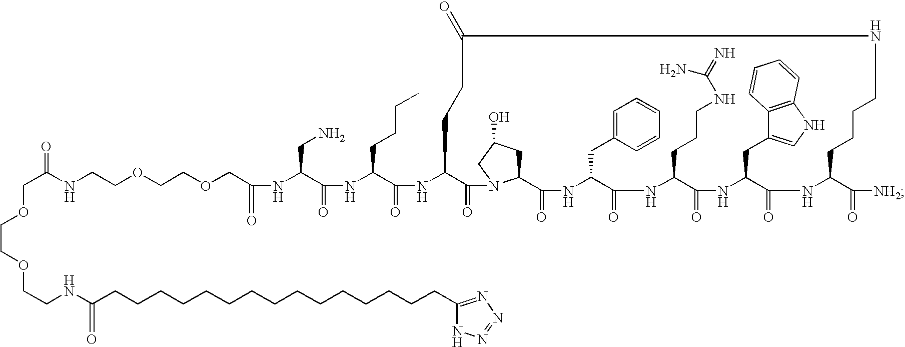

### (2-{2-[4-(16-(Tetrazol-5-yl)hexadecanoylsulfamoyl)butanoylamino]ethoxy}ethoxy)acetyl-Gly-Ser-Gln-His-Dap-Nle-c[Glu-Hyp-D-Phe-Arg-Trp-Lys]-NH₂

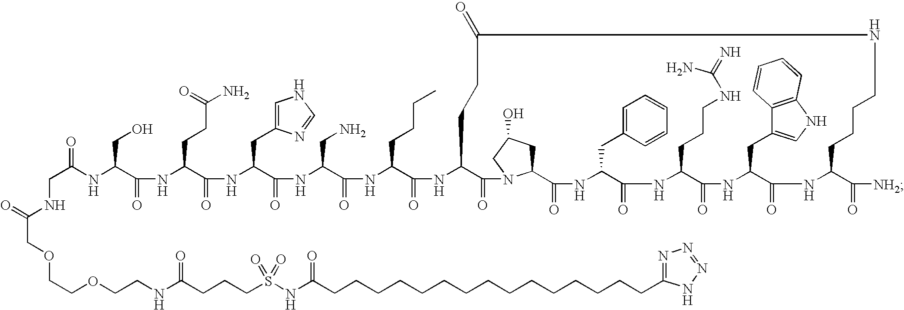

### 4-(16-(Tetrazol-5-yl)hexadecanoylsulfamoyl)butanoyl-Gly-Ser-Gln-His-Dap-Nle-c[Glu-Hyp-D-Phe-Arg-Trp-Lys]-NH₂

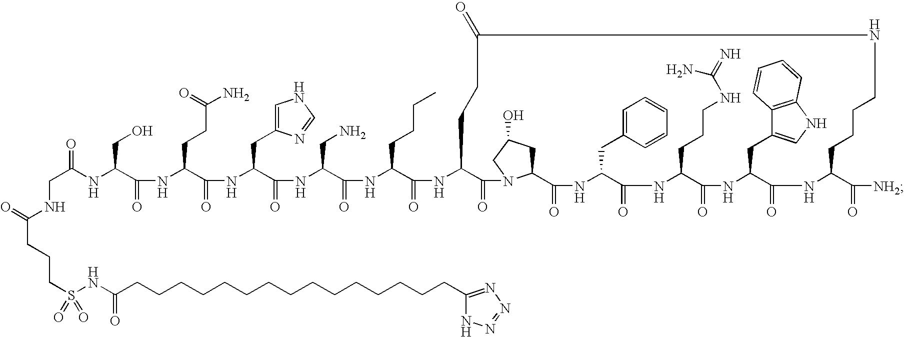

### {2-[2-(16-(Tetrazol-5-yl)hexadecanoylamino)ethoxy]ethoxy}acetyl-Gly-Ser-D-Ser-His-His-Nle-c[Glu-Hyp-D-Phe-Arg-Trp-Lys]-OH

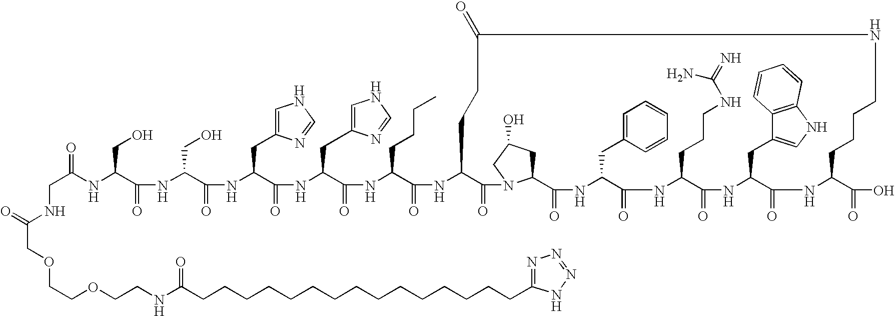

### (2-[2-{(2-[2-{16-(Tetrazol-5-yl)hexadecanoylamino}ethoxy]ethoxy)acetylamino}ethoxy]-ethoxy)acetyl-Gly-Ser-Gln-His-Dap-Nle-c[Glu-Hyp-D-Phe-Arg-Trp-Lys]-NH₂

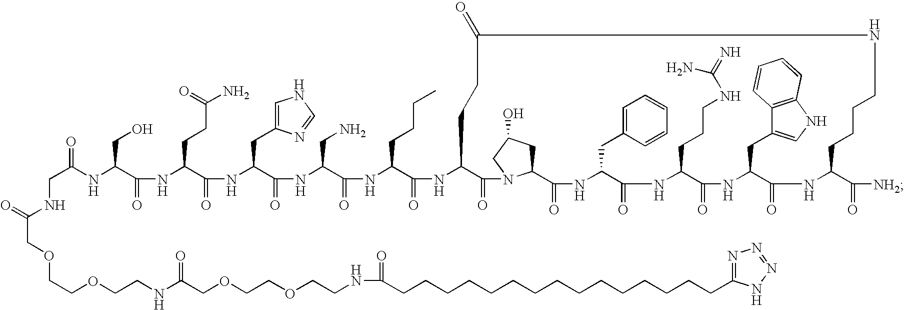

### (2-[2-{(2-[2-{(2-[2-{(2-[2-{16-(Tetrazol-5-yl)hexadecanoylamino}ethoxy]ethoxy)acetylamino}-ethoxy]ethoxy)acetylamino}ethoxy]ethoxy)acetylamino}ethoxy]ethoxy)acetyl-Gly-Ser-Gln-His-Dap-Nle-c[Glu-Hyp-D-Phe-Arg-Trp-Lys]-NH₂

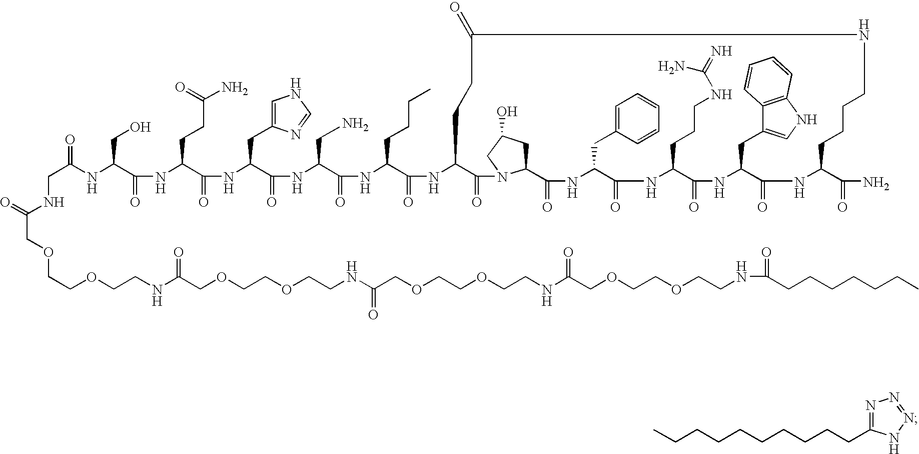

### {2-[2-(16-(Tetrazol-5-yl)hexadecanoylamino)ethoxy]ethoxy}acetyl-Gly-Ser-Gln-His-His-Nle-c[Glu-Hyp-D-Phe-Arg-Trp-Lys]-NH₂

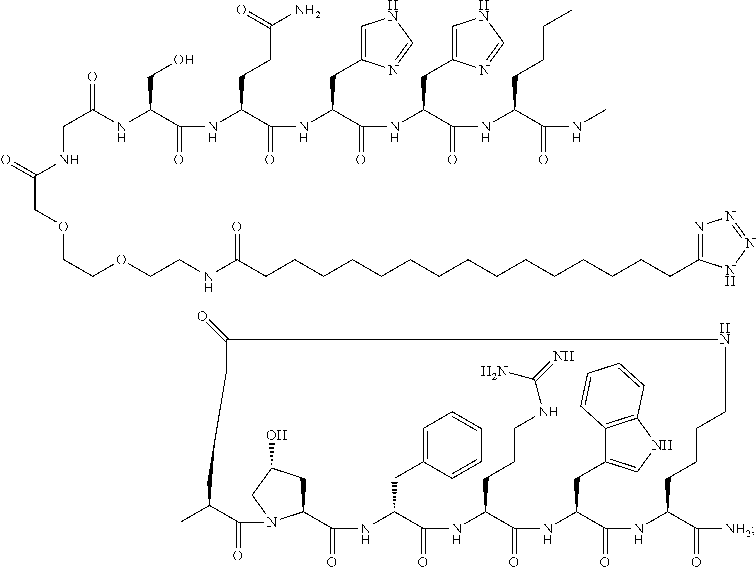

### {2-[2-(16-(Tetrazol-5-yl)hexadecanoylamino)ethoxy]ethoxy}acetyl-Gly-Ser-Gln-His-Ser-Nle-c[Glu-Hyp-D-Phe-Arg-Trp-Lys]-NH₂

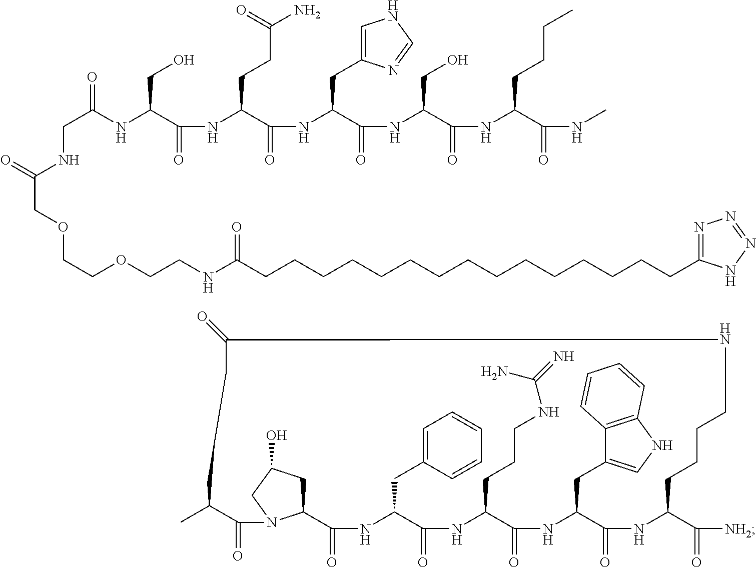

### {2-[2-(16-(Tetrazol-5-yl)hexadecanoylamino)ethoxy]ethoxy}acetyl-Gly-Ser-Gln-His-Nle-c[Glu-Hyp-D-Phe-Arg-Trp-Lys]-NH₂

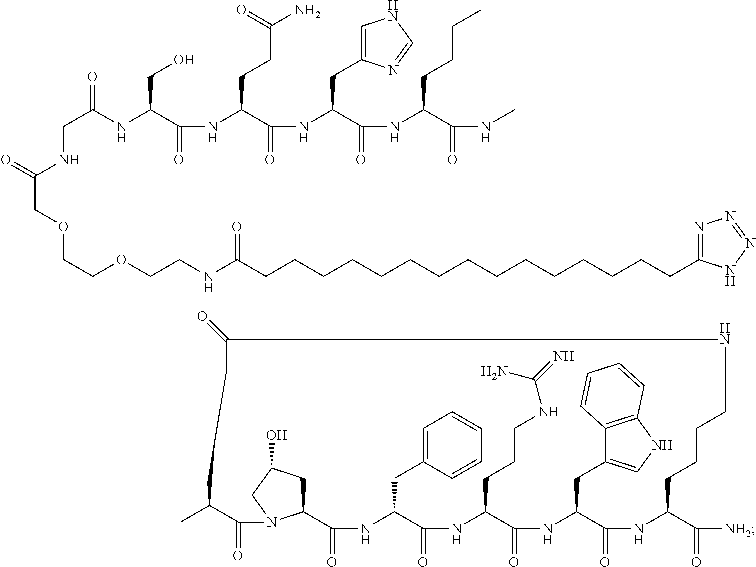

### 4-(15-Carboxypentadecanoylsulfamoyl)butanoyl-Gly-Ser-Gln-His-Dap-Nle-c[Glu-Hyp-D-Phe-Arq-Trp-Lysl-NH₂

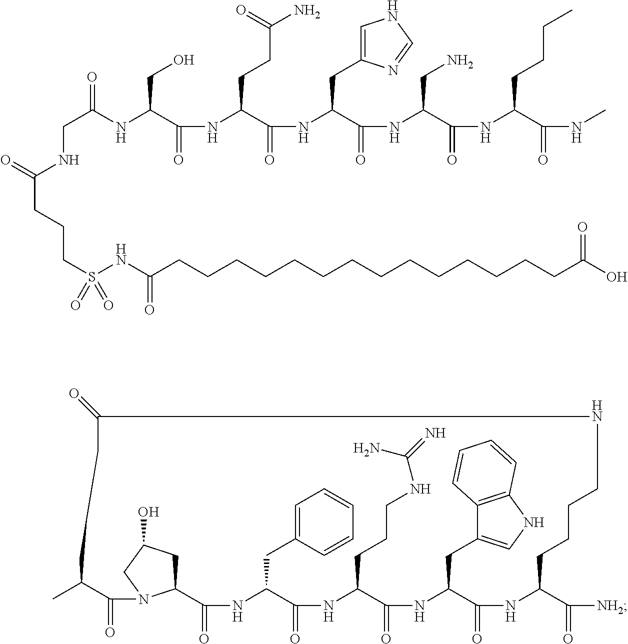

### (2-{2-[(S)-4-Carboxy-4-(17-carboxyheptadecanoylamino)butanoylamino]ethoxy}ethoxy)-acetyl-Gly-Ser-Gln-His-Dap-Nle-c[Glu-Hyp-D-Phe-Arg-Trp-Lys]-NH₂

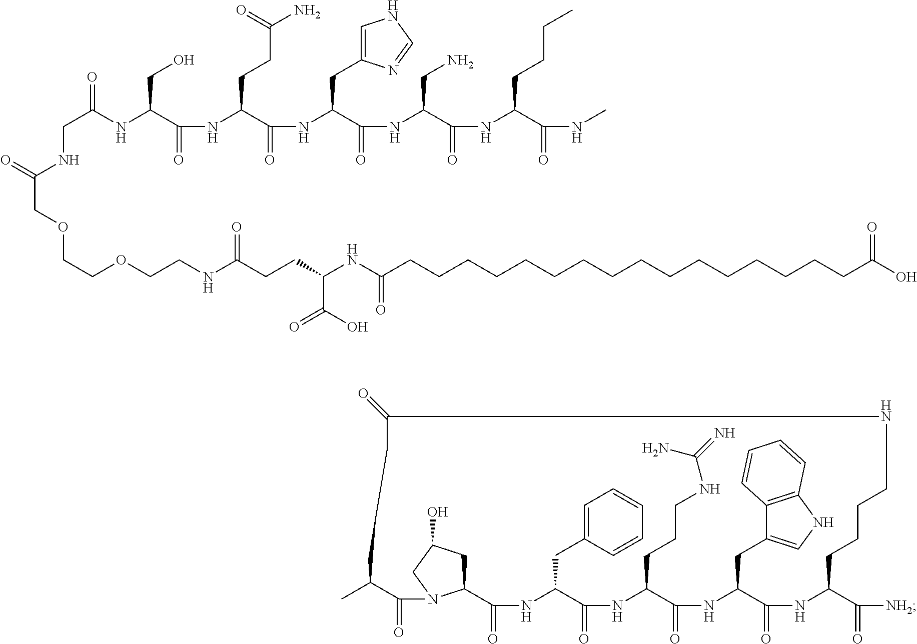

### [2-(2-{(S)-4-Carboxy-4-[2-(17-carboxyheptadecanoylamino)acetylamino]butanoylamino}-ethoxy)ethoxy]acetyl-Gly-Ser-Gln-His-Dap-Nle-c[Glu-Hyp-D-Phe-Arg-Trp-Lys]-NH₂

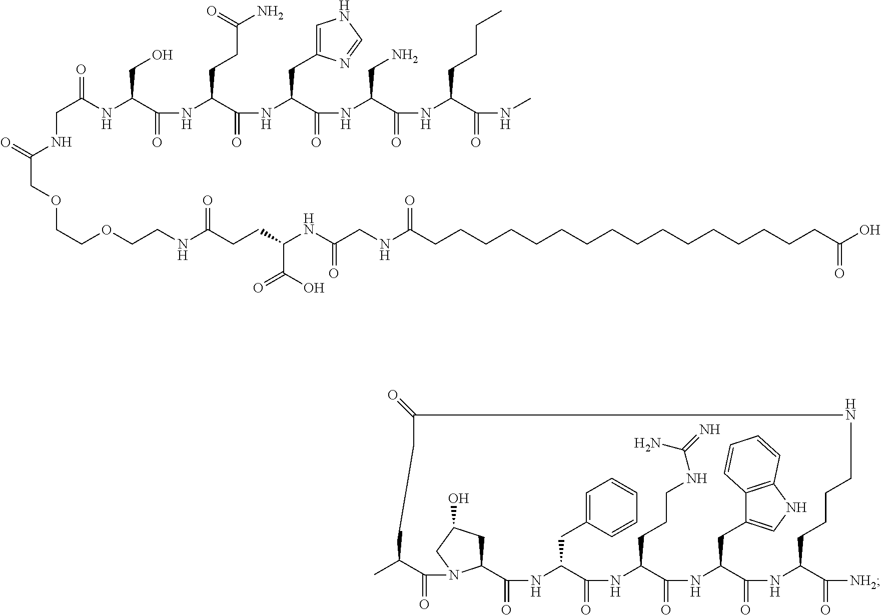

### (2-{2-[(S)-3-Carboxy-3-(17-carboxyheptadecanoylamino)propanoylamino]ethoxy}ethoxy)-acetyl-Gly-Ser-Gln-His-Dap-Nle-c[Glu-Hyp-D-Phe-Arg-Trp-Lys]-NH₂

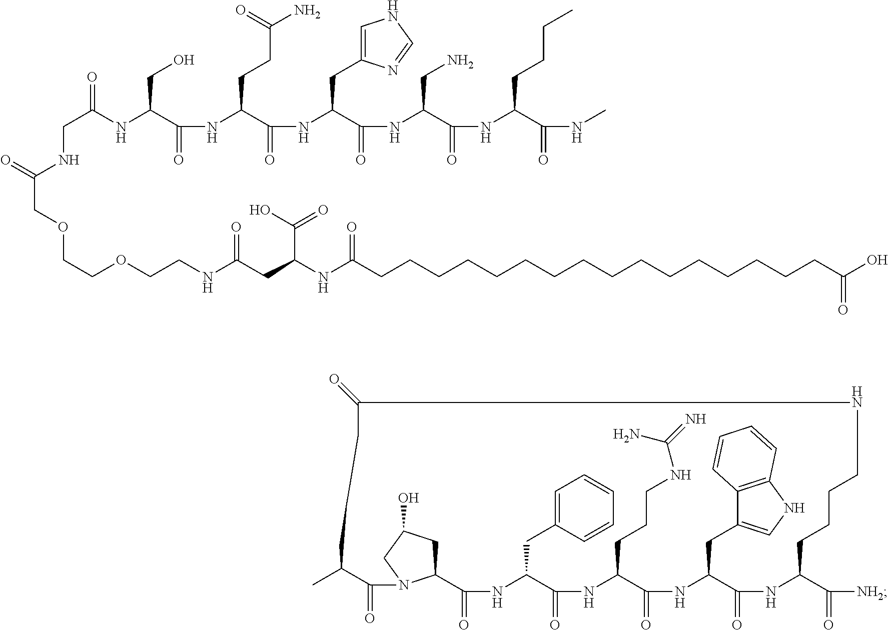

### {2-[2-(16-(Tetrazol-5-yl)hexadecanoylamino)ethoxy]ethoxy}acetyl-Gly-Ser-Gln-His-Thr-Nle-c[Glu-Hyp-D-Phe-Arg-Trp-Lys]-NH₂

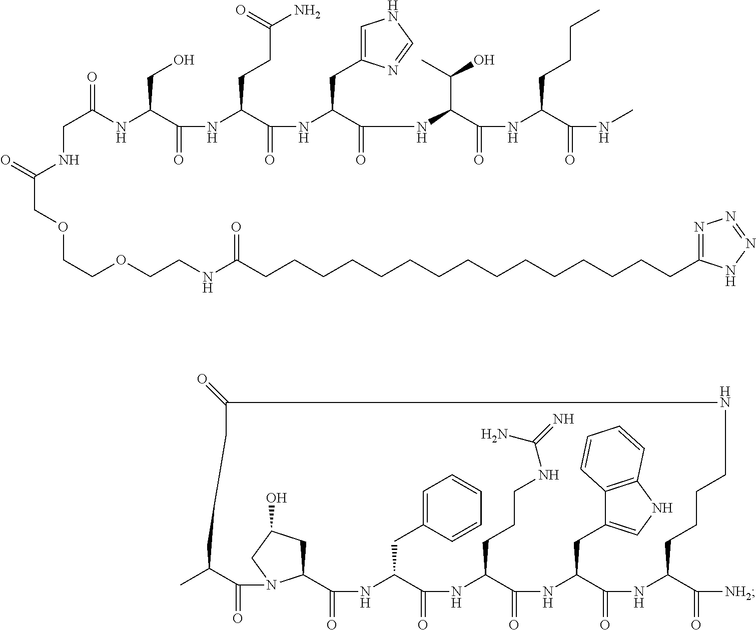

### {2-[2-(16-(Tetrazol-5-yl)hexadecanoylamino)ethoxy]ethoxy}acetyl-Gly-Ser-Gln-His-Dab-Nle-c[Glu-Hyp-D-Phe-Arg-Trp-Lys]-NH₂

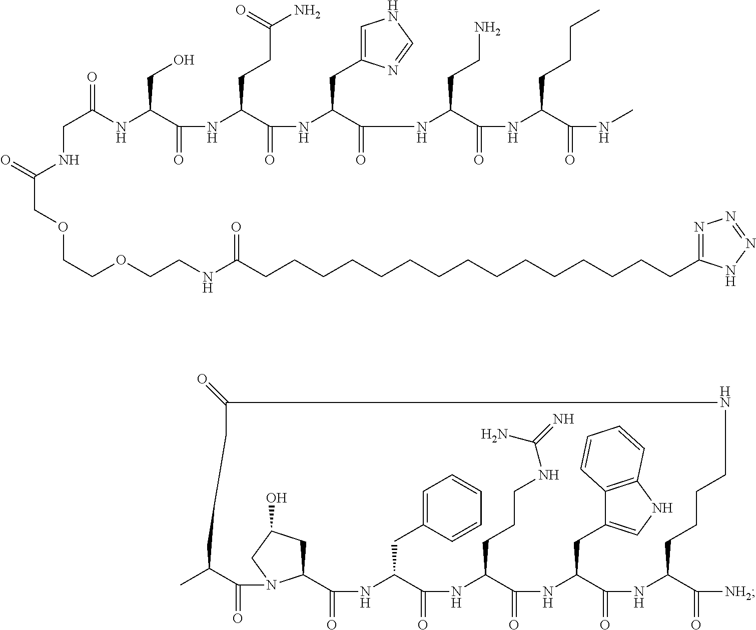

### {2-[2-(16-(Tetrazol-5-yl)hexadecanoylamino)ethoxy]ethoxy}acetyl-Gly-Ser-Gln-His-homoSer-Nle-c[Glu-Hyp-D-Phe-Arg-Trp-Lys]-NH₂

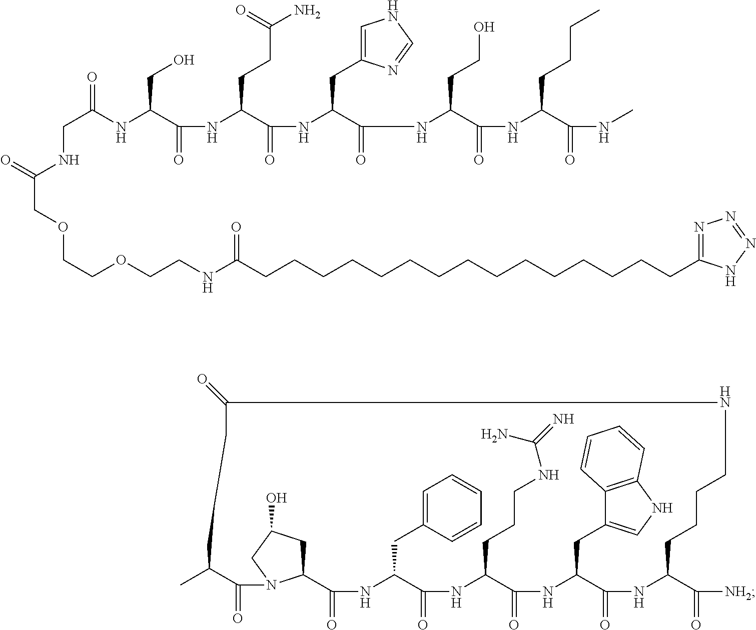

### {2-[2-(16-(Tetrazol-5-yl)hexadecanoylamino)ethoxy]ethoxy}acetyl-Gly-Ser-Gln-His-Orn-Nle-c[Glu-Hyp-D-Phe-Arg-Trp-Lys]-NH₂

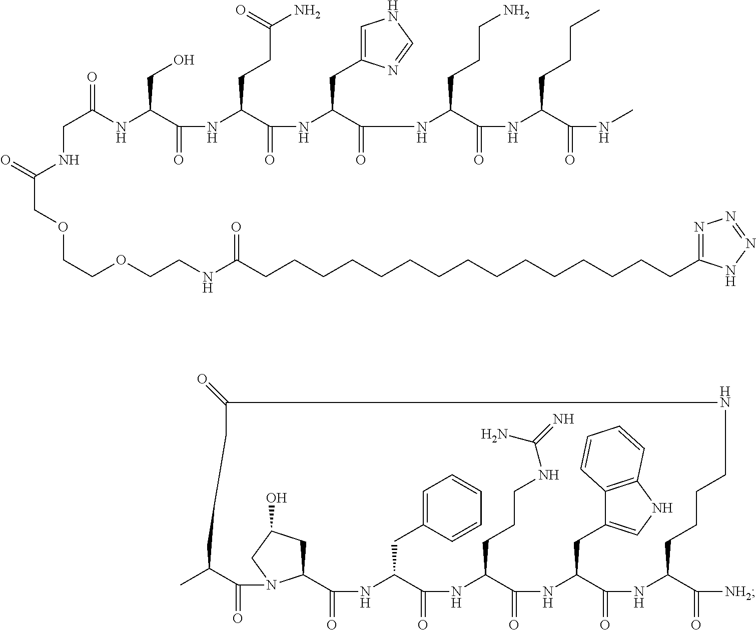

### {2-[2-(16-(Tetrazol-5-yl)hexadecanoylamino)ethoxy]ethoxy}acetyl-Gly-Ser-Gln-His-Lys-Nle-c[Glu-Hyp-D-Phe-Arg-Trp-Lys]-NH₂

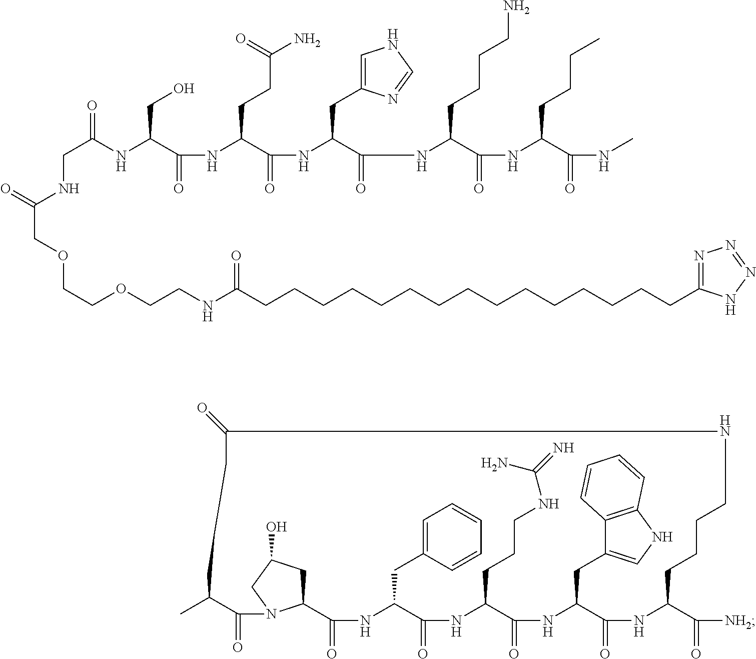

### {2-[2-(16-(Tetrazol-5-yl)hexadecanoylamino)ethoxy]ethoxy}acetyl-Gly-Ser-Gln-His-Arg-Nle-c[Glu-Hyp-D-Phe-Arg-Trp-Lys]-NH₂

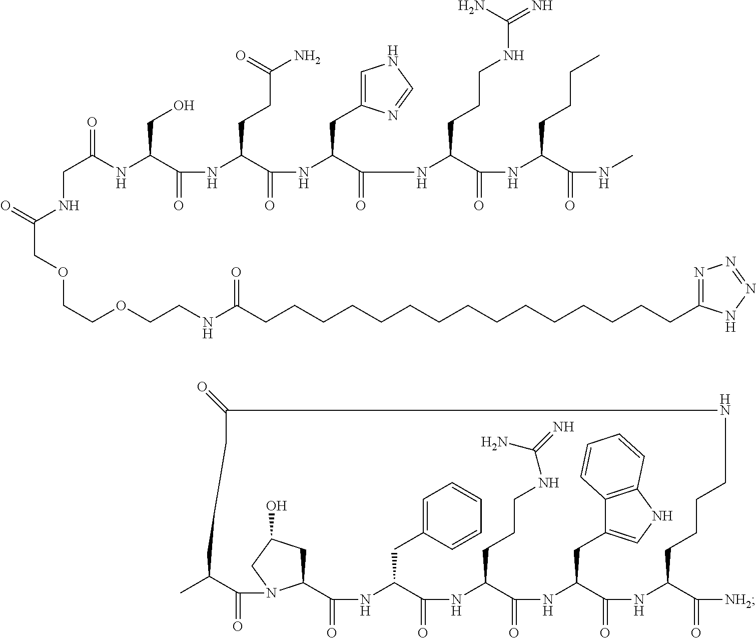

### {2-[2-(16-(Tetrazol-5-yl)hexadecanoylamino)ethoxy]ethoxy}acetyl-Gly-Ser-Gln-His-2-PyAla-Nle-c[Glu-Hyp-D-Phe-Arg-Trp-Lys]-NH₂

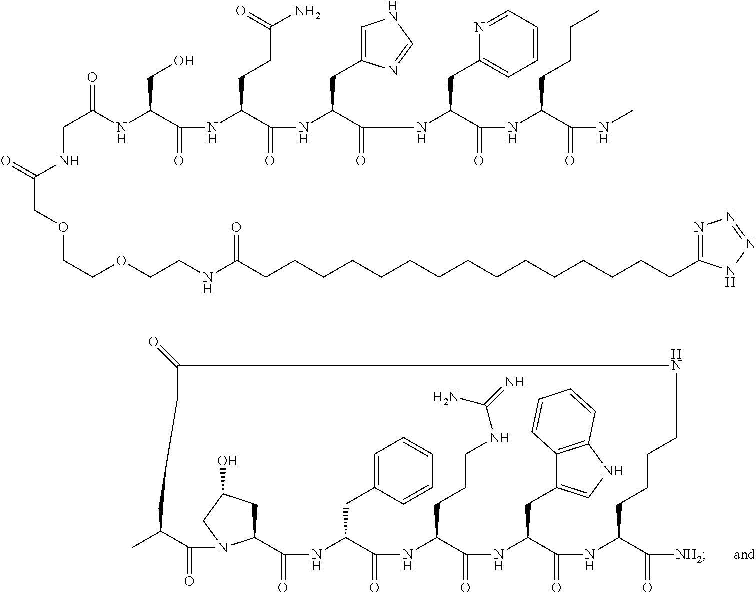

### {2-[2-(16-(Tetrazol-5-yl)hexadecanoylamino)ethoxy]ethoxy}acetyl-Gly-Ser-Gln-His-4-PyAla-Nle-c[Glu-Hyp-D-Phe-Arg-Trp-Lys]-NH₂

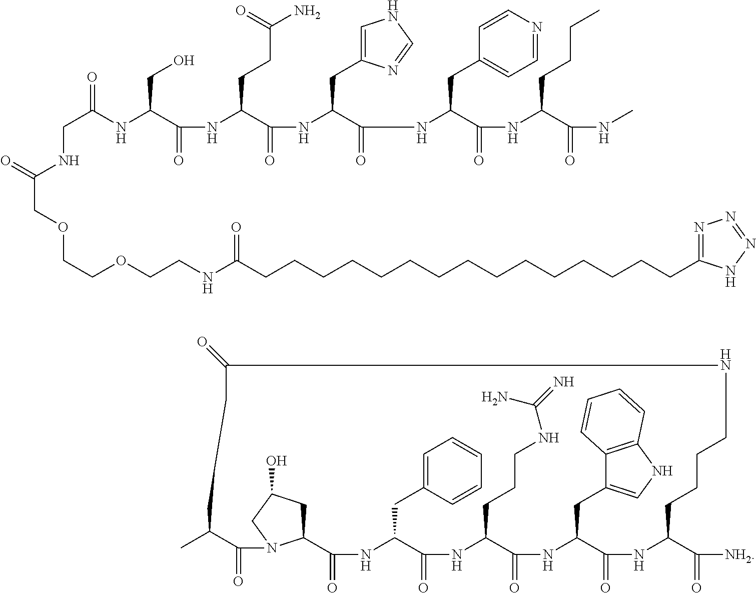

The present invention also encompasses the use of combinations of two or more embodiments of compounds as outlined above.

Examples of frequently used bariatric surgical techniques of relevance in relation to the present invention include, but are not limited to, the following:

vertical banded gastroplasty (also known as “stomach stapling”), wherein a part of the stomach is stapled to create a smaller pre-stomach pouch which serves as a new stomach;

gastric banding, e.g. using an adjustable gastric band system (such as the Swedish Adjustable Gastric Band (SAGB), the LAP-BAND™ or the MIDband™), wherein a small pre-stomach pouch which is to serve as a new stomach is created using an elastomeric (e.g. silicone) band which can be adjusted in size by the patient; and

gastric bypass surgery, e.g. “Roux-en-Y” bypass wherein a small stomach pouch is created using a stapler device and is connected to the distal small intestine, the upper part of the small intestine being reattached in a Y-shaped configuration.

Another technique which is within the scope of the term “bariatric surgery” and variants thereof (e.g. “weight-loss surgery”, “weight-loss surgical intervention” “weight-loss surgical procedure”, “bariatric surgical intervention”, “bariatric surgical procedure” and the like) as employed in the context of the present invention is gastric balloon surgery, wherein an inflatable device resembling a balloon is introduced into the stomach and then inflated, the purpose being to reduce the accessible volume within the stomach to create a sensation of satiety in the patient at an earlier stage than normal and thereby cause a reduction in food intake by the patient.

All of the above-mentioned techniques are in principle reversible. Non-limiting examples of additional, irreversible and consequently generally less frequently employed techniques of relevance in the present context include biliopancreatic diversion and sleeve gastrectomy (the latter of which may also be employed in conjunction with duodenal switch), both of which entail surgical resection of a substantial portion of the stomach.

The administration of a compound of the type in question (optionally in combination with one or more additional therapeutically active compounds or substances as disclosed herein) may take place for a period prior to carrying out the bariatric surgical intervention in question and/or for a period of time subsequent thereto. In many cases it may be preferable to begin administration of a compound of the invention after bariatric surgical intervention has taken place.

In one aspect of the present invention, the compound of the invention is an agonist of a melanocortin receptor, notably an agonist of MC4. In another aspect of the invention, the compound is a selective agonist of MC4. In this context, selectivity is to be understood in relation to the activity of the compound with respect to MC1, MC3 and/or MC5. If a compound is a significantly more potent as a MC4 agonist than as a MC1, MC3 and/or MC5 agonist, it is deemed to be a selective MC4 agonist. The binding affinity of a compound with respect to MC1, MC3, MC5 and MC4 may be determined by comparing the IC50 from an MC1, MC3 or MC5 binding assay as described below under “Assay IV” (MC1), “Assay VIII” (MC3) and “Assay IX” (MC5), respectively, with IC50 from an MC4 binding assay as described below under “Assay V” (MC4). If a compound is more than 10 times, such as more than 50 times, e.g. more than 100 times more potent with respect to MC4 than with respect to MC1, it is deemed to be a selective MC4 agonist with respect to MC1. The agonistic potency of a compound with respect to MC3, MC4 and MC5 may be determined in functional assays as described in “Assay II” (MC 3 and MC5), “Assay X” (MC3) and “Assay III” (MC4). If a compound is more than 10 times, such as more than 50 times, e.g. more than 100 times more potent with respect to MC4 than with respect to MC3, it is deemed to be a selective MC4 agonist with respect to MC3. If a compound is more than 10 times, such as more than 50 times, e.g. more than 100 times more potent with respect to MC4 than with respect to MC5, it is deemed to be a selective MC4 agonist with respect to MC5. In a particular aspect, the compound of the present invention is a selective MC4 agonist with respect to MC1, with respect to MC3, with respect to MC5, with respect to MC1 and MC3, with respect to MC1 and MC5, with respect to MC3 and MC5 or with respect to MC1, MC3 and MC5.

In another aspect of the present invention, the compound of the invention is a selective MC4 agonist and a MC3 antagonist. In this context, a compound is deemed to be a selective MC4 agonist and a MC3 antagonist if it is a selective MC4 agonist with respect to MC1 and MC5 as discussed above, and it antagonizes MC3 as determined as described in “Assay II”. In the latter assay, a compound exhibiting an IC₅₀ value of less than 100 nM, such as less than 10 nM, e.g. less than 5 nM, such as less than 1 nM, is deemed to be a MC3 antagonist.

In a further aspect of the present invention, the compound of the present invention is both a selective MC3 agonist and a selective MC4 agonist. In this context, a compound is deemed to be a selective MC3 and MC4 agonist if it is significantly more potent as an agonist towards MC3 and MC4 than as an agonist toward MC1 and MC5. The selectivity of a compound with respect to MC1 and MC3 may be determined by comparing the binding affinity determined for MC1 as described in “Assay IV” with the binding affinity for MC3 determined as described in “Assay VIII”. If the binding affinity of a compound is more than 10 times, such as more than 50 times, e.g. more than 100 times greater with respect to MC3 than with respect to MC1, it is deemed to be a selective MC3 agonist with respect to MC1. The selectivity of a compound with respect to MC3 and MC5 may be determined by comparing the potency determined as described in “Assay II”. If a compound is more than 10 times, such as more the 50 times, e.g. more than 100 times more potent with respect to MC3 than with respect to MC5, it is deemed to be a selective MC3 agonist with respect to MC5. The MC4 selectivity of a compound with respect to MC3 and MC5 is determined as discussed above.

Compounds of the present invention may exert a protracted effect, i.e. the period of time in which they exert a biological activity is prolonged. Effect is defined as being protracted when a compound significantly reduces food intake in the period from 24 hours to 48 hours in test animals compared to the food intake in the same time period in the vehicle-treated control group of animals in “Assay I”. Alternatively, a protracting effect may be evaluated in an indirect albumin-binding assay, in which Ki determined for binding in the presence of ovalbumin is compared with the the EC₅₀ value determined in the presence of HSA [see Assay VII in the PHARMACOLOGICAL METHODS section (vide infra) for a description of a suitable assay procedure].

Compounds of the type in question modulate melanocortin receptors, and they are therefore believed to be particularly suited for the treatment of diseases or states which can be treated by a modulation of melanocortin receptor activity. In particular, compounds of the type in question are believed to be suited for the treatment of diseases or states via activation of MC4.

In one aspect, the present invention relates to a method of treating obesity or preventing overweight, the method comprising administration to a patient in need thereof of an effective amount of a compound of the type in question (as disclosed and detailed above) in combination with surgical intervention (bariatric surgery) in the patient for the purpose of achieving weight loss or preventing weight gain.

In a further aspect, the present invention provides a method of regulating appetite, the method comprising administration to a patient in need thereof of an effective amount of a compound of the type in question (as disclosed and detailed above) in combination with surgical intervention (bariatric surgery) in the patient for the purpose of achieving weight loss or preventing weight gain.

Another aspect of the invention relates to a method of inducing satiety, the method comprising administration to a patient in need thereof of an effective amount of a compound of the type in question (as disclosed and detailed above) in combination with surgical intervention (bariatric surgery) in the patient for the purpose of achieving weight loss or preventing weight gain.

Still further aspects of the invention include the following:

a method of treating a disease or state related to overweight or obesity, the method comprising administration to a patient in need thereof of an effective amount of a compound of the type in question in combination with surgical intervention in the patient for the purpose of achieving weight loss or preventing weight gain.

a method of treating bulimia, the method comprising administration to a patient in need thereof of an effective amount of a compound of the type in question in combination with surgical intervention in the patient for the purpose of achieving weight loss or preventing weight gain.

a method of treating a disease or state selected from atherosclerosis, hypertension, diabetes, type 2 diabetes, impaired glucose tolerance (IGT), dyslipidemia, coronary heart disease, gallbladder disease, gall stone, osteoarthritis, cancer, sexual dysfunction and risk of premature death, the method comprising administration to a patient in need thereof of an effective amount of a compound of the type in question in combination with surgical intervention in the patient for the purpose of achieving weight loss or preventing weight gain.

In particular, the methodology of the present invention may be suited for the treatment of diseases in obese or overweight patients. Accordingly, the present invention also provides a method of treating, in an obese patient, a disease or state selected from type 2 diabetes, impaired glucose tolerance (IGT), dyslipidemia, coronary heart disease, gallbladder disease, gall stone, osteoarthritis, cancer, sexual dysfunction and risk of premature death in obese patients, the method comprising administration to a patient in need thereof of an effective amount of a compound of the type in question in combination with surgical intervention in the patient for the purpose of achieving weight loss or preventing weight gain.

In addition, MC4 agonists may have a positive effect on insulin sensitivity, on drug abuse by modulating the reward system and on hemorrhagic shock. Furthermore, MC3 and MC4 agonists have antipyretic effects, and both have been suggested to be involved in peripheral nerve regeneration. MC4 agonists are also known to reduce stress response. In addition to treating drug abuse, treating or preventing hemorrhagic shock, and reducing stress response, compounds of the type in question may also be of value in treating alcohol abuse, treating stroke, treating ischemia and protecting against neuronal damage.

In all of the therapeutic methods or indications disclosed above, the compound in question may be administered alone. However, it may also be administered in combination with one or more additional therapeutically active agents, substances or compounds, either sequentially or concomitantly.

A typical dosage of a compound of the type in question when employed in a method according to the present invention is in the range of from about 0.001 to about 100 mg/kg body weight per day, preferably from about 0.01 to about 50 mg/kg body weight per day, such as from about 0.05 to about 10 mg/kg body weight per day, administered in one or more doses, such as from 1 to 3 doses. The exact dosage will depend upon the frequency and mode of administration, the sex, age, weight and general condition of the subject treated, the nature and severity of the condition treated, any concomitant diseases to be treated and other factors evident to those skilled in the art.

Compounds of the type in question may conveniently be formulated in unit dosage form using techniques well known to those skilled in the art. A typical unit dosage form intended for oral administration one or more times per day, such as from one to three times per day, may suitably contain from 0.05 to about 1000 mg, preferably from about 0.1 to about 500 mg, such as from about 0.5 mg to about 200 mg of a compound of the invention.

Compounds of the type in question comprise compounds that are believed to be well-suited to administration with longer intervals than, for example, once daily, Thus, appropriately formulated compounds may be suitable for, e.g., twice-weekly or once-weekly administration by a suitable route of administration, such as one of the routes disclosed herein.

As described above, compounds of the type in question may be administered or applied in combination with one or more additional therapeutically active compounds or substances. Suitable additional compounds or substances may be selected, for example, from antidiabetic agents, antihyperlipidemic agents, antiobesity agents, antihypertensive agents and agents for the treatment of complications resulting from, or associated with, diabetes.

Suitable antidiabetic agents include insulin, insulin derivatives or analogues, GLP-1 (glucagon like peptide-1) derivatives or analogues [such as those disclosed in WO 98/08871 (Novo Nordisk A/S), which is incorporated herein by reference, or other GLP-1 analogues such as Byetta (exenatide; Eli Lilly/Amylin)], amylin, amylin analogues (such as Symlin™/Pramlintide), as well as orally active hypoglycemic agents.

Suitable orally active hypoglycemic agents include: imidazolines; sulfonylureas; biguanides; meglitinides; oxadiazolidinediones; thiazolidinediones; insulin sensitizers; α-glucosidase inhibitors; agents acting on the ATP-dependent potassium channel of the pancreatic β-cells, e.g. potassium channel openers such as those disclosed in WO 97/26265, WO 99/03861 and WO 00/37474 (Novo Nordisk A/S) which are incorporated herein by reference; potassium channel openers such as ormitiglinide; potassium channel blockers such as nateglinide or BTS-67582; glucagon antagonists such as those disclosed in WO 99/01423 and WO 00/39088 (Novo Nordisk A/S and Agouron Pharmaceuticals, Inc.), all of which are incorporated herein by reference; GLP-1 agonists such as those disclosed in WO 00/42026 (Novo Nordisk A/S and Agouron Pharmaceuticals, Inc.), which are incorporated herein by reference; amylin agonists; DPP-IV (dipeptidyl peptidase-IV) inhibitors; PTPase (protein tyrosine phosphatase) inhibitors; glucokinase activators, such as those described in WO 02/08209 to Hoffmann La Roche; inhibitors of hepatic enzymes involved in stimulation of gluconeogenesis and/or glycogenolysis; glucose uptake modulators; GSK-3 (glycogen synthase kinase-3) inhibitors; compounds modifying lipid metabolism, such as antihyperlipidemic agents and antilipidemic agents; compounds lowering food intake; as well as PPAR (peroxisome proliferator-activated receptor) agonists and RXR (retinoid X receptor) agonists such as ALRT-268, LG-1268 or LG-1069.

Other examples of suitable additional therapeutically active substances include insulin or insulin analogues; sulfonylureas, e.g. tolbutamide, chlorpropamide, tolazamide, glibenclamide, glipizide, glimepiride, glicazide or glyburide; biguanides, e.g. metformin; and meglitinides, e.g. repaglinide or senaglinide/nateglinide.

Further examples of suitable additional therapeutically active substances include thiazolidinedione insulin sensitizers, e.g. troglitazone, ciglitazone, pioglitazone, rosiglitazone, isaglitazone, darglitazone, englitazone, CS-011/CI-1037 or T 174, or the compounds disclosed in WO 97/41097 (DRF-2344), WO 97/41119, WO 97/41120, WO 00/41121 and WO 98/45292 (Dr. Reddy's Research Foundation), the contents of all of which are incorporated herein by reference.

Additional examples of suitable additional therapeutically active substances include insulin sensitizers, e.g. GI 262570, YM-440, MCC-555, JTT-501, AR-H039242, KRP-297, GW-409544, CRE-16336, AR-H049020, LY510929, MBX-102, CLX-0940, GW-501516 and the compounds disclosed in WO 99/19313 (NN622/DRF-2725), WO 00/50414, WO 00/63191, WO 00/63192 and WO 00/63193 (Dr. Reddy's Research Foundation), and in WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), the contents of all of which are incorporated herein by reference.

Still further examples of suitable additional therapeutically active substances include:

α-glucosidase inhibitors, e.g. voglibose, emiglitate, miglitol or acarbose;

glycogen phosphorylase inhibitors, e.g. the compounds described in WO 97/09040 (Novo Nordisk A/S);

glucokinase activators;

agents acting on the ATP-dependent potassium channel of the pancreatic β-cells, e.g. tolbutamide, glibenclamide, glipizide, glicazide, BTS-67582 or repaglinide;

Other suitable additional therapeutically active substances include antihyperlipidemic agents and antilipidemic agents, e.g. cholestyramine, colestipol, clofibrate, gemfibrozil, lovastatin, pravastatin, simvastatin, probucol or dextrothyroxine.

Further agents which are suitable as additional therapeutically active substances include antiobesity agents and appetite-regulating agents. Such substances may be selected from the group consisting of CART (cocaine amphetamine regulated transcript) agonists, NPY (neuropeptide Y) antagonists, MC3 (melanocortin receptor 3) agonists, MC3 antagonists, MC4 (melanocortin receptor 4) agonists, orexin antagonists, TNF (tumor necrosis factor) agonists, CRF (corticotropin releasing factor) agonists, CRF BP (corticotropin releasing factor binding protein) antagonists, urocortin agonists, 3 adrenergic agonists such as CL-316243, AJ-9677, GW-0604, LY362884, LY377267 or AZ-40140, MC1 (melanocortin receptor 1) agonists, MCH (melanocyte-concentrating hormone) antagonists, CCK (cholecystokinin) agonists, serotonin reuptake inhibitors (e.g. fluoxetine, seroxat or citalopram), serotonin and norepinephrine reuptake inhibitors, 5HT (serotonin) agonists, bombesin agonists, galanin antagonists, growth hormone, growth factors such as prolactin or placental lactogen, growth hormone releasing compounds, TRH (thyrotropin releasing hormone) agonists, UCP 2 or 3 (uncoupling protein 2 or 3) modulators, chemical uncouplers, leptin agonists, DA (dopamine) agonists (bromocriptin, doprexin), lipase/amylase inhibitors, PPAR modulators, RXR modulators, TR β agonists, adrenergic CNS stimulating agents, AGRP (agouti-related protein) inhibitors, histamine H3 receptor antagonists such as those disclosed in WO 00/42023, WO 00/63208 and WO 00/64884, the contents of all of which are incorporated herein by reference, exendin-4, GLP-1 agonists, ciliary neurotrophic factor, amylin analogues, peptide YY₃₋₃₆ (PYY3-36) (Batterham et al, Nature 418, 650-654 (2002)), PYY3-36 analogues, NPY Y2 receptor agonists, NPY Y4 receptor agonists and substances acting as combined NPY Y2 and NPY Y4 agonists.

Further suitable antiobesity agents are bupropion (antidepressant), topiramate (anticonvulsant), ecopipam (dopamine D1/D5 antagonist) and naltrexone (opioid antagonist).

Among embodiments of suitable antiobesity agents for use in a method of the invention as additional therapeutically active substances in combination with a compound of the invention are leptin and analogues or derivatives of leptin.

Additional embodiments of suitable antiobesity agents are serotonin and norepinephrine reuptake inhibitors, e.g. sibutramine.

Other embodiments of suitable antiobesity agents are lipase inhibitors, e.g. orlistat.

Still further embodiments of suitable antiobesity agents are adrenergic CNS stimulating agents, e.g. dexamphetamine, amphetamine, phentermine, mazindol, phendimetrazine, diethylpropion, fenfluramine or dexfenfluramine.

Other examples of suitable additional therapeutically active compounds include antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin.

In certain embodiments of the present invention, the compound in question may be administered or applied in combination with more than one of the above-mentioned, suitable additional therapeutically active compounds or substances, e.g. in combination with: metformin and a sulfonylurea such as glyburide; a sulfonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

### Pharmaceutical Compositions

Appropriate embodiments of formulations of a compound of the type in question will often contain the compound in a concentration of from 10⁻³ mg/ml to 200 mg/ml, such as, e.g., from 10⁻¹ mg/ml to 100 mg/ml. The pH in such a formulation of the invention will typically be in the range of 2.0 to 10.0. The formulation may further comprise a buffer system, preservative(s), tonicity agent(s), chelating agent(s), stabilizer(s) and/or surfactant(s). In one embodiment of the invention the pharmaceutical formulation is an aqueous formulation, i.e. formulation comprising water, and the term “aqueous formulation” in the present context may normally be taken to indicate a formulation comprising at least 50% by weight (w/w) of water. Such a formulation is typically a solution or a suspension. An aqueous formulation of the invention in the form of an aqueous solution will normally comprise at least 50% (w/w) of water. Likewise, an aqueous formulation of the invention in the form of an aqueous suspension will normally comprise at least 50% (w/w) of water.

A suitable pharmaceutical composition (formulation) of a compound of the type in question may be a freeze-dried (i.e. lyophilized) formulation intended for reconstitution by the physician or the patient via addition of solvents and/or diluents prior to use.

In addition, a pharmaceutical composition (formulation) of a compound of the type in question may be a dried formulation (e.g. freeze-dried or spray-dried) ready for use without any prior dissolution.

A suitable pharmaceutical composition (formulation) will comprise an aqueous solution of a compound of the type in question and a buffer, the compound being present in a concentration of 0.1-100 mg/ml or above, and the formulation having a pH from about 2.0 to about 10.0.

Administration of pharmaceutical compositions of compounds of the type in question to patients in need thereof may be via several routes of administration. These include, for example, lingual, sublingual, buccal, in the mouth, oral, in the stomach and intestine, nasal, pulmonary (for example through the bronchioles and alveoli or a combination thereof), epidermal, dermal, transdermal, vaginal, rectal, ocular (for example through the conjunctiva), uretal and parenteral.

Parenteral administration may be performed by subcutaneous, intramuscular, intraperitoneal or intravenous injection by means of a syringe, for example a syringe in the form of a pen device. Alternatively, parenteral administration can be performed by means of an infusion pump. A further option is administration of a composition which is a liquid (typically aqueous) solution or suspension in the form of a nasal or pulmonary spray. As a still further option, a pharmaceutical composition can be adapted to transdermal administration (e.g. by needle-free injection or via a patch, such as an iontophoretic patch) or transmucosal (e.g. buccal) administration.

All references, including publications, patent applications and patents, cited herein are hereby incorporated by reference in their entirety and to the same extent as if each reference were individually and specifically indicated to be incorporated by reference and were set forth in its entirety herein (to the maximum extent permitted by law).

Headings and sub-headings are used herein for convenience only, and should not be construed as limiting the invention in any way.

The use of any and all examples, or exemplary language (including “for instance”, “for example”, “e.g.” and “such as”) in the present specification is intended merely to better illuminate the invention, and does not pose a limitation on the scope of the invention unless otherwise indicated. No language in the specification should be construed as indicating any non-claimed element as being essential to the practice of the invention.

The citation and incorporation of patent documents herein is done for convenience only, and does not reflect any view of the validity, patentability and/or enforceability of such patent documents.

The present invention includes all modifications and equivalents of the subject matter recited in the claims appended hereto, as permitted by applicable law.

### Examples

### List of Abbreviations Employed

AcOH acetic acid
BSA bovine serum albumin
DBU 1,8-diazabicyclo[5.4.0]undec-7-ene (1,5-5)
DCM dichloromethane
DIC diisopropylcarbodiimide
DIPEA ethyldiisopropylamine
DMAP 4-N,N-dimethylaminopyridine
DMEM Dulbecco's Modified Eagle Medium
DMF N,N-dimethylformamide
DMSO dimethyl sulfoxide
EGTA 1,2-di(2-aminoethoxy)ethane-N,N,N′,N′-tetraacetic acid
FCS fetal calf serum
Fmoc 9-fluorenylmethyloxycarbonyl
HEPES 2-[4-(2-hydroxyethyl)-piperazin-1-yl]-ethanesulfonic acid
HOAt 1-hydroxy-7-azabenzotriazole
HOBt 1-hydroxybenzotriazole
HSA human serum albumin
IBMX 3-isobutyl-1-methylxanthine
MC1 melanocortin receptor subtype 1 (also denoted melanocortin receptor 1)
MC2 melanocortin receptor subtype 2 (also denoted melanocortin receptor 2)
MC3 melanocortin receptor subtype 3 (also denoted melanocortin receptor 3)
MC4 melanocortin receptor subtype 4 (also denoted melanocortin receptor 4)
MC5 melanocortin receptor subtype 5 (also denoted melanocortin receptor 5)
MeCN acetonitrile
MeOH methanol
min minutes
α-MSH α-form of melanocyte-stimulating hormone
MTX methotrexate
NEt₃ triethylamine
NMP N-methylpyrrolidone
PBS phosphate-buffered saline
PEI polyethyleneimine
pen/strep penicillin/streptomycin
PyBOP (benzotriazol-1-yloxy)tripyrrolidino-phosphonium hexafluorophosphate

All compounds of the type in question can be synthesized by those skilled in the art using standard coupling and deprotection steps. A description of all necessary tools and synthetic methods including standard abbreviations for peptide synthesis can be found in “The Fine Art Of Solid Phase Synthesis”, 2002/3 Catalogue, Novabiochem. Non-standard procedures and syntheses of special building blocks are described below.

In the examples listed below, Rt values are retention times and the mass values are those detected by the mass spectroscopy (MS) detector and obtained using one of the following HPLC-MS devices (LCMS).

### LCMS (System 1)

Agilent 1100 Series, electrospray; column: Waters XTerra® C₁₈ 5 μm 3.0×50 mm; water/acetonitrile containing 0.05% TFA; gradient: 5%→100% acetonitrile from 0 to 6.75 min, elution until t=9.0 min; flow 1.5 ml/min.

### LCMS (System 2)

Sciex API-150 Ex Quadrupole MS, electrospray, m/z=200 to m/z=1500; column: Waters XTerra® MS C₁₈ 5 μm 3.0×50 mm; elution with a mixture of solution A (water containing 0.1% TFA) and solution B (acetonitrile containing 0.08% TFA); gradient: 5%→20% solution B from 1.0 to 3.0 min, 20%→50% solution B from 3.0 to 16.0 min, 50%→90% solution B from 16.0 to 18.0 min, elution until t=18.0 min; flow 1.5 ml/min.

### LCMS (System 3)

Sciex API-100 Quadrupole MS, electrospray, m/z=300 to m/z=2000; column: Waters XTerra® MS C₁₈ 5 μm 3.0×50 mm; water/acetonitrile containing 0.05% TFA; gradient: 5%→90% acetonitrile from 0 to 7.5 min; flow 1.5 ml/min.

### MALDI-MS

Molecular weights of the peptides were determined using matrix-assisted laser desorption ionization time of flight mass spectroscopy (MALDI-MS), recorded on a Voyager-DE (Perseptive Biosystems). A matrix of sinapinic acid (3,5-dimethoxy-4-hydroxycinnamic acid) was used.

A typical example of a synthesis procedure which includes a cyclization step is as follows:

### Example 1

### 16-(Tetrazol-5-yl)hexadecanoyl-Gly-Thr-Gln-His-Ser-Nle-c[Glu-Hyp-D-Phe-Arg-Trp-Lys]-NH₂

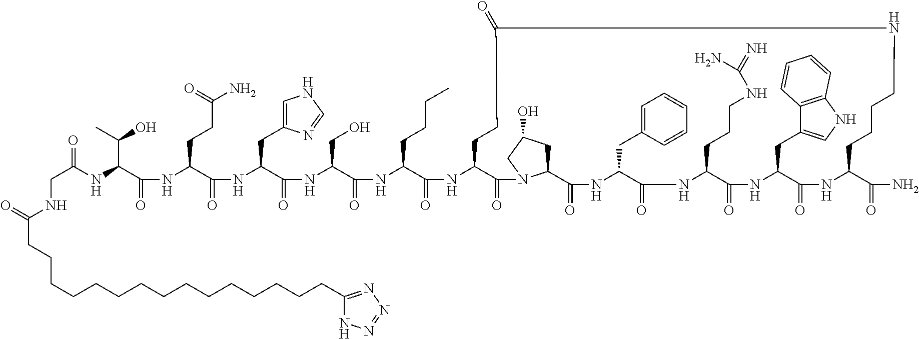

### Step A for Example 1: Protected Peptide Resin Fmoc-c[Glu-Hyp(tBu)-D-Phe-Arg(Pbf)-Trp-Lys]-NH-Rink linker-polystyrene

Fmoc-Rink resin (4-(2′,4′-dimethoxyphenyl-Fmoc-aminomethyl)-phenoxypolystyrene resin, Bachem D-2080, Lot 514460; 0.47 mmol/g) was filled into two 60 ml Teflon reactors with frit (per reactor: 4.256 g, 2.0 mmol). The resin in each reactor was washed with 35 ml DCM.

Removal of Fmoc: The resin was shaken with a solution of 20% piperidine in NMP (30 ml) for 20 min and then washed with NMP/DCM 1:1 (5×30 ml).

Acylation with Fmoc-Lys(Mtt)-OH: In a separate glass vial, the Fmoc-amino acid (12.0 mmol) was mixed with NMP (15 ml), DCM (27 ml) and a 1M solution (12.0 ml, 12.0 mmol) of 1-hydroxybenzotriazol (HOBt) in NMP. To the resulting clear solution, DIC (1.872 ml, 12.0 mmol) was quickly added and the solution was shaken immediately thereafter. The solution was left to stand in a closed vial for 30 min. 30 ml (6.0 mmol HOBt ester) of this solution was added to each reactor and the resin was shaken for 2½ hours. Ethyldiisopropylamine (DIPEA) (per reactor 0.514 ml, 2.0 mmol) was added and the mixture was shaken for 18 h. The resin was washed with NMP/DCM 1:1 (4×30 ml).

Removal of Fmoc: As described above.

Acylation with Fmoc-TrD(Boc)-OH: In a separate glass vial, the Fmoc-amino acid (12.0 mmol) was mixed with NMP (15 ml), DCM (27 ml) and 1M HOBt-NMP solution (12.0 ml, 12.0 mmol). To the resulting clear solution, DIC (1.872 ml, 12.0 mmol) was quickly added and the solution was shaken immediately thereafter. The solution was left to stand in a closed vial for 30 min. 30 ml (6.0 mmol HOBt ester) of this solution was added to each reactor and the resin was shaken for 21 h. The liquids were filtered off and the resin was washed with NMP/DCM 1:1 (4×30 ml).

In a similar manner, the following amino acids were successively attached to the resin: Fmoc-Arg(Pbf)-OH, Fmoc-D-Phe-OH, Fmoc-Hyp(tBu)-OH and Fmoc-Glu(2-phenylisopropyloxy)-OH. Coupling with Fmoc-Glu(2-phenylisopropyloxy)-OH was performed by using HOAt instead of HOBt, and DIPEA (2.0 mmol per reactor added after HOAt ester formation). The resulting Fmoc-protected resin was extensively washed with DCM.

Selective side-chain deprotection of Lys and Glu: The resin was shaken with a solution of 2% TFA and 3% triisopropylsilane in DCM (30 ml) for 10 min and the liquid was filtered off. This procedure was repeated another eight times. The resin was washed with DCM (4×30 ml), 10% DIPEA in DCM (2×30 ml) and DCM (2×30 ml).

Side-chain cyclisation of Lys with Glu: In a separate glass vial, PyBOP (6.246 g=12.0 mmol) was mixed with 1M HOBt-NMP solution (12.0 ml=12.0 mmol), DCM (30 ml) and NMP (18 ml). 30 ml (containing 6.0 mmol PyBOP/HOBt) of this solution was added to each reactor, followed by DIPEA (2.054 ml=12.0 mmol). The resin was shaken for 18 h. The liquids were filtered off and the resin was washed with NMP/DCM 1:1 (4×30 ml).

Capping of non-acylated amino groups: Each resin was shaken with a solution of Boc anhydride (12 mmol per reactor) in DCM (30 ml per reactor) for 1 h. The liquids were filtered off and the resin was washed with DCM (3×30 ml), DCM/MeOH 2:1 (2×30 ml), THF (4×30 ml) and DCM (3×30 ml).

This afforded 13.92 g of resin, corresponding to a supposed maximum loading of 0.29 mmol/g if complete reactions are assumed.

### Step B for Example 1: 16-(tetrazol-5-yl)hexadecanoyl-Gly-Thr-GIn-His-Ser-Nle-c[Glu-Hyp-D-Phe-Arg-Trp-Lys]-NH₂

A 10 ml Teflon reactor with frit was charged with resin Fmoc-c[Glu-Hyp(tBu)-D-Phe-Arg(Pbf)-Trp-Lys]-NH-Rink linker-polystyrene (0.345 g, theoretically 0.10 mmol, available by Step A described above). The resin was washed with DCM (3 ml).

Removal of Fmoc: The resin was shaken with a solution of 20% piperidine in NMP (3.5 ml) for 20 min and then washed with NMP/DCM 1:1 (6×4 ml).

Acylation with Fmoc-Nle-OH: In a separate glass vial, the Fmoc-amino acid (0.5 mmol) was mixed with NMP (0.65 ml), DCM (1.15 ml) and 1M HOBt-NMP solution (0.5 ml, 0.5 mmol). To the resulting clear solution, DIC (0.078 ml, 0.5 mmol) was quickly added and the solution was shaken immediately thereafter. The solution was left to stand in a closed vial for 30 min and then added to the resin. The mixture was shaken for 105 min. The liquids were filtered off and the resin was washed with NMP/DCM 1:1 (4×4 ml).

In a similar manner, the following carboxylic acids were successively attached to the resin: Fmoc-Ser(tBu)-OH, Fmoc-His(Trt)-OH, Fmoc-Gln(Trt)-OH, Fmoc-Thr(tBu)-OH, Fmoc-Gly-OH and 16-(tetrazol-5-yl)hexadecanoic acid (available by the synthetic procedure described below). Finally, the resin was washed with NMP/DCM 1:1 (6×3 ml), DCM/MeOH 2:1 (2×3 ml), THF (2×3 ml) and DCM (3×3 ml).

Cleavage from the resin: The resin was shaken with a premixed solution (4 ml) containing TFA (95 vol-%), triisopropylsilane (2.5 vol-% l) and water (2.5 vol-%) for 2 h. The mixture was filtered and the filtrate was collected in a glass vial. The resin was washed with 2×3 ml DCM/TFA 2:1 and the filtrates were collected. The combined filtrate solution was concentrated to give a red oil.

Precipitation with ether: The oily residue was treated with diethyl ether (30 ml) to give a solid precipitate. The ether phase was removed after centrifugation. The solid residue was washed again with diethyl ether (30 ml). After centrifugation and removal of the ether phase, the solid residue was left to stand overnight in order to remove remaining diethyl ether.

Purification: The crude product precipitated from diethyl ether was dissolved in a mixture of acetonitrile (5.5 ml), acetic acid (0.5 ml) and water to give a total volume of about 21 ml. The resulting liquid was filtered and then injected into a Gilson preparative HPLC device. Elution was performed with water/acetonitrile containing 0.1% TFA with a gradient from 29% to 41% acetonitrile. The eluate was collected as fractions of 5 ml (peak fractions) or 12 ml (non-peak fractions), respectively. Relevant fractions were checked by analytical HPLC. Fractions containing the pure target peptide were mixed and concentrated under reduced pressure to give a colourless solution. This was diluted with de-ionised water and treated with 1M aqueous HCl (0.6 ml). The resulting clear solution was dispensed into glass vials. The vials were capped with Millipore glassfibre prefilters. Freeze-drying for three days afforded the peptide hydrochloride (27.8 mg, 16% yield) as a white solid.

Analytical HPLC (Waters Symmetry300 C18, 5 μm, 3.9×150 mm; 42° C.; water/acetonitrile containing 0.05% TFA; gradient: 5%→95% acetonitrile from 0 to 15 min; flow 1 ml/min): t_{R}=8.32 min (100% purity by UV 214 nm) LCMS (system 1): Rt=3.29 min; ((m+2)/2)=896

Examples of further compounds of the invention which may be obtained in a manner analogous to the compound of Example 1 are the compounds of Examples 2-52, below:

### Example 2

### 16-(Tetrazol-5-yl)hexadecanoyl-Gly-Thr-Gln-Dap-Ser-Nle-c[Glu-Hyp-D-Phe-Arg-Trp-Lys]-NH₂

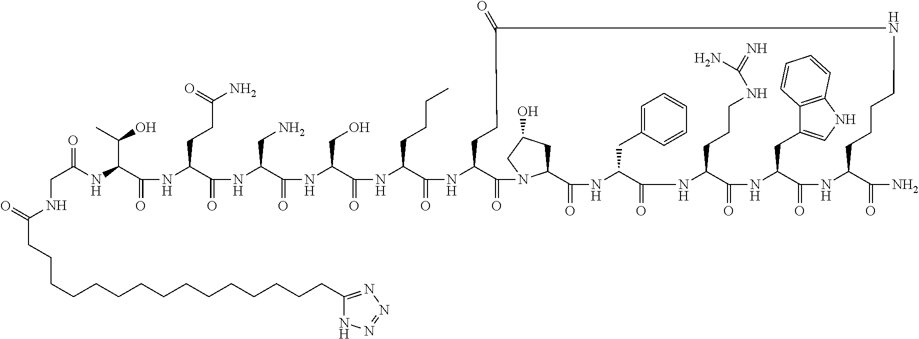

LCMS (system 1): Rt=3.28 min; ((m+2)/2)=870

### Example 3

### {2-[2-(16-(Tetrazol-5-yl)hexadecanoylamino)ethoxy]ethoxy}acetyl-Gly-Thr-Gln-Dap-Ser-Nle-c[Glu-Hyp-D-Phe-Arg-Trp-Lys]-NH₂

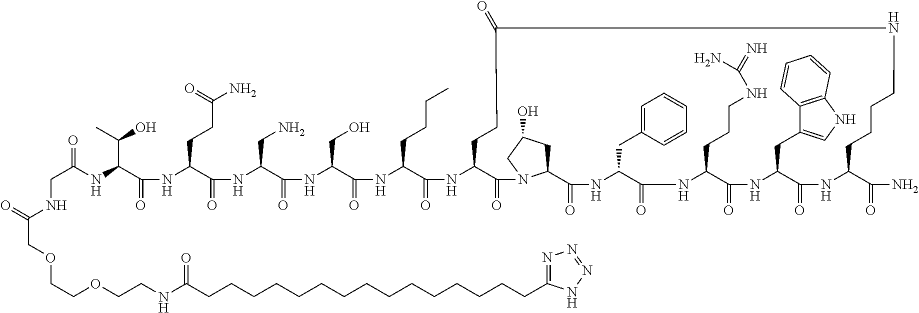

LCMS (system 1): Rt=3.38 min; ((m+2)/2)=943

This compound was prepared using the commercially available building block Fmoc-NH—CH₂—CH₂—O—CH₂—CH₂—O—CH₂—CO₂H.

### Example 4

### {2-[2-(16-(Tetrazol-5-yl)hexadecanoylamino)ethoxy]ethoxy}acetyl-Gly-Ser-Gln-His-Ser-Nle-c[Glu-Dap-D-Phe-Arg-Trp-Lys]-NH₂

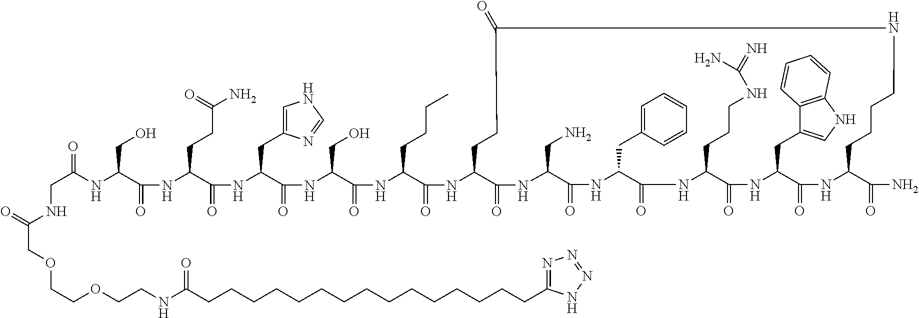

LCMS (system 2): Rt=9.49 min; ((m+2)/2)=947

### Example 5

### {2-[2-(16-(Tetrazol-5-yl)hexadecanoylamino)ethoxy]ethoxy}acetyl-Gly-Ser-Gln-His-Dap-Nle-c[Glu-Hyp-D-Phe-Arg-Trp-Lys]-NH₂

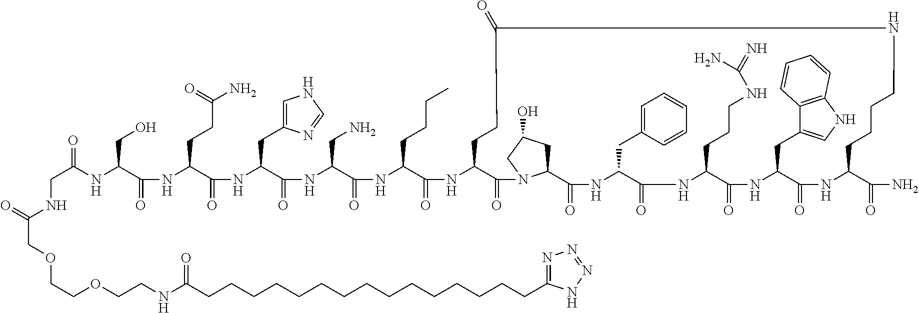

LCMS (system 2): Rt=9.56 min; ((m+2)/2)=961

### Example 6

### 4-(16-(Tetrazol-5-yl)hexadecanoylsulfamoyl)butanoyl-Gly-Ser-D-Gln-His-Dap-Nle-c[Glu-Hyp-D-Phe-Arg-Trp-Lys]-NH₂

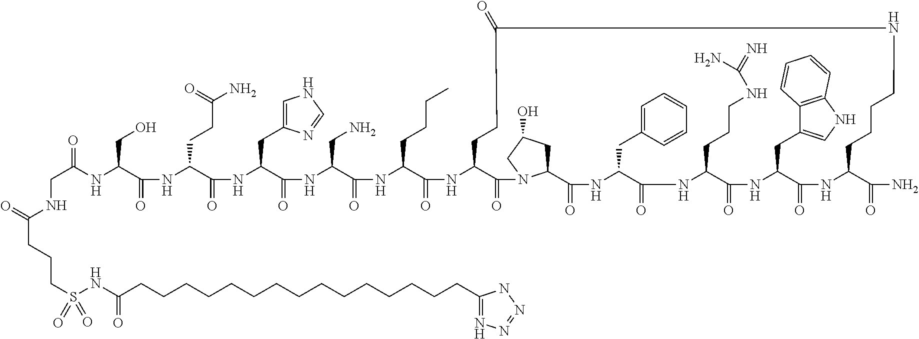

LCMS (system 2): Rt=10.36 min; ((m+2)/2)=962

This compound was prepared using the building block 4-(N-(16-(tetrazol-5-yl)hexadecanoyl)-sulfamoyl)butyric acid. The synthesis of the building block is described below.

### Example 7

### {2-[2-(2-{2-[2-(16-(Tetrazol-5-yl)hexadecanoylamino)ethoxy]ethoxy}acetylamino)ethoxy]-ethoxy}acetyl-Nle-c[Glu-Dap-D-Phe-Arg-Trp-Lys]-NH₂

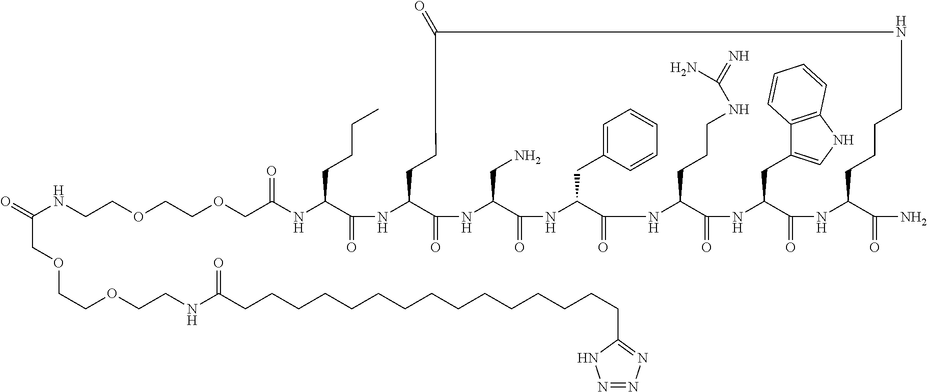

LCMS (system 2): Rt=11.60 min; ((m+2)/2)=772

### Example 8

### (2-{2-[4-(16-(Tetrazol-5-yl)hexadecanoylsulfamoyl)butanoylamino]ethoxy}ethoxy)acetyl-His-Nle-c[Glu-Dap-D-Phe-Arg-Trp-Lys]-NH₂

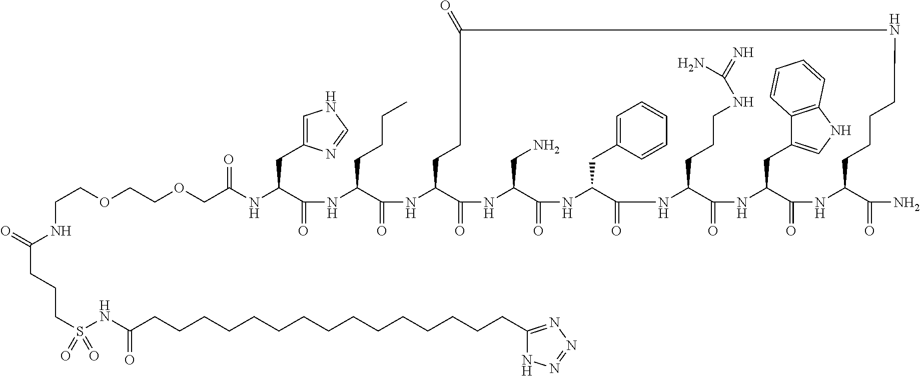

LCMS (system 2): Rt=10.93 min; ((m+2)/2)=842

### Example 9

### {2-[2-(2-{2-[2-(15-Carboxypentadecanoylamino)ethoxy]ethoxy}acetylamino)ethoxy]ethoxy}-acetyl-Pro-Nle-c[Glu-Hyp-D-Phe-Arg-Trp-Lys]-NH₂

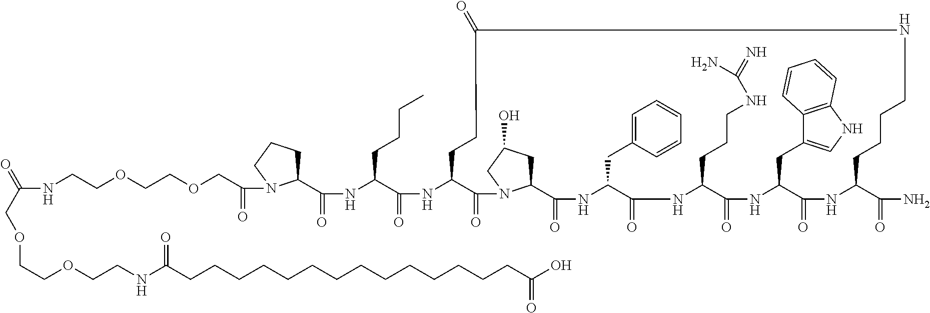

LCMS (system 2): Rt=11.12 min; ((m+2)/2)=815

This compound was prepared using the building block hexadecanedioic acid mono-tert-butyl ester. The synthesis of the building block is outlined below.

### Example 10

### {2-[2-(2-{2-[2-(15-Carboxypentadecanoylamino)ethoxy]ethoxy}acetylamino)ethoxy]ethoxy}acetyl-Nle-c[Glu-Hyp-D-Phe-Arg-Trp-Lys]-NH₂

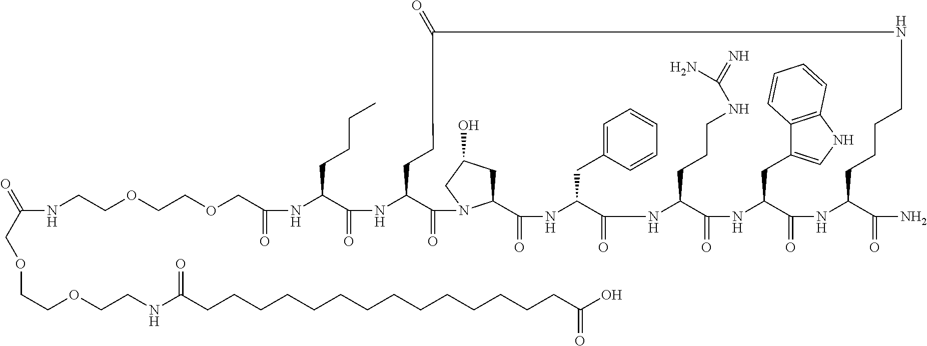

LCMS (system 2): Rt=12.20 min; ((m+2)/2)=766

### Example 11

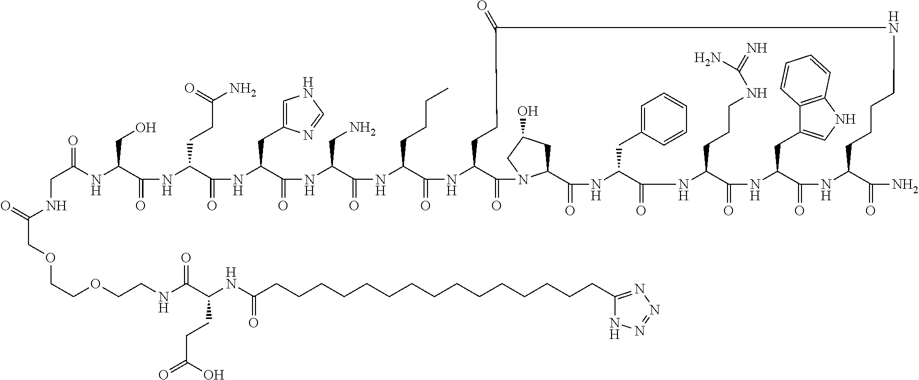

### Example 12

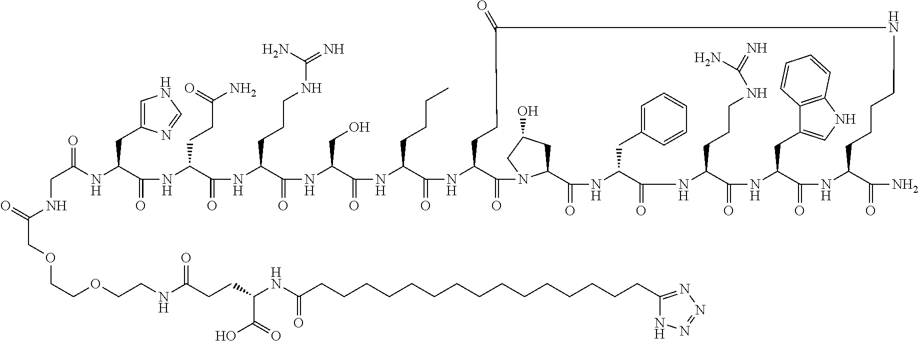

### Example 13

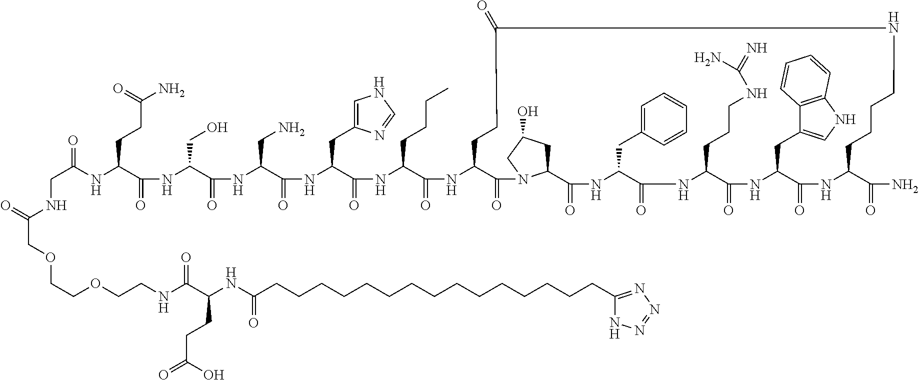

### Example 14

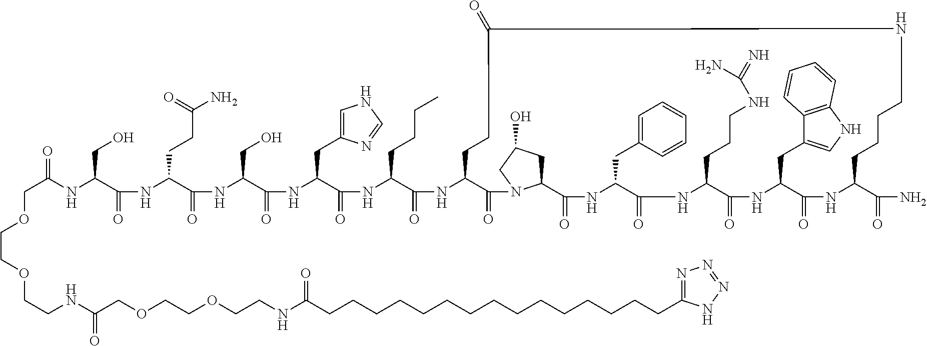

### Example 15

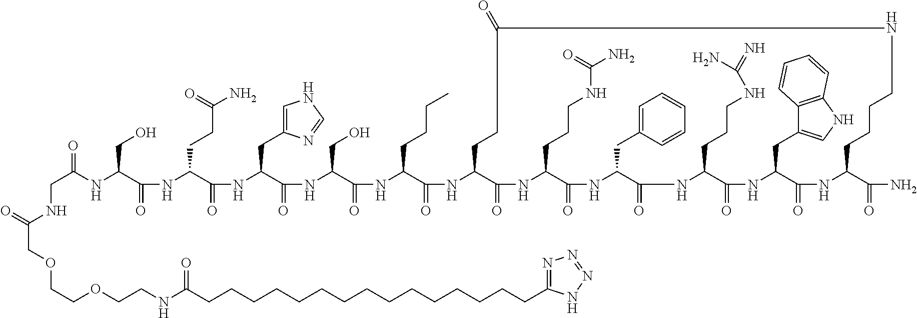

### Example 16

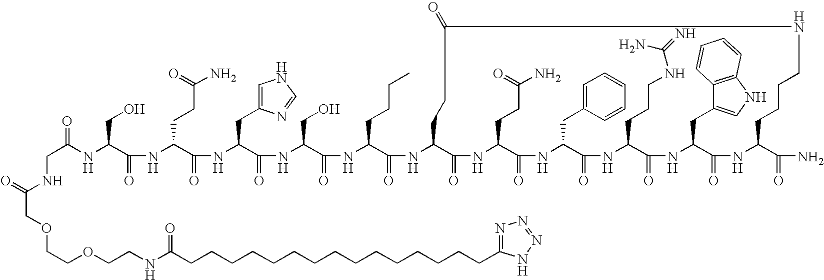

### Example 17

### (2-{2-[2-(2-{2-[(R)-4-Carboxy-2-(16-(1H-tetrazol-5-yl)hexadecanoylamino)butanoylamino]ethoxy}ethoxy)acetylamino]ethoxy}ethoxy)acetyl-Ser-Ser-Nle-c[Glu-Hyp-D-Phe-Arg-Trp-Lys]-NH₂

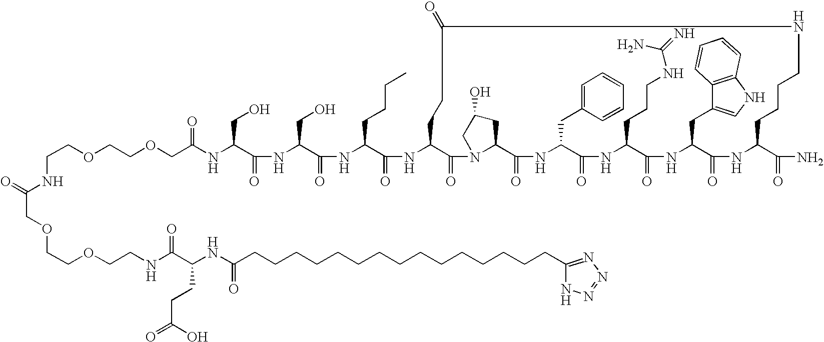

LCMS (system 2): Rt=11.40 min; ((m+2)/2)=937

### Example 18

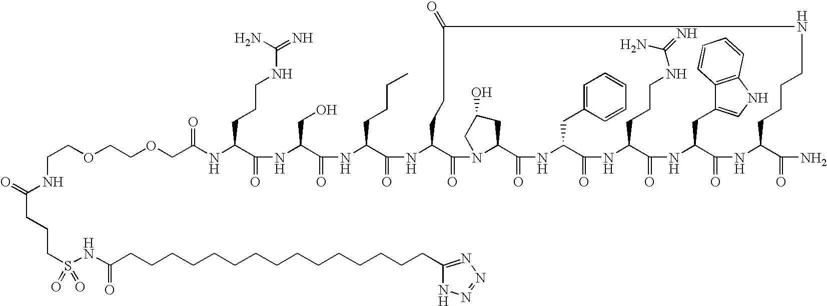

### Example 19

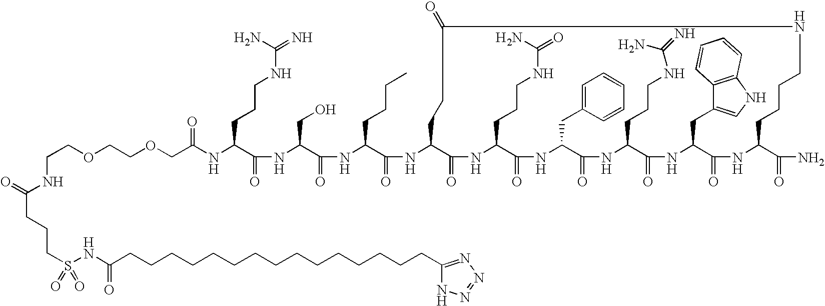

### Example 20

### {2-[2-(15-(Carboxy)pentadecanoylamino)ethoxy]ethoxy}acetyl-Gly-Ser-Gln-His-Dap-Nle-c[Glu-Hyp-D-Phe-Arg-Trp-Lys]-NH₂

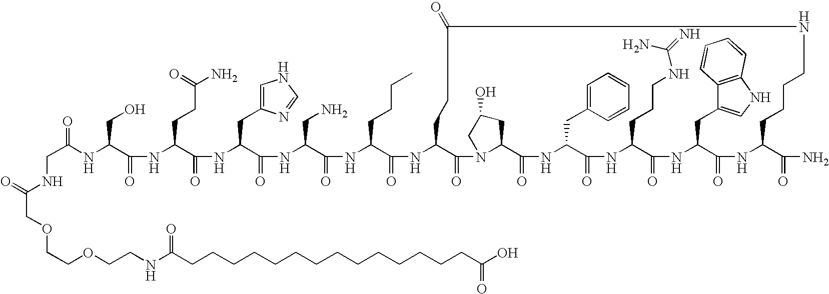

LCMS (system 2): Rt=10.06 min; ((m+2)/2)=941

### Example 21

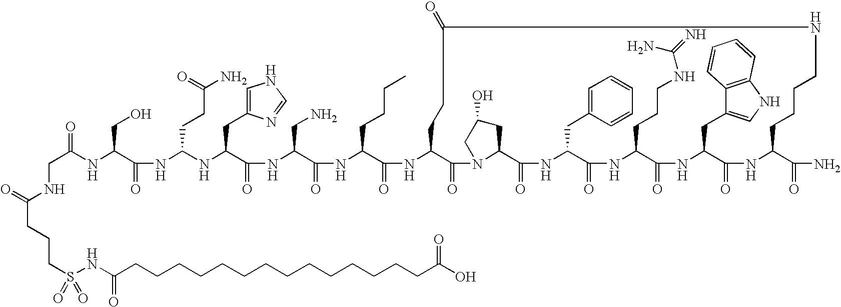

The building block 16-(3-carboxy-propane-1-sulfonylamino)-16-oxo-hexadecanoic acid tert-butyl ester is a suitable starting point for the preparation of this compound. The synthesis of the building block is outlined below.

### Example 22

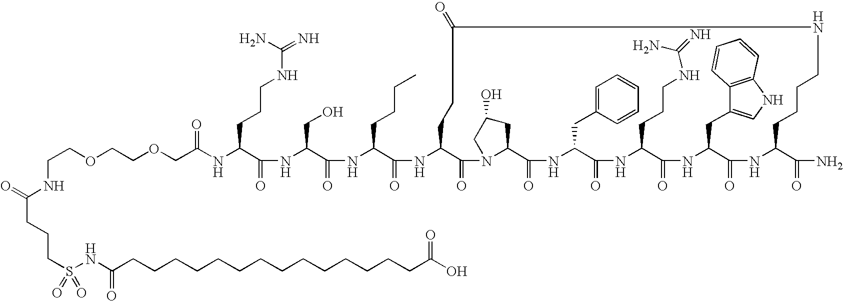

### Example 23

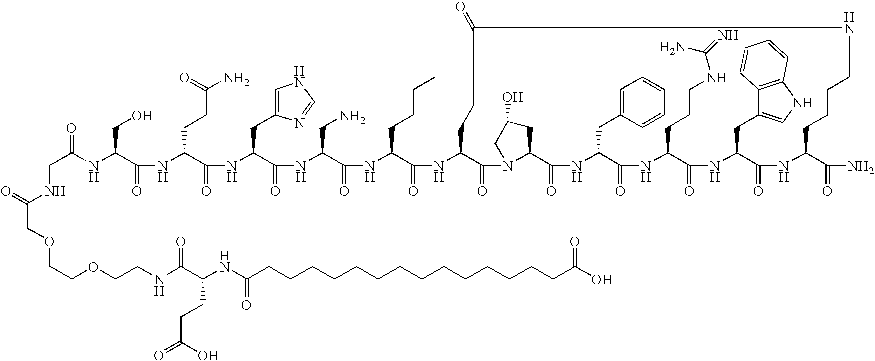

### Example 24

### 15-Carboxypentadecanoyl-Gly-Ser-Ser-Tyr-Thr-Nle-c[Glu-Hyp-D-Phe-Arg-Trp-Lys]-NH₂

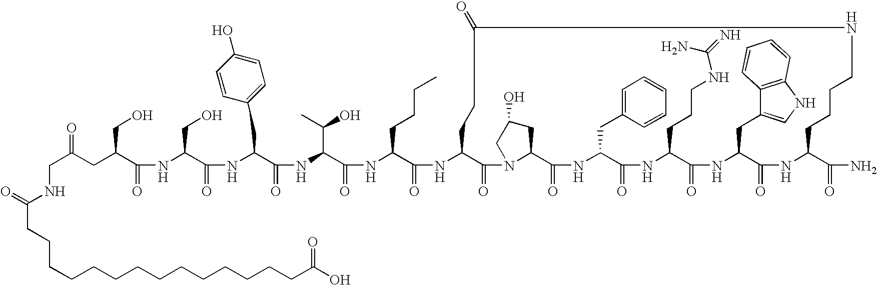

LCMS (system 2): Rt=11.4 min; ((m+2)/2)=869

### Example 25

### {2-[2-(15-Carboxypentadecanoylamino)ethoxy]ethoxy}acetyl-Ser-Tyr-Hyp-Nle-c[Glu-Hyp-D-Phe-Arg-Trp-Lys]-NH₂

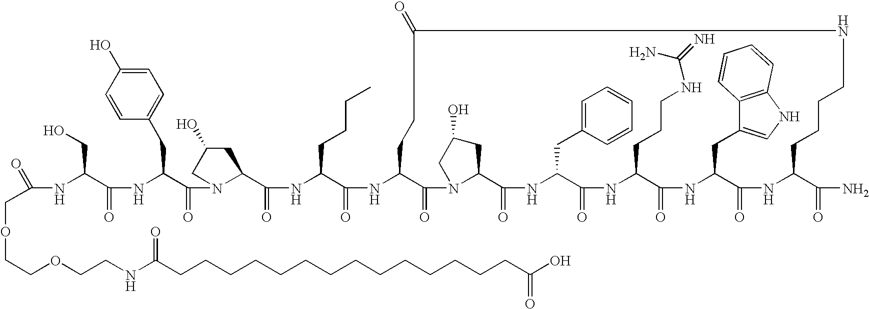

LCMS (system 2): Rt=11.77 min; ((m+2)/2)=875

### Example 26

### {2-[2-(15-Carboxypentadecanoylamino)ethoxy]ethoxy}acetyl-Asn-Asn-Pro-Nle-c[Glu-Hyp-D-Phe-Arg-Trp-Lys]-NH₂

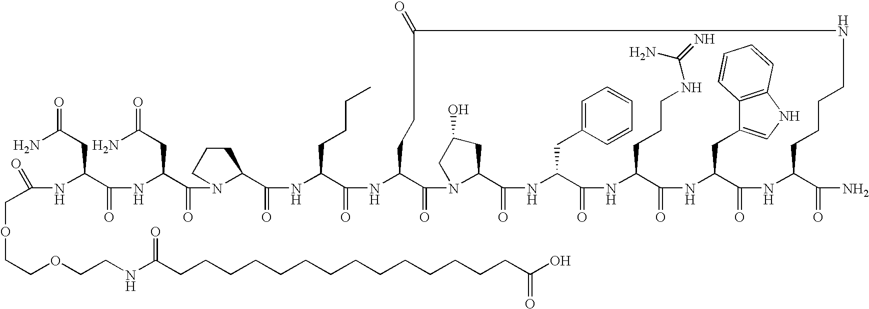

LCMS (system 2): Rt=11.71 min; ((m+2)/2)=857

### Example 27

### (2-{2-[(R)-4-Carboxy-2-(16-(tetrazol-5-yl)hexadecanoylamino)butanoylamino]ethoxy}ethoxy)-acetyl-GIy-Ser-Gln-His-Dap-Nle-c[Glu-Hyp-D-Phe-Arq-Trp-Lysl-NH₂

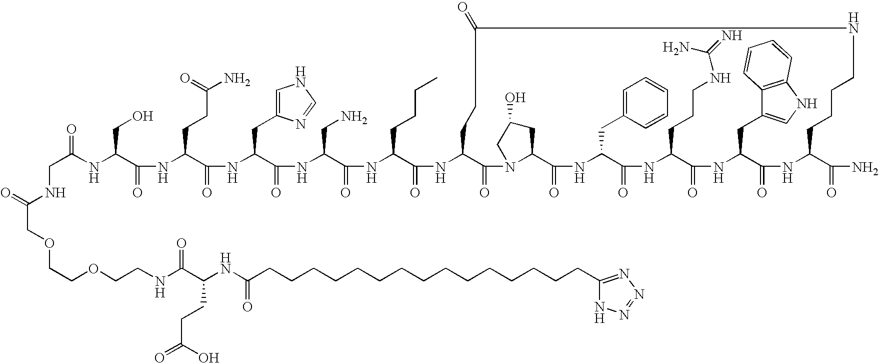

LCMS (system 2): Rt=9.79 min; ((m+2)/2)=1025

### Example 28

### {2-[2-(2-{2-[2-(16-(Tetrazol-5-yl)hexadecanoylamino)ethoxy]ethoxy}acetylamino)ethoxy]ethoxy}acetyl-Ser-Gln-His-Dap-Nle-c[Glu-Hyp-D-Phe-Arg-Trp-Lys]-NH₂

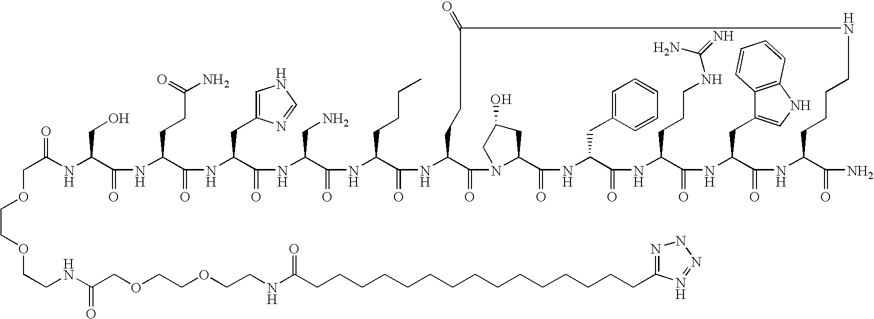

LCMS (system 2): Rt=10.09 min; ((m+2)/2)=1004

### Example 29

### (2-{2-[4-(16-(Tetrazol-5-yl)hexadecanoylsulfamoyl)butanoylamino]ethoxy}ethoxy)acetyl-Arg-Dap-Nle-c[Glu-Hyp-D-Phe-Arg-Trp-Lys]-NH₂

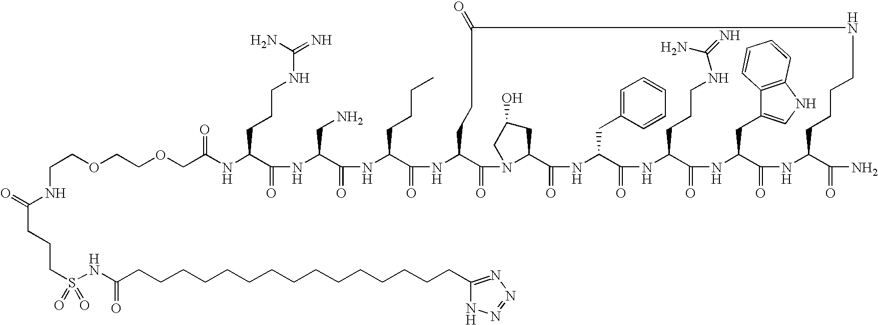

LCMS (system 2): Rt=9.16 min; ((m+2)/2)=908

### Example 30

### {2-[2-(16-(Tetrazol-5-yl)hexadecanoylamino)ethoxy]ethoxy}acetyl-Gly-Ser-Gln-Dap-Ser-Nle-c[Glu-Hyp-D-Phe-Arg-Trp-Lys]-NH₂

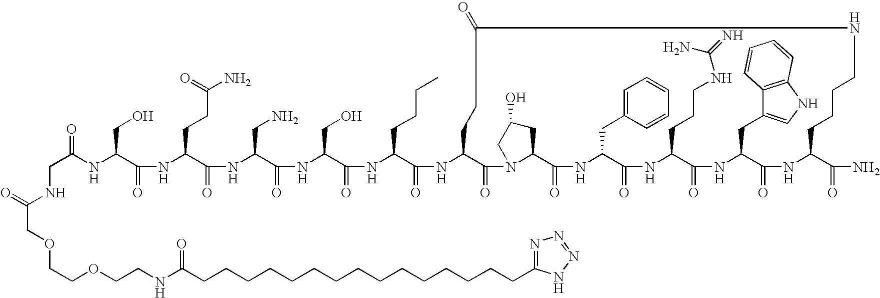

LCMS (system 3): Rt=2.88 min; ((m+2)/2)=936

### Example 31

### (2-{2-[4-(16-(Tetrazol-5-yl)hexadecanoylsulfamoyl)butanoylamino]ethoxy}ethoxy)acetyl-His-Dap-Nle-c[Glu-Hyp-D-Phe-Arg-Trp-Lys]-NH₂

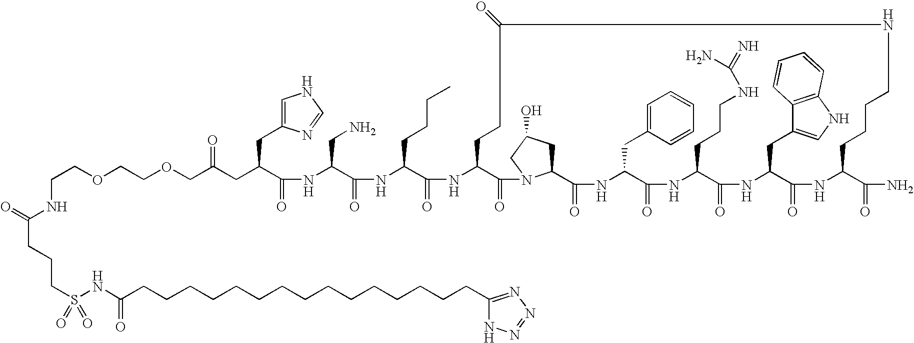

LCMS (system 3): Rt=3.25 min; ((m+2)/2)=899

### Example 32

### {2-[2-(2-{2-[2-(16-(Tetrazol-5-yl)hexadecanoylamino)ethoxy]ethoxy}acetylamino)ethoxy]-ethoxy}acetyl-Dap-Nle-c[Glu-Hyp-D-Phe-Arg-Trp-Lys]-NH₂

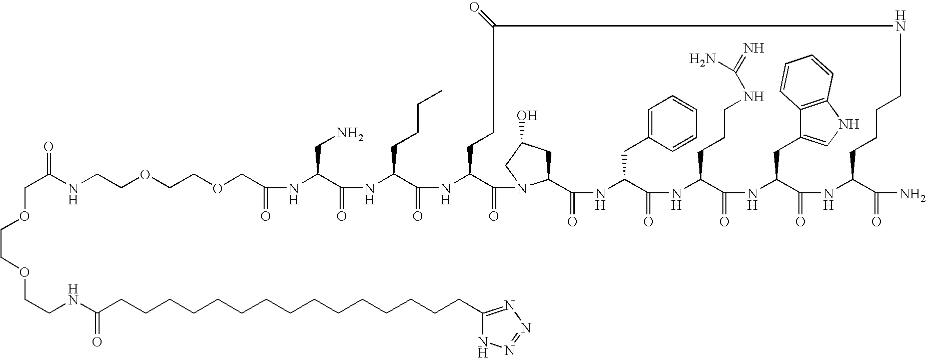

LCMS (system 3): Rt=3.36 min; ((m+2)/2)=828

### Example 33

### (2-{2-[4-(16-(Tetrazol-5-yl)hexadecanoylsulfamoyl)butanoylamino]ethoxy}ethoxy)acetyl-Gly-Ser-Gln-His-Dap-Nle-c[Glu-Hyp-D-Phe-Arg-Trp-Lys]-NH₂

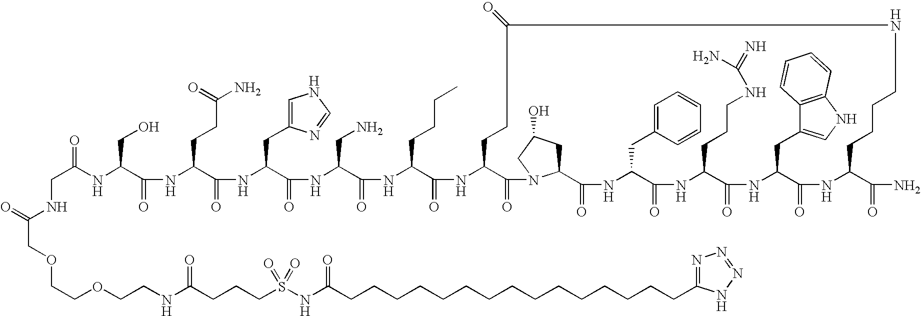

LCMS (system 3): Rt=3.24 min; ((m+2)/2)=1035

### Example 34

### 4-(16-(Tetrazol-5-yl)hexadecanoylsulfamoyl)butanoyl-Gly-Ser-Gln-His-Dap-Nle-c[Glu-Hyp-D-Phe-Arg-Trp-Lys]-NH₂

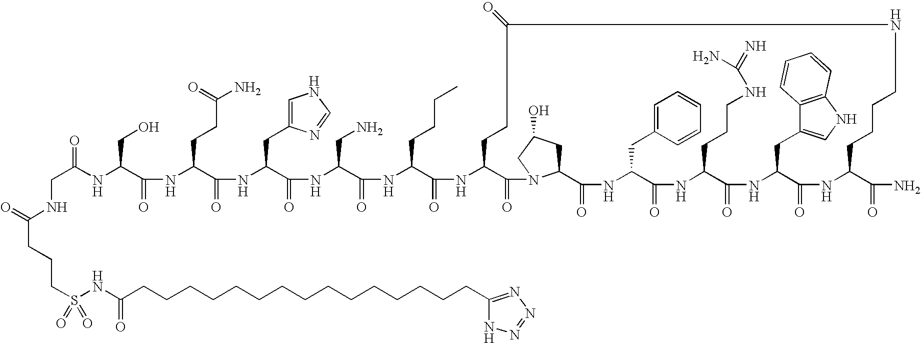

LCMS (system 3): Rt=3.42 min; ((m+2)/2)=963

### Example 35

### {2-[2-(16-(Tetrazol-5-yl)hexadecanoylamino)ethoxy]ethoxy}acetyl-Gly-Ser-D-Ser-His-His-Nle-c[Glu-Hyp-D-Phe-Arg-Trp-Lys]-OH

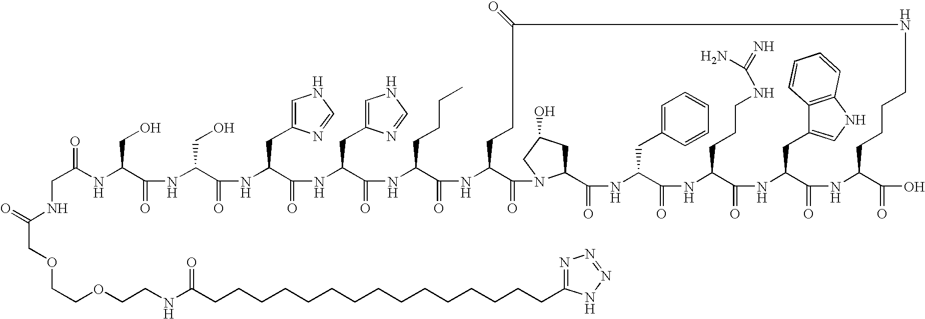

### Example 36

### (2-[2-{(2-[2-{16-(Tetrazol-5-yl)hexadecanoylamino}ethoxy]ethoxy)acetylamino}ethoxy]-ethoxy)acetyl-Gly-Ser-Gln-His-Dap-Nle-c[Glu-Hyp-D-Phe-Arg-Trp-Lys]-NH₂

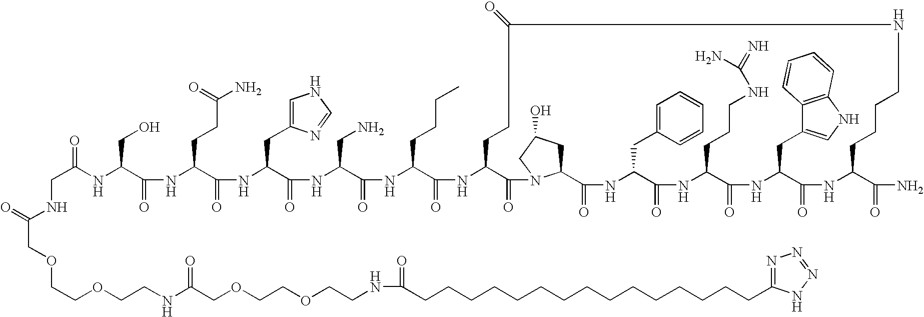

LCMS (system 3): Rt=3.34 min; ((m+3)/3)=689

### Example 37

### (2-[2-{(2-[2-{(2-[2-{(2-[2-{16-(Tetrazol-5-yl)hexadecanoylamino}ethoxy]ethoxy)acetylamino}-ethoxy]ethoxy)acetylamino}ethoxy]ethoxy)acetylamino}ethoxy]ethoxy)acetyl-Gly-Ser-Gln-His-Dap-Nle-c[Glu-Hyp-D-Phe-Arg-Trp-Lys]-NH₂

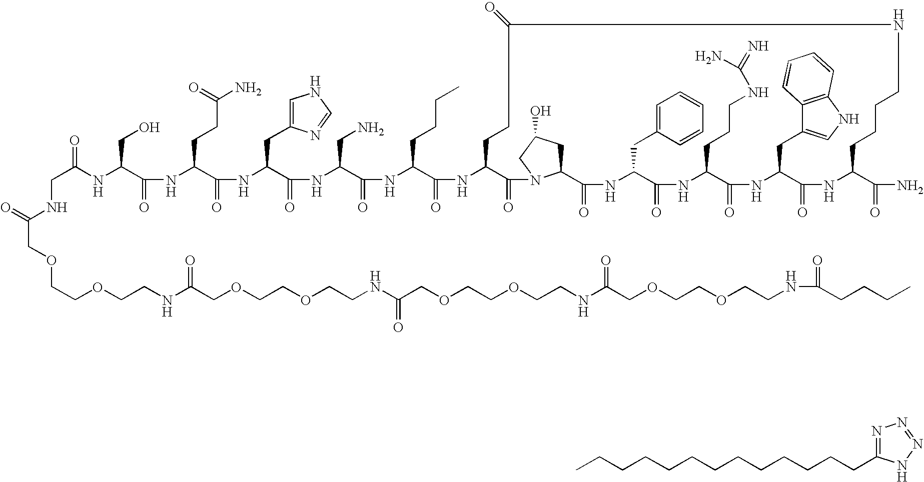

LCMS (system 3): Rt=3.13 min; ((m+3)/3)=786

### Example 38

### {2-[2-(16-(Tetrazol-5-yl)hexadecanoylamino)ethoxy]ethoxy}acetyl-Gly-Ser-Gln-His-His-Nle-c[Glu-Hyp-D-Phe-Arg-Trp-Lys]-NH₂

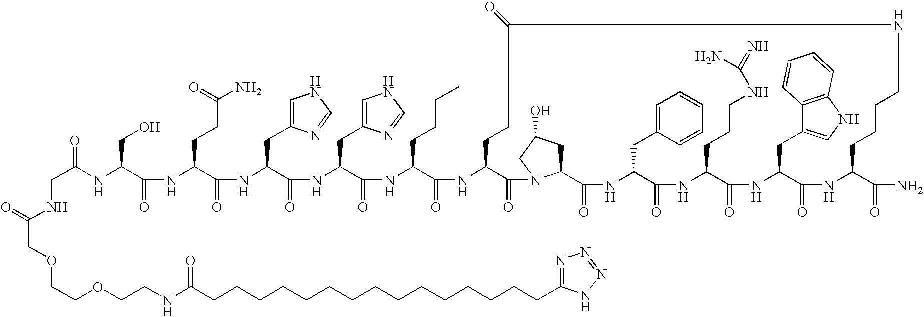

LCMS (system 3): Rt=3.30 min; ((m+2)/2)=986

### Example 39

### {2-[2-(16-(Tetrazol-5-yl)hexadecanoylamino)ethoxy]ethoxy}acetyl-Gly-Ser-Gln-His-Ser-Nle-c[Glu-Hyp-D-Phe-Arg-Trp-Lys]-NH₂

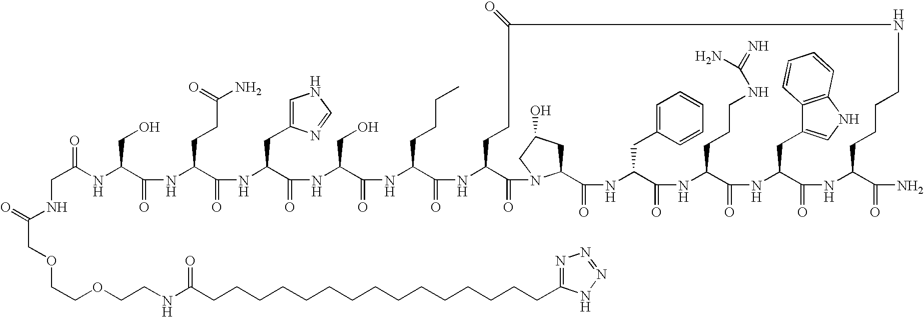

LCMS (system 3): Rt=3.28 min; ((m+2)/2)=961

### Example 40

### {2-[2-(16-(Tetrazol-5-yl)hexadecanoylamino)ethoxy]ethoxy}acetyl-Gly-Ser-Gln-His-Nle-c[Glu-Hyp-D-Phe-Arg-Trp-Lys]-NH₂

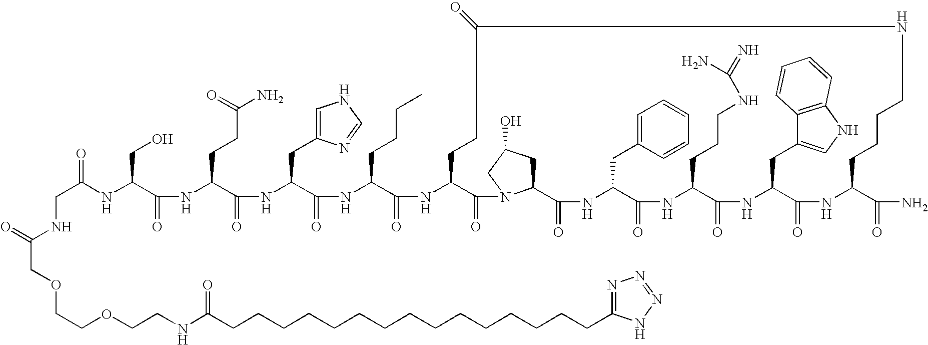

LCMS (system 3): Rt=3.47 min; ((m+3)/3)=612

### Example 41

### 4-(15-Carboxypentadecanoylsulfamoyl)butanoyl-Gly-Ser-Gln-His-Dap-Nle-c[Glu-Hyp-D-Phe-Arg-Trp-Lys]-NH₂

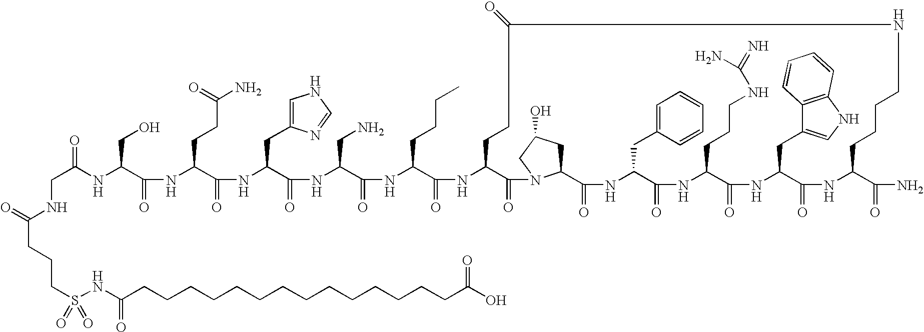

LCMS (system 3): Rt=3.42 min; ((m+2)/2)=943

### Example 42

### (2-{2-[(S)-4-Carboxy-4-(17-carboxyheptadecanoylamino)butanoylamino]ethoxy}ethoxy)-acetyl-Gly-Ser-Gln-His-Dap-Nle-c[Glu-Hyp-D-Phe-Arg-Trp-Lys]-NH₂

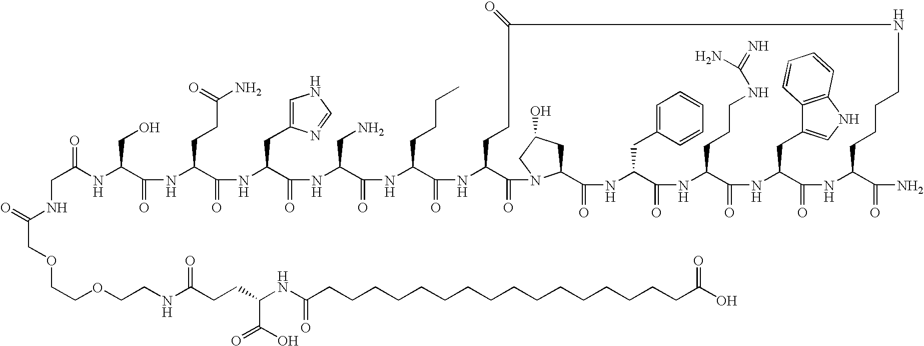

MALDI-MS: m/z=2040.1

### Example 43

### [2-(2-{(S)-4-Carboxy-4-[2-(17-carboxyheptadecanoylamino)acetylamino]butanoylamino}-ethoxy)ethoxy]acetyl-Gly-Ser-Gln-His-Dap-Nle-c[Glu-Hyp-D-Phe-Arg-Trp-Lys]-NH₂

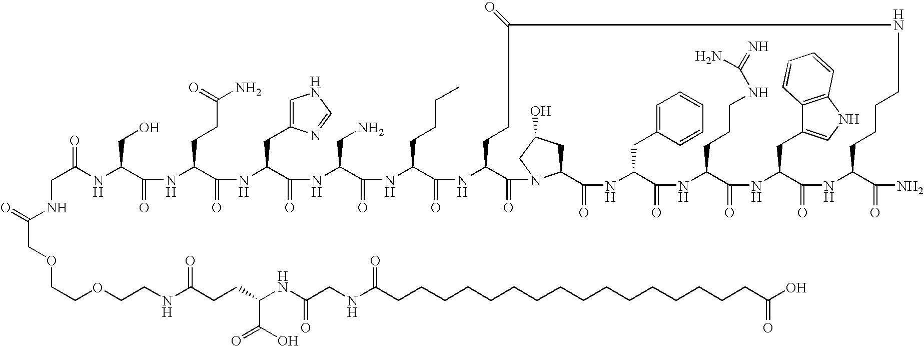

MALDI-MS: m/z=2098.21

### Example 44

### (2-{2-[(S)-3-Carboxy-3-(17-carboxyheptadecanoylamino)propanoylamino]ethoxy}ethoxy)-acetyl-Gly-Ser-Gln-His-Dap-Nle-c[Glu-Hyp-D-Phe-Arg-Trp-Lys]-NH₂

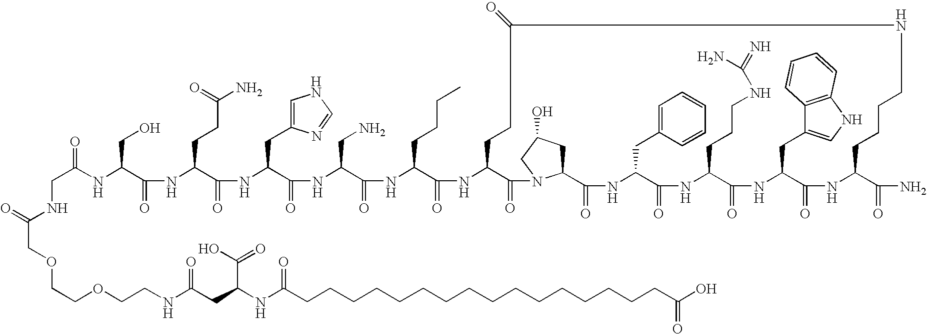

MALDI-MS: m/z=2025.84

### Example 45

### {2-[2-(16-(Tetrazol-5-yl)hexadecanoylamino)ethoxy]ethoxy}acetyl-Gly-Ser-Gln-His-Thr-Nle-c[Glu-Hyp-D-Phe-Arg-Trp-Lys]-NH₂

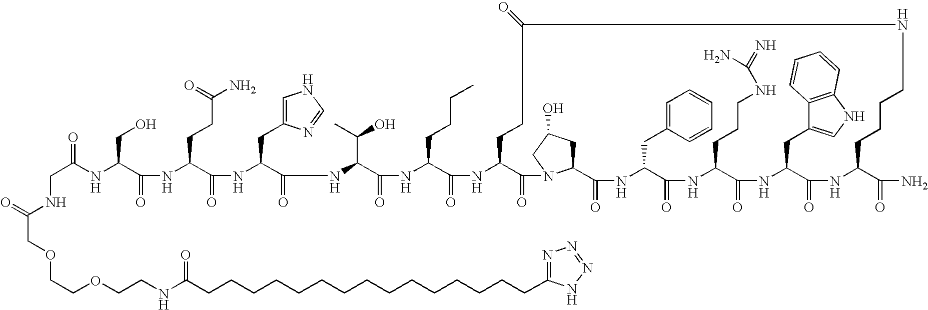

MALDI-MS: m/z=1931.83

### Example 46

{2-[2-(16-(Tetrazol-5-yl)hexadecanoylamino)ethoxy]ethoxy}acetyl-Gly-Ser-Gln-His-Dab-Nle-c[Glu-Hyp-D-Phe-Arg-Trp-Lys]-NH₂

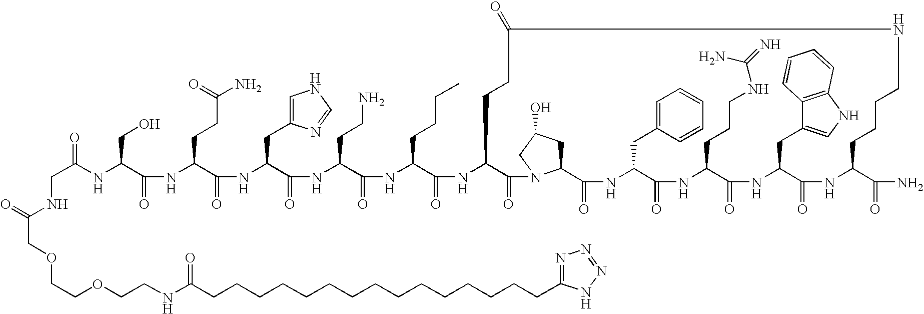

MALDI-MS: m/z=1934.06

### Example 47

### {2-[2-(16-(Tetrazol-5-yl)hexadecanoylamino)ethoxy]ethoxy}acetyl-Gly-Ser-Gln-His-homoSer-Nle-c[Glu-Hyp-D-Phe-Arg-Trp-Lys]-NH₂

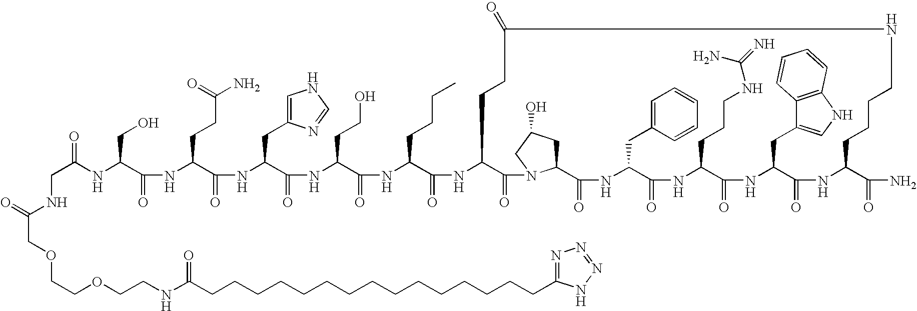

MALDI-MS: m/z=1935.15

### Example 48

### {2-[2-(16-(Tetrazol-5-yl)hexadecanoylamino)ethoxy]ethoxy}acetyl-Gly-Ser-Gln-His-Orn-Nle-c[Glu-Hyp-D-Phe-Arg-Trp-Lys]-NH₂

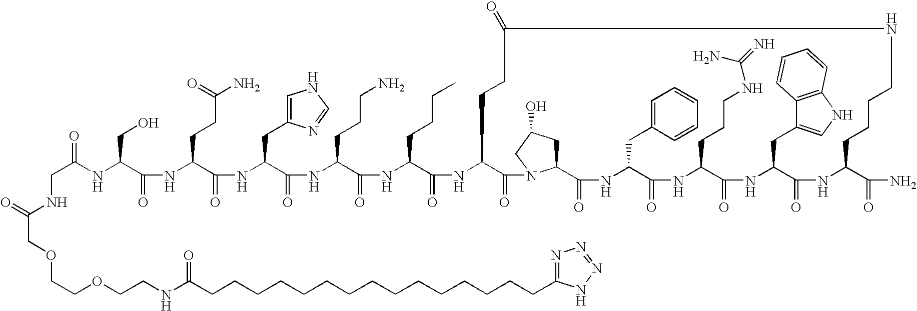

### Example 49

### {2-[2-(16-(Tetrazol-5-yl)hexadecanoylamino)ethoxy]ethoxy}acetyl-Gly-Ser-Gln-His-Lys-Nle-c[Glu-Hyp-D-Phe-Arg-Trp-Lys]-NH₂

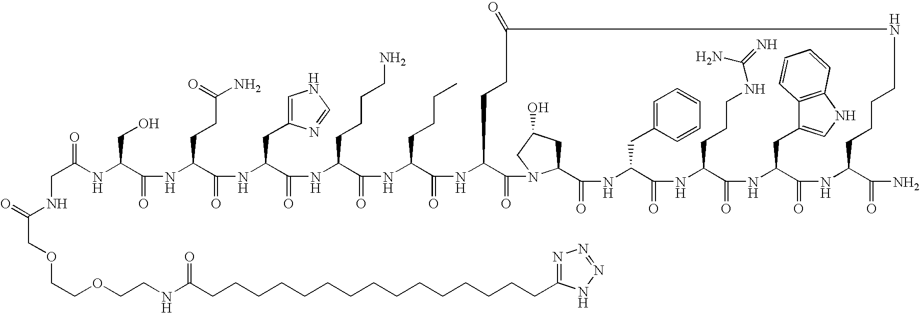

### Example 50

### {2-[2-(16-(Tetrazol-5-yl)hexadecanoylamino)ethoxy]ethoxy}acetyl-Gly-Ser-Gln-His-Arg-Nle-c[Glu-Hyp-D-Phe-Arg-Trp-Lys]-NH₂

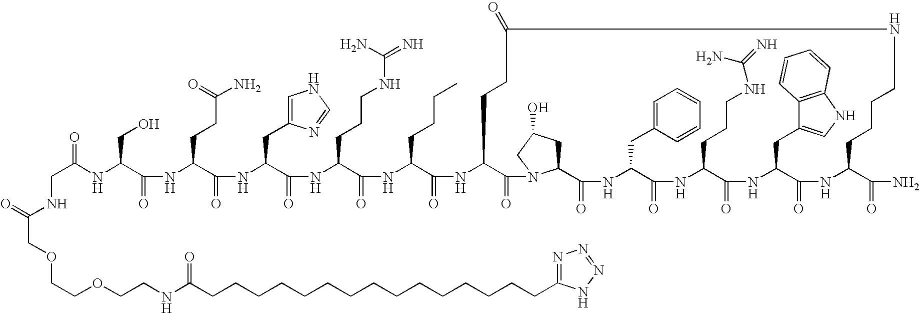

### Example 51

### {2-[2-(16-(Tetrazol-5-yl)hexadecanoylamino)ethoxy]ethoxy}acetyl-Gly-Ser-Gln-His-2-PyAla-Nle-c[Glu-Hyp-D-Phe-Arg-Trp-Lys]-NH₂

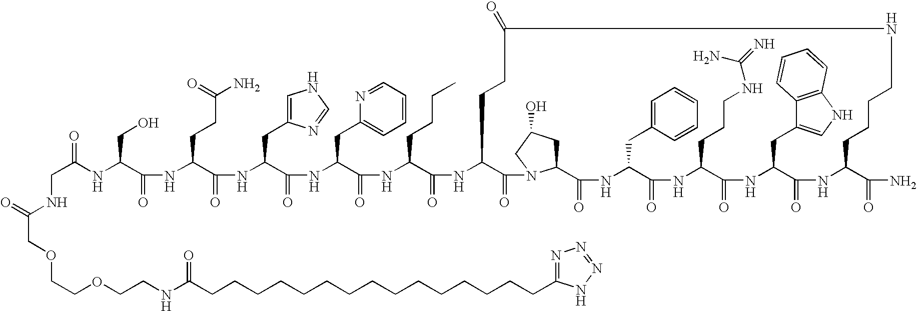

### Example 52

### {2-[2-(16-(Tetrazol-5-yl)hexadecanoylamino)ethoxy]ethoxy}acetyl-Gly-Ser-Gln-His-4-PyAla-Nle-c[Glu-Hyp-D-Phe-Arg-Trp-Lys]-NH₂

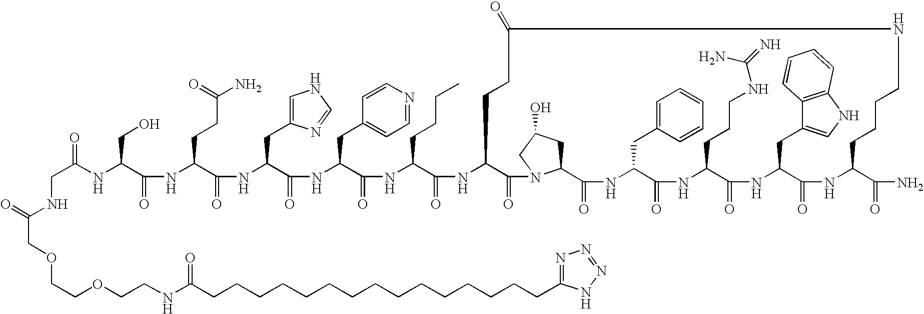

### Preparation of 16-(tetrazol-5-yl)hexadecanoic Acid

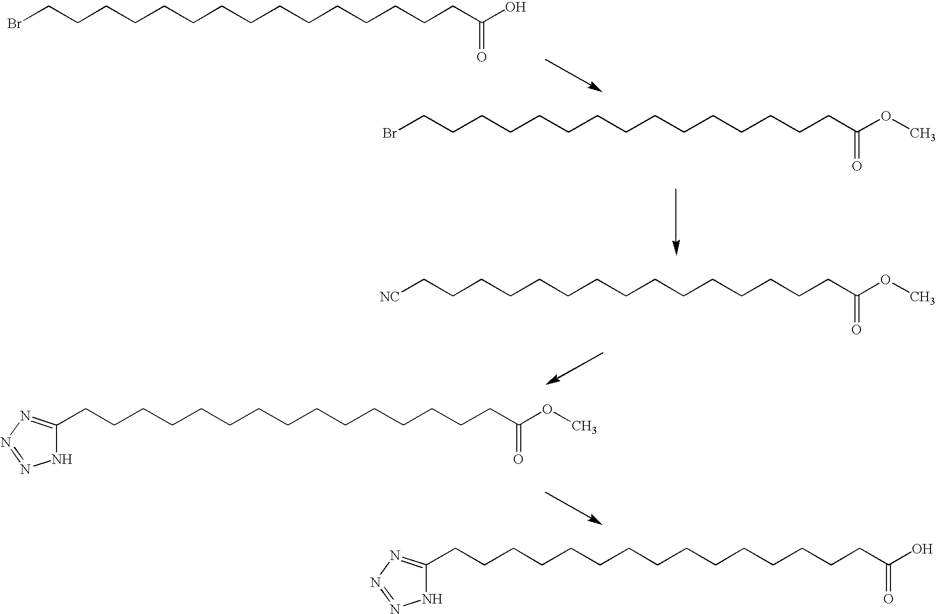

16-Bromohexadecanoic acid (26.83 g, 80 mmol) was suspended in a mixture of methanol (160 ml) and toluene (30 ml). Polymer-bound arenesulfonic acid (1.5 g; macroporous polystyrene beads; “Amberlyst 15”; Fluka 06423) and trimethylorthoformate (17.5 ml, 160 mmol) were added and the mixture was refluxed for 6 h at 90° C. oil bath temperature. The reaction mixture was left to stand overnight at room temperature and then filtered. The resulting filtrate was concentrated under reduced pressure to give crude 16-bromohexadecanoic acid methylester as a brownish liquid.

To the crude methyl ester (80 mmol), NMP (140 ml) and sodium cyanide (9.41 g, 192 mmol) were added. The resulting suspension was stirred at 155° C. for 2 h. After being cooled to room temperature, the resulting dark brown suspension was treated with water (550 ml). Concentrated 37% aqueous HCl (5 ml, approx. 60 mmol, caution, can give deadly HCN gas!) and ice were added to give a suspension of pH 9. The suspension was left to stand for 40 min and then filtered. The resulting filter cake was washed with water (2×125 ml) and dried for 20 h on tissue paper to give a brownish solid mainly consisting of the desired nitrile, but still containing the corresponding alkyl bromide (approx. 20% by ¹H NMR in deutero-chloroform). For repeating the reaction, the residue was mixed with freshly powdered sodium cyanide (6.27 g, 128 mmol) and NMP (100 ml). The resulting dark brown suspension was stirred at 110° C. oil bath temperature for 5 h and then left to stand overnight at room temperature. The mixture was treated with a mixture of water (400 ml) and concentrated 37% aqueous HCl (2.5 ml, approx. 30 mmol, caution, can give deadly HCN gas!), resulting in a suspension of pH 11. Ice was added and the suspension was left to stand for 45 min and then filtered. The resulting filter cake was washed with water (2×125 ml) and dried overnight on tissue paper to give an off-white, pasty residue. According to LCMS and ¹H NMR, this product was mainly the desired 16-cyanohexadecanoic acid methyl ester, along with minor amounts of 16-cyanohexadecanoic acid, water and NMP.

The crude nitrile, freshly powdered sodium azide (20.80 g, 320 mmol) and triethylamine hydrochloride (22.19 g, 160 mmol) were suspended in NMP (200 ml) and stirred at 150° C. oil bath temperature for 18 h. The reaction mixture was left to cool down to room temperature and then poured into a beaker. Water (500 ml) and 37% aqueous HCl (42 ml, approx. 500 mmol) were added. The resulting suspension was stirred, left to stand for 40 min and then filtered. The resulting filter cake was washed with water (250 ml) and dried on the filter for three days to give an off-white pasty residue.

This product was suspended in a mixture of MeOH (180 ml) and aqueous NaOH (11.2 g, 280 mmol, dissolved in 50 ml water). The mixture was stirred at 85° C. oil bath temperature for 3½ h. The oil bath was removed. To the warm solution, water (50 ml) was added. The resulting dim liquid was poured into a beaker and stirred with a mixture of water (400 ml) and 37% aqueous HCl (30 ml, approx. 360 mmol). After addition of ice, the resulting suspension (approx. 800 ml) was left to stand for 50 min and then filtered. The resulting filter cake was washed with water (500 ml) to give a white wet solid.

This product (still wet) was recrystallized from MeCN (550 ml, crystallization overnight). The resulting precipitate was collected by filtration, washed with MeCN (2×100 ml) and petroleum ether (100 ml) and dried on tissue paper for 24 h to give the title compound as a yellowish solid. The resulting filtrate was filtered again and the resulting solid was washed with MeCN (2×100 ml) and dried on tissue paper for 23 h to give the title compound as a brownish solid. 19.71 g (76% yield) of 16-(tetrazol-5-yl)hexadecanoic acid was obtained.

¹H NMR (DMSO-d6) δ=1.23 (m, 22H), 1.47 (m, 2H), 1.67 (m, 2H), 2.18 (t, J=7 Hz, 2H).

### Preparation of 4-(16-tetrazol-5-yl-hexadecanoylsulfamoyl)butyric acid

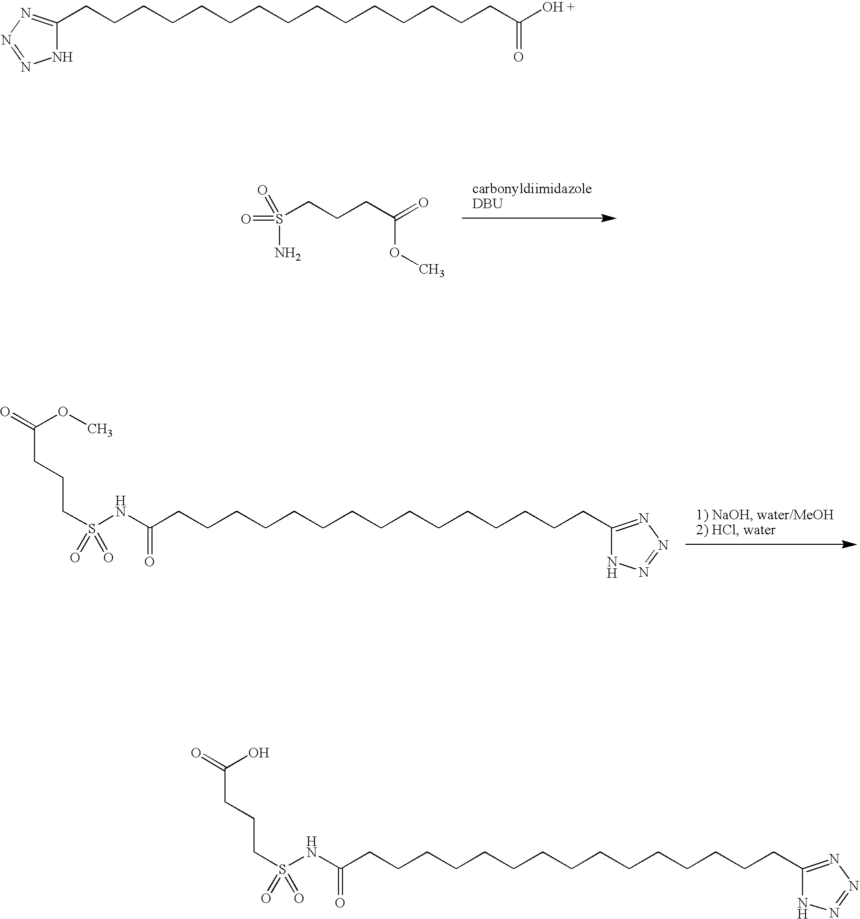

16-(Tetrazol-5-yl)hexadecanoic acid (6.49 g, 20.0 mmol) and carbonyldiimidazole (3.34 g, 20.6 mmol) were mixed. DMF (110 ml) was added and the resulting milky mixture was stirred for 2 h. Then, a solution of (4-sulfamoyl)butyric acid methyl ester (3.62 g, 20.0 mmol) in DMF (20 ml) was added, followed by addition of DBU (6.57 ml, 44.0 mmol). The resulting solution was stirred for 18 h and then poured into 0.1 M aqueous HCl (870 ml) to give a white precipitate. Residual material was washed from the reaction flask into the acidic suspension with MeOH (5 ml). The resulting suspension of pH 4-5 was left to stand for 2½ h and then filtered. The filter cake was washed with 0.01 M aqueous HCl (170 ml) and water (280 ml) to give an off-white wet solid. This product (still wet) was recrystallized from MeCN (300 ml, crystallization overnight). The resulting precipitate was collected by filtration, washed with MeCN (80 ml) and dried on tissue paper to give 5.95 g (61% yield) of 4-(16-tetrazol-5-yl-hexadecanoylsulfamoyl)butyric acid methyl ester as an off-white solid.

¹H NMR (DMSO-d6) δ=1.23 (m, 22H), 1.49 (m, 2H), 1.67 (m, 2H), 1.88 (m, 2H), 2.25 (t, J=7 Hz, 2H), 2.48 (t, J=7 Hz, 2H), 2.85 (t, J=7 Hz, 2H), 3.39 (m, 2H), 3.59 (s, 3H).

The methyl ester (5.95 g, 12.2 mmol) was suspended in MeOH (50 ml). 1 M aqueous NaOH (43 ml, 43 mmol) was added and the resulting solution was stirred for 19 h. The solution was carefully acidified with 0.5 M aqueous HCl (100 ml, 50 mmol). Water (50 ml) was added. The resulting white suspension was left to stand for 45 min and then filtered. The filter cake was washed with water (200 ml) and then recrystallized from MeCN (200 ml, oil bath, yellowish solution when hot, crystallization overnight). The resulting precipitate was collected by filtration, washed with MeCN (100 ml) and dried on tissue paper to give the title compound as a white solid. 5.10 g (54% yield over two steps) of 4-(16-tetrazol-5-yl-hexadecanoylsulfamoyl)butyric acid was obtained.

¹H NMR (DMSO-d6) δ=1.23 (m, 20H), 1.49 (m, 2H), 1.67 (m, 2H), 1.85 (m, 2H), 2.25 (t, J=7 Hz, 2H), 2.38 (t, J=7 Hz, 2H), 2.85 (t, J=7 Hz, 2H), 3.38 (m, partially overlapping with water peak at 3.35 ppm), 12.23 (broad s, 1H).

### Preparation of Hexadecanedioic Acid mono-tert-butyl Ester

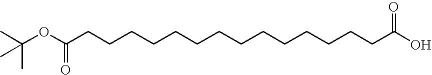

This compound was prepared from hexadecanedioic acid and dimethylformamide-di-tert-butyl acetal according to the general procedure reported in the literature: U. Widmer, Synthesis 1983, 135.

### Preparation of 16-(3-carboxy-propane-1-sulfonylamino)-16-oxo-hexadecanoic acid tert-butyl ester

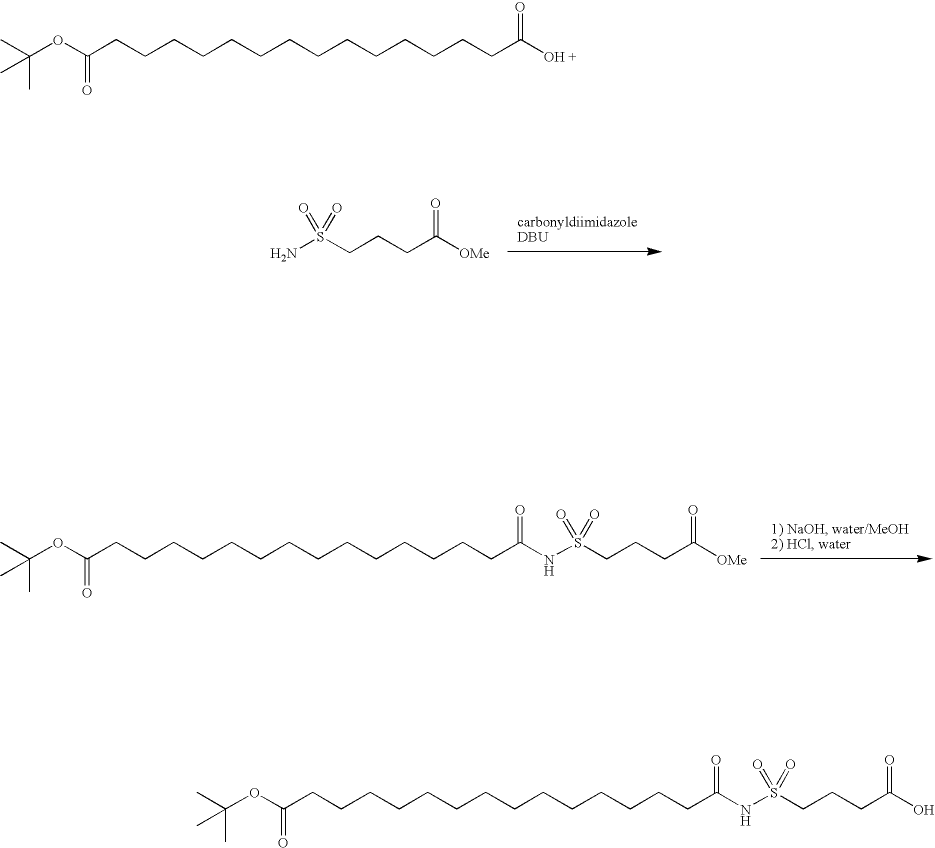

Hexadecanedioic acid mono-tert-butyl ester (5.14 g, 15.0 mmol) was dissolved in DCM (30 ml) and MeCN (30 ml). Carbonyldiimidazole (2.51 g, 15.45 mmol) was added and the mixture was stirred for 2 h. A solution of (4-sulfamoyl)butyric acid methyl ester (2.72 g, 15.0 mmol) in DCM (30 ml) was added, followed by addition of DBU (2.69 ml, 18 mmol). The mixture was stirred overnight and then concentrated under reduced pressure. The resulting residue was treated with 0.2 M aqueous citrate buffer pH 4.5 (preparation of the buffer: 0.2 mol of citric acid and 0.35 mol of NaOH dissolved in one liter of water). After 20 min, the resulting precipitate was collected by filtration and washed with water (150 ml).

This product was dissolved in MeOH (70 ml) and THF (20 ml). 1 M aqueous NaOH (13 ml, 13 mmol) was slowly added and the mixture was stirred. After 40 min, a new portion of 1M aqueous NaOH (14.3 ml, 14.3 mmol) was slowly added. The mixture was stirred overnight and then poured into a mixture of water (150 ml) and 0.2 M aqueous citrate buffer pH 4.5 (150 ml). After 1 h, the resulting precipitate was collected by filtration, washed with water (100 ml) and dried to give the crude title compound. Recrystallization from acetone (300 ml) afforded 2.44 g (33% yield) of 16-(3-carboxy-propane-1-sulfonylamino)-16-oxo-hexadecanoic acid tert-butyl ester.

¹H NMR (DMSO-d6) δ=1.23 (m, 20H), 1.39 (s, 9H), 1.48 (m, 4H), 1.84 (m, 2H), 2.16 (t, J=7 Hz, 2H), 2.24 (t, J=7 Hz, 2H), 2.38 (t, J=7 Hz, 2H), 3.37 (m, partially overlapping with water peak at 3.33 ppm).

### Pharmacological Methods

Assay (I)—Experimental Protocol for Efficacy Testing on Appetite with MC4 Analogues, Using an ad libitum Fed Rat Model.

TAC:SPRD @mol rats or Wistar rats from M&B Breeding and Research Centre A/S, Denmark are used for the experiments. The rats have a body weight 200-250 g at the start of experiment. The rats arrive at least 10-14 days before start of experiment with a body weight of 180-200 g. Each dose of compound is tested in a group of 8 rats. A vehicle group of 8 rats is included in each set of testing.

When the animals arrive they are housed individually in a reversed light/dark phase (lights off 7:30 am, lights on 7:30 pm), meaning that lights are off during daytime and on during nighttime. Since rats normally initiate food intake when light is removed, and eat the major part of their daily food intake during the night, this set up results in an alteration of the initiation time for food intake to 7:30 am, when lights are switched off. During the acclimatization period of 10-14 days, the rats have free access to food and water. During this period the animals are handled at least 3 times. The experiment is conducted in the rats' home cages. Immediately before dosing the rats are randomised to the various treatment groups (n=8) by body weight. They are dosed according to body weight at between 7:00 am and 7:45 am, with a 1-3 mg/kg solution administered intraperitoneally (ip), orally (po) or subcutaneously (sc). The time of dosing is recorded for each group. After dosing, the rats are returned to their home cages, where they then have access to food and water. The food consumption is recorded individually every hour for 7 hours, and then after 24 h and sometimes 48 h. At the end of the experimental session, the animals are euthanised.

The individual data are recorded in Microsoft excel sheets. Outliers are excluded after applying the Grubbs statistical evaluation test for outliers, and the result is presented graphically using the GraphPad Prism program.

Assay (II)—Melanocortin Receptor 3 and 5 (MC3 and MC5) cAMP Functional Assay Using the AlphaScreen™ cAMP Detection Kit

The cAMP assays for MC3 and MC5 receptors are performed on cells (either HEK293 or BHK cells) stably expressing the MC3 and MC5 receptors, respectively. The receptors are cloned from cDNA by PCR and inserted into the pcDNA 3 expression vector. Stable clones are selected using 1 mg/ml G418.

Cells at approx. 80-90% confluence are washed 3× with PBS, lifted from the plates with Versene and diluted in PBS. They are then centrifuged for 2 min at 1300 rpm, and the supernatant removed. The cells are washed twice with stimulation buffer (5 mM HEPES, 0.1% ovalbumin, 0.005% Tween™ 20 and 0.5 mM IBMX, pH 7.4), and then resuspended in stimulation buffer to a final concentration of 1×10⁶ or 2×10⁶ cells/ml. 25 μl of cell suspension is added to the microtiter plates containing 25 μl of test compound or reference compound (all diluted in stimulation buffer). The plates are incubated for 30 minutes at room temperature (RT) on a plate-shaker set to a low rate of shaking. The reaction is stopped by adding 25 μl of acceptor beads with anti-cAMP, and 2 min later 50 μl of donor beads per well with biotinylated cAMP in a lysis buffer. The plates are then sealed with plastic, shaken for 30 minutes and allowed to stand overnight, after which they are counted in an Alpha™ microplate reader.

EC₅₀ values are calculated by non-linear regression analysis of dose/response curves (6 points minimum) using the Windows™ program GraphPad™ Prism (GraphPad™ Software, USA). All results are expressed in nM.

For measuring antagonistic activity in the MC3 functional cAMP assay, the MC3 receptors are stimulated with 3 nM α-MSH, and inhibited by increasing the amount of potential antagonist. The IC₅₀ value for the antagonist is defined as the concentration that inhibits MC3 stimulation by 50%.

Assay (III)—Melanocortin Receptor 4 (MC4) cAMP Assay

BHK cells expressing the MC4 receptor are stimulated with potential MC4 agonists, and the degree of stimulation of cAMP is measured using the Flash Plate® cAMP assay (NEN™ Life Science Products, cat. No. SMP004).

The MC4 receptor-expressing BHK cells are produced by transfecting the cDNA encoding MC4 receptor into BHK570/KZ10-20-48, and selecting for stable clones expressing the MC4 receptor. The MC4 receptor cDNA, as well as a CHO cell line expressing the MC4 receptor, may be purchased from Euroscreen™. The cells are grown in DMEM, 10% FCS, 1 mg/ml G418, 250 nM MTX and 1% penicillin/streptomycin.

Cells at approx. 80-90% confluence are washed 3× with PBS, lifted from the plates with Versene and diluted in PBS. They are then centrifuged for 2 min at 1300 rpm, and the supernatant removed. The cells are washed twice with stimulation buffer, and resuspended in stimulation buffer to a final concentration of 0.75×10⁶ cells/ml (consumption thereof: 7 ml per 96-well microtiter plate). 50 μl of cell suspension is added to the Flash Plate containing 50 μl of test compound or reference compound (all diluted in H₂O). The mixture is shaken for 5 minutes and then allowed to stand for 25 minutes at RT. The reaction is stopped by addition of 100 μl Detection Mix per well (Detection Mix=11 ml Detection Buffer+100 μl (˜2 μCi) cAMP [¹²⁵I] tracer). The plates are then sealed with plastic, shaken for 30 minutes, and allowed to stand overnight (or for 2 hours) and then counted in the Topcounter (2 min/well). The assay procedure and the buffers are generally as described in the Flash Plate kit-protocol (Flash Plate® cAMP assay (NEN™ Life Science Products, cat. No. SMP004)). However the cAMP standards are diluted in 0.1% HSA and 0.005% Tween™ 20 and not in stimulation buffer.

EC₅₀ values are calculated by non-linear regression analysis of dose/response curves (6 points minimum) using the Windows™ program GraphPad™ Prism (GraphPad Software, USA). All results are expressed in nM.

### Assay (IV)—Melanocortin Receptor 1 (MC1) Binding Assay

The MC1 receptor binding assay is performed on BHK cell membranes stably expressing the MC1 receptor. The assay is performed in a total volume of 250 μl: 25 μl of ¹²⁵NDP-α-MSH (22 pM in final concentration), 25 μl of test compound/control and 200 μl of cell membrane (35 μg/ml). Test compounds are dissolved in DMSO. Radioactively labeled ligand, membranes and test compounds are diluted in buffer: 25 mM HEPES, pH 7.4, 0.1 mM CaCl₂, 1 mM MgSO₄, 1 mM EDTA, 0.1% HSA and 0.005% Tween™ 20. Alternatively, HSA may be substituted with ovalbumin. The samples are incubated at 30° C. for 90 min in Greiner microtiter plates, separated with GF/B filters that are pre-wetted for 60 min in 0.5% PEI, and washed 2-3 times with NaCl (0.9%) before separation of bound from unbound radiolabelled ligand by filtration. After filtration the filters are washed 10 times with ice-cold 0.9% NaCl. The filters are dried at 50° C. for 30 min, sealed, and 30 μl of Microscint 0 (Packard, cat. No. 6013616) is added to each well. The plates are counted in a Topcounter (1 min/well).

The data are analysed by non-linear regression analysis of binding curves, using the Windows™ program GraphPad™ Prism (GraphPad Software, USA).

### Assay (V)—Melanocortin Receptor 4 (MC4) Binding Assay

In vitro ¹²⁵NDP-α-MSH Binding to Recombinant BHK Cells Expressing Human MC4 Receptor (Filtration Assay).

The assay is performed in 5 ml minisorb vials (Sarstedt No. 55.526) or in 96-well filterplates (Millipore MADVN 6550), and using BHK cells expressing the human MC4 receptor (obtained from Professer Wikberg, Uppsala, Sweden). The BHK cell membranes are kept at −80° C. until assay, and the assay is run directly on a dilution of this cell membrane suspension, without further preparation. The suspension is diluted to give maximally 10% specific binding, i.e. to approx. 50-100 fold dilution. The assay is performed in a total volume of 200 μl: 50 μl of cell suspension, 50 μl of ¹²⁵NDP-α-MSH (≈79 pM in final concentration), 50 μl of test compound and 50 μl binding buffer (pH 7) mixed and incubated for 2 h at 25° C. [binding buffer: 25 mM HEPES, pH 7.0, 1 mM CaCl₂, 1 mM MgSO₄, 1 mM EGTA, 0.02% Bacitracin, 0.005% Tween™ 20 and 0.1% HSA or, alternatively, 0.1% ovalbumin (Sigma; catalogue No. A-5503)]. Test compounds are dissolved in DMSO and diluted in binding buffer. Radiolabelled ligand and membranes are diluted in binding buffer. The incubation is stopped by dilution with 5 ml ice-cold 0.9% NaCl, followed by rapid filtration through Whatman GF/C filters pre-treated for 1 hour with 0.5% polyethyleneimine. The filters are washed with 3×5 ml ice-cold NaCl. The radioactivity retained on the filters is counted using a Cobra II auto gamma counter.

The data are analysed by non-linear regression analysis of binding curves, using the Windows™ program GraphPad™ Prism (GraphPad Software, USA).

### Assay (VI)—Evaluation of Energy Expenditure

TAC:SPRD rats or Wistar rats from M&B Breeding and Research Centre A/S, Denmark are used. After at least one week of acclimatization, rats are placed individually in metabolic chambers (Oxymax system, Columbus Instruments, Columbus, Ohio, USA; systems calibrated daily). During the measurements, animals have free access to water, but no food is provided to the chambers. Light:dark cycle is 12 h:12 h, with lights being switched on at 6:00. After the animals have spent approx. 2 hours in the chambers (i.e. when the baseline energy expenditure is reached), test compound or vehicle are administered (po, ip or sc), and recording is continued in order to establish the action time of the test compound. Data for each animal (oxygen consumption, carbon dioxide production and flow rate) are collected every 10-18 min for a total of 22 hours (2 hours of adaptation (baseline) and 20 hours of measurement). Correction for changes in O₂ and CO₂ content in the inflowing air is made in each 10-18 min cycle.

Data are calculated per metabolic weight [(kg body weight)^{0.75}] for oxygen consumption and carbon dioxide production, and per animal for heat. Oxygen consumption (VO₂) is regarded as the major energy expenditure parameter of interest.

### Assay (VII)—Evaluation of Binding to Albumin

Test compounds are tested in a functional assay (Assay III) and a binding assay (Assay V), wherein Assay III contains HSA, and Assay V contains ovalbumin. EC₅₀ values are determined from Assay III, and Ki values from Assay V. The ratio EC₅₀/Ki is then calculated. In the event of no albumin binding the ratio EC₅₀/Ki will be 1 or below. The stronger the binding to albumin, the higher will be the ratio; for albumin-binding test compounds, the ratio EC₅₀/Ki will thus be ≧1, such as ≧10, e.g. ≧100.

### Assay (VIII)—Melanocortin Receptor 3 (MC3) Binding Assay

The MC3 receptor binding assay is performed on BHK cell membranes stably expressing the human MC3 receptor. The human MC3 receptor is cloned by PCR and subcloned into pcDNA3 expression vector. Cells stably expressing the human MC3 receptor are generated by transfecting the expression vector into BHK cells and using G418 to select for MC3 clones. The BHK MC3 clones are cultured in DMEM with glutamax, 10% FCS, 1% pen/strep and 1 mg/ml G418 at 37° C. and 5% CO₂.

The binding is performed on a membrane preparation prepared in the following way: The cells are rinsed with PBS and incubated with Versene for approximately 5 min before harvesting. The cells are flushed with PBS and the cell-suspension is centrifuged for 10 min at 2800×G. The pellet is resuspended in 20 ml buffer (20 mM Tris pH 7.2+5 mM EDTA+1 mg/ml Bacitracin (Sigma B-0125)) and homogenized with a glass-teflon homogenizer, 10 times and low speed. The cell suspension is centrifuged at 4° C., 4100×G for 20 min. Pellet is resuspended in buffer and the membranes are diluted to a protein concentration of 1 mg/ml in buffer, aliquoted and kept at −80° C. until use.

The assay is performed in a volume of 100 μl. Mix in the following order 25 μl test compound, 25 μl ¹²⁵I-NDP-α-MSH (app. 60 000 cpm/well˜0.25 nM in final concentration) and 50 μl membranes (30 μg/well) and incubate in Costar round-bottom wells microtiter plate, (catalogue number 3365). Test-compounds are dissolved in DMSO or H₂O. Radioligand, membranes and test compounds are diluted in buffer; (25 mM HEPES pH 7.4, 1 mM CaCl2, 5 mM MgSO4, 0.1% Ovalbumin (Sigma A-5503), 0.005% Tween-20 and 5% Hydroxypropyl-β-cyclodextrin 97%, (Acros organics, code 297561000). The assay mixture is incubated for 1 h at 20-25° C. Incubation is terminated by filtration on a Packard harvester filtermate 196. Rapid filtration through Packard Unifilter-96 GF/B filters pre-treated for 1 h with 0.5% poly-ethylenimine is carried out. The filters are washed with ice-cold 0.9% NaCl 8-10 times. The plate is air dried at 55° C. for 30 min, and 50 μl Microscint 0 (Packard) is added. The radioactivity retained on the filter is counted using a Packard TopCount.NXT.

Results; IC₅₀ values are calculated by non-linear regression analysis of binding curves (6 points minimum) using the windows program GraphPad Prism, GraphPad software, USA. Ki-values were calculated according to the Cheng-Prusoff equation [Y-C. Cheng and W. H. Prusoff, Biochem. Pharmacol. 22 (1973) pp. 3099-3108].

### Assay (IX)—Melanocortin Receptor 5 (MC5) Binding Assay

The MC5 receptor binding assay is performed on BHK cell membranes stably expressing the human MC3 receptor. The human MC5 receptor is cloned by PCR and subcloned into pcDNA3 expression vector. Cells stably expressing the human MC5 receptor are generated by transfecting the expression vector into BHK cells and using G418 to select for MC5 clones. The BHK MC5 clones are cultured in DMEM with glutamax, 10% FCS, 1% pen/strep and 1 mg/ml G418 at 37° C. and 5% CO₂.

The binding is performed on a membrane preparation prepared in the following way: The cells are rinsed with PBS and incubated with Versene for approximately 5 min before harvesting. The cells are flushed with PBS and the cell suspension is centrifuged for 10 min at 2800×G. The pellet is resuspended in 20 ml buffer (20 mM Tris pH 7.2+5 mM EDTA+1 mg/ml Bacitracin (Sigma B-0125)) and homogenized with a glass-teflon homogenizer, 10 times and low speed. The cell-suspension is centrifuged at 4° C., 4100×G for 20 min. Pellet is resuspended in buffer and the membranes are diluted to a protein concentration of 1 mg/ml in buffer, aliquoted and kept at −80° C. until use.

The assay is performed in a volume of 100 μl. Mix in the following order 25 μl test-compound, 25 μl ¹²⁵I-NDP-α-MSH (app. 60 000 cpm/well˜0.25 nM in final concentration) and 50 μl membranes (30 μg/well) and incubate incubation in Costar round-bottom wells microtiter plate, catalogue number 3365: Test-compounds are dissolved in DMSO or H₂O. Radioligand, membranes and test-compounds are diluted in buffer; (25 mM HEPES pH 7.4, 1 mM CaCl2, 5 mM MgSO4, 0.1% Ovalbumin (Sigma A-5503), 0.005% Tween-20 and 5% Hydroxypropyl-β-cyclodextrin, d(97%, Acros organics, code 297561000). The assay mixture is incubated for 1 h at 20-25° C. Incubation is terminated by filtration on a Packard harvester filtermate 196. Rapid filtration through Packard Unifilter-96 GF/B filters pre-treated for 1 h with 0.5% poly-ethylenimine is carried out. The filters are washed with ice-cold 0.9% NaCl 8-10 times. The plate is air dried at 55° C. for 30 min, and 50 μl Microscint 0 (Packard) is added. The radioactivity retained on the filter is counted using a Packard TopCount.NXT.

Results: IC₅₀ values are calculated by non-linear regression analysis of binding curves (6 points minimum) using the windows program GraphPad Prism, GraphPad software, USA. Ki-values were calculated according to the Cheng-Prusoff equation [Y-C. Cheng and W. H. Prusoff, Biochem. Pharmacol. 22 (1973) pp. 3099-3108].

Assay (X)—Melanocortin Receptor 3 (MC3) cAMP Functional Assay Using the FlashPlate® cAMP Detection Kit

The MC3-containing BHK cells are stimulated with potential MC3 agonists, and the degree of stimulation of cAMP is measured using the FlashPlate® cAMP assay, cat. No SMP004, NEN™ Life Science Products.

BHK/hMC3 clone 5 cells: the cells are produced by transfecting the cDNA encoding MC3 receptor into BHK570, and selecting for stable clones expressing the hMC3 receptor. The cells are grown in DMEM, 10% FCS, 1 mg/ml G418 and 1% pen/strep.

Cells at approx. 80-90% confluence are washed with PBS, lifted from the plates with Versene and diluted in PBS. After centrifugation for 5 min at 1300 rpm the supernatant is removed, and the cells are resuspended in stimulation buffer to a final concentration of 2×10⁶ cells/ml. 50 μl cell suspension is added to the Flashplate containing 50 μl of test-compound or reference compound (all dissolved in DMSO and diluted in 0.1% HSA (Sigma A-1887) and 0.005% Tween 20). The mixture is shaken for 5 minutes and then allowed to stand for 25 minutes at room temperature. The reaction is stopped with 100 μl Detection Mix pro well (Detection Mix=11 ml Detection Buffer+100 μl (˜2 μCi) cAMP [¹²⁵I] Tracer). The plates are then sealed with plastic, shaken for 30 minutes and allowed to stand overnight (or for 2 h), and then counted in the Topcounter, 2 min/well (Note that in general, the assay procedure described in the kit-protocol is followed; however, the cAMP standards are diluted in 0.1% HSA and 0.005% Tween 20, and not in stimulation buffer).

Results: EC₅₀ values are calculated by non-linear regression analysis of dose-response curves (6 points minimum) using the Windows program GraphPad Prism, GraphPad Software, USA. Result are expressed in nm. Emax values are calculated as % of NDP-α-MSH maximal stimulation in the hMC3cAMP assay (maximal NDP-α-MSH stimulation=100%).

1. A pharmaceutical composition comprising a compound according to formula I:
T-A-L-P [I]
wherein
T represents tetrazol-5-yl;
A represents a straight-chain, branched and/or cyclic C₆₋₂₀alkyl, C₆₋₂₀alkenyl or C₆₋₂₀alkynyl which may optionally be substituted with one or more substituents selected from halogen, hydroxy and aryl;
L is a bond or a chemical structure covalently linking A and P; and
P represents a peptide structure comprising at least six α-amino acid residues,
in combination with bariatric surgical intervention,
for treating a patient in need thereof in order to achieve weight loss or prevent weight gain in said patient.

2. The composition according to claim 1, wherein T-A represents 10-(tetrazol-5-yl)decyl, 11-(tetrazol-5-yl)undecyl, 12-(tetrazol-5-yl)dodecyl, 13-(tetrazol-5-yl)tridecyl, 14-(tetrazol-5-yl)tetradecyl, 15-(tetrazol-5-yl)pentadecyl, 16-(tetrazol-5-yl)hexadecyl, 17-(tetrazol-5-yl)heptadecyl; 18-(tetrazol-5-yl)octadecyl or 19-(tetrazol-5-yl)nonadecyl.

3. A pharmaceutical composition comprising a compound according to formula II:
R¹—R²—C(═O)—R³—S¹Z¹-Z²-Z³-Z⁴-Z⁵-Z⁶-c[X¹—X²—X³-Arg-X⁴—X⁵]—R⁴ [II]
wherein
R¹ represents tetrazol-5-yl or carboxy;
R² represents a straight-chain, branched and/or cyclic C₆₋₂₀alkyl, C₆₋₂₀alkenyl or C₆₋₂₀alkynyl which may optionally be substituted with one or more substituents selected from halogen, hydroxy and aryl;
R³ is absent or represents —NH—S(═O)₂—(CH₂)₃₋₅—C(═O)— or a peptide fragment comprising one or two amino acid residues and containing at least one carboxy group;
S¹ is absent or represents a 4-aminobutyric acid residue, Gly, β-Ala, or a glycolether-based structure according to one of the formulas IIIa-IIIh;
—HN—CH₂—CH₂—O—CH₂‘CH₂—O—CH₂—C(═O)— └IIIa┘
—[HN—CH₂—CH₂—O—CH₂—CH₂—O—CH₂—C(═O)]₂— [IIIb]
—└HN—CH₂—CH₂—O—CH₂—CH₂—O—CH₂—C(═O)┘₃₋₅— └IIIc┘
—[HN—CH₂—CH₂—O—CH₂—CH₂—O—CH₂—CH₂—NH—C(═O)—CH₂—CH₂—CH₂—C(═O)]₁₋₃— [IIId]
—[HN—CH₂—CH₂—O—CH₂—CH₂—O—CH₂—CH₂—NH—C(═O)—CH₂—O—CH₂—C(═O)]₁₋₃— [IIIe]
—[HN—CH₂—CH₂—O—CH₂—CH₂—O—CH₂—CH₂—O—CH₂—CH₂—O—CH₂—CH₂—C(═O)]₁₋₃— └IIIf┘
—HN—CH₂—CH₂—[O—CH₂—CH₂]₂₋₁₂—O—CH₂—C(═O)— [IIIg]
—HN—CH₂—CH₂—[O—CH₂—CH₂]₄₋₁₂—O—CH₂—CH₂—C(═O)— └IIIh┘
Z¹ is absent or represents Gly, β-Ala, Ser, D-Ser, Thr, D-Thr, His, D-His, Asn, D-Asn, Gln, D-Gln, Glu, D-Glu, Asp, D-Asp, Ala, D-Ala, Pro, D-Pro, Hyp or D-Hyp;
Z² is absent or represents Gly, β-Ala, Ser, D-Ser, Thr, D-Thr, His, D-His, Asn, D-Asn, Gln, D-Gln, Glu, D-Glu, Asp, D-Asp, Ala, D-Ala, Pro, D-Pro, Hyp or D-Hyp;
Z³ represents Ser, D-Ser, Thr, D-Thr, His, D-His, Asn, D-Asn, Gln, D-Gln, Glu, D-Glu, Asp, D-Asp, Ala, D-Ala, Pro, D-Pro, Hyp or D-Hyp;
Z⁴ represents Gly, Ala, Pro, Hyp, Ser, homoSer, Thr, Tyr, Gln, Asn, 2-PyAla, 3-PyAla, 4-PyAla, His, homoArg, Arg, Lys, Dab, Dap or Orn;
Z⁵ represents Gly, Ala, Pro, Hyp, Ser, homoSer, Thr, Gln, Asn, 2-PyAla, 3-PyAla, 4-PyAla, His, homoArg, Arg, Lys, Dab, Dap or Orn;
Z⁶ represents Ala, D-Ala, Val, D-Val, Leu, D-Leu, Ile, D-Ile, Met, D-Met, Nle or D-Nle;
X¹ represents Glu, Asp, Cys, homoCys, Lys, Orn, Dab or Dap;
X² represents His, Cit, Dab, Dap, Cgl, Cha, Val, Ile, tBuGly, Leu, Tyr, Glu, Ala, Nle, Met, Met(O), Met(O₂), Gln, Gln(alkyl), Gln(aryl), Asn, Asn(alkyl), Asn(aryl), Ser, Thr, Cys, Pro, Hyp, Tic, 2-PyAla, 3-PyAla, 4-PyAla, (2-thienyl)alanine, 3-(thienyl)alanine, (4-thiazolyl)Ala, (2-furyl)alanine, (3-furyl)alanine or Phe, wherein one or more hydrogens on the phenyl moiety of said Phe may optionally and independently be substituted by a substituent selected among halogen, hydroxy, alkoxy, nitro, benzoyl, methyl, trifluoromethyl, amino and cyano;
X³ represents D-Phe, wherein one or more hydrogens on the phenyl moiety in D-Phe may optionally and independently be substituted by a substituent selected among halogen, hydroxy, alkoxy, nitro, methyl, trifluoromethyl and cyano;
X⁴ represents Trp, 2-Nal, (3-benzo[b]thienyl)alanine or (S)-2,3,4,9-tetrahydro-1H-β-carboline-3-carboxylic acid;
X⁵ represents Glu, Asp, Cys, homoCys, Lys, Orn, Dab or Dap;
wherein X¹ and X⁵ are joined, rendering the compound of formula II cyclic, either via a disulfide bridge deriving from X¹ and X⁵ both independently being Cys or homoCys, or via an amide bond formed between a carboxylic acid in the side-chain of X¹ and an amino group in the side-chain of X⁵, or between a carboxylic acid in the side-chain of X⁵ and an amino group in the side-chain of X¹;
R⁴ represents OR′ or N(R′)₂, wherein each R′ independently represents hydrogen or represents C₁₋₆alkyl, C₂₋₆alkenyl or C₂₋₆alkynyl which may optionally be substituted with one or more amino or hydroxy;
or a pharmaceutically acceptable salt, prodrug or solvate thereof,
in combination with bariatric surgical intervention,
for treating a patient in need thereof in order to achieve weight loss or prevent weight gain in said patient.

4. A pharmaceutical composition comprising a compound according to formula IVa, IVb or IVc:
R¹—R²—C(═O)—R³—S²-Z⁴-Z⁵-Z⁶-c[X¹—X²—X³-Arg-X⁴—X⁵]R⁴ [IVa]
R¹—R²—C(═O)—R³—S²-Z⁵-Z⁶-c└X¹—X²—X³-Arg-X⁴—X⁵┘R⁴ └IVb┘
R¹—R²—C(═O)—R³—S²-Z⁶-c[X¹—X²—X³-Arg-X⁴—X⁵]R⁴ [IVc]
wherein
R¹ represents tetrazol-5-yl or carboxy;
R² represents a straight-chain, branched and/or cyclic C₆₋₂₀alkyl, C₆₋₂₀alkenyl or C₆₋₂₀alkynyl which may optionally be substituted with one or more substituents selected from halogen, hydroxyl and aryl;
R³ is absent or represents —NH—S(═O)₂—(CH₂)₃₋₅—C(═O)— or a peptide fragment comprising one or two amino acid residues and containing at least one carboxy group;
S² represents a glycolether-based structure according to one of the formulas IIIa-IIIh;
—HN—CH₂—CH₂—O—CH₂—CH₂—O—CH₂—C(═O)— [IIIa]
—[HN—CH₂—CH₂—O—CH₂—CH₂—O—CH₂—C(═O)]₂— [IIIb]
—[HN—CH₂—CH₂—O—CH₂—CH₂—O—CH₂—C(═O)]₃₋₅— [IIIc]
—[HN—CH₂—CH₂—O—CH₂—CH₂—O—CH₂—CH₂—NH—C(═O)—CH₂—CH₂—CH₂—C(═O)]₁₋₃— └IIId┘
—[HN—CH₂—CH₂—O—CH₂—CH₂—O—CH₂—CH₂—NH—C(═O)—CH₂—O—CH₂—C(═O)]₁₋₃— [IIIe]
—[HN—CH₂—CH₂—O—CH₂—CH₂—O—CH₂—CH₂—O—CH₂—CH₂—O—CH₂—CH₂—C(═O)]₁₋₃— [IIIf]
—HN—CH₂—CH₂—[O—CH₂—CH₂]₂₋₁₂—O—CH₂—C(═O)— [IIIg]
—HN—CH₂—CH₂—[O—CH₂—CH₂]₄₋₁₂—O—CH₂—CH₂—C(═O)— [IIIh]
Z⁴ represents Gly, Ala, Pro, Hyp, Ser, homoSer, Thr, Tyr, Gln, Asn, 2-PyAla, 3-PyAla, 4-PyAla, His, homoArg, Arg, Lys, Dab, Dap or Orn;
Z⁵ represents Gly, Ala, Pro, Hyp, Ser, homoSer, Thr, Gln, Asn, 2-PyAla, 3-PyAla, 4-PyAla, His, homoArg, Arg, Lys, Dab, Dap or Orn;
Z⁶ represents Ala, D-Ala, Val, D-Val, Leu, D-Leu, Ile, D-Ile, Met, D-Met, Nle or D-Nle;
X¹ represents Glu, Asp, Cys, homoCys, Lys, Orn, Dab or Dap;
X² represents His, Cit, Dab, Dap, Cgl, Cha, Val, Ile, tBuGly, Leu, Tyr, Glu, Ala, Nle, Met, Met(O), Met(O₂), Gln, Gln(alkyl), Gln(aryl), Asn, Asn(alkyl), Asn(aryl), SerThr, Cys, Pro, Hyp, Tic, 2-PyAla, 3-PyAla, 4-PyAla, (2-thienyl)alanine, 3-(thienyl)alanine, (4-thiazolyl)Ala, (2-furyl)alanine, (3-furyl)alanine or Phe, wherein one or more hydrogens on the phenyl moiety of said Phe may optionally and independently be substituted by a substituent selected among halogen, hydroxy, alkoxy, nitro, benzoyl, methyl, trifluoromethyl, amino and cyano;
X³ represents D-Phe, wherein one or more hydrogens on the phenyl moiety in D-Phe may optionally and independently be substituted by a substituent selected among halogen, hydroxy, alkoxy, nitro, methyl, trifluoromethyl and cyano;
X⁴ represents Trp, 2-Nal, (3-benzo[b]thienyl)alanine or (S)-2,3,4,9-tetrahydro-1H-β-carboline-3-carboxylic acid;
X⁵ represents Glu, Asp, Cys, homoCys, Lys, Orn, Dab or Dap;
wherein X¹ and X⁵ are joined, rendering the compound of formula IVa, IVb or IVc cyclic, either via a disulfide bridge deriving from X¹ and X⁵ both independently being Cys or homoCys, or via an amide bond formed between a carboxylic acid in the side-chain of X¹ and an amino group in the side-chain of X⁵ or between a carboxylic acid in the side-chain of X⁵ and an amino group in the side-chain of X¹;
R⁴ represents OR′ or N(R′)₂, wherein each R′ independently represents hydrogen or represents C₁₋₆alkyl, C₂₋₆alkenyl or C₂₋₆alkynyl which may optionally be substituted with one or more amino or hydroxy;
or a pharmaceutically acceptable salt, prodrug or solvate thereof,
in combination with bariatric surgical intervention,
for treating a patient in need thereof in order to achieve weight loss or prevent weight gain in said patient.

5. The composition according to claim 3, wherein the compound is selected from the group consisting of:
16-(Tetrazol-5-yl)hexadecanoyl-Gly-Thr-Gln-His-Ser-Nle-c└Glu-Hyp-D-Phe-Arg-Trp-Lys]-NH₂ 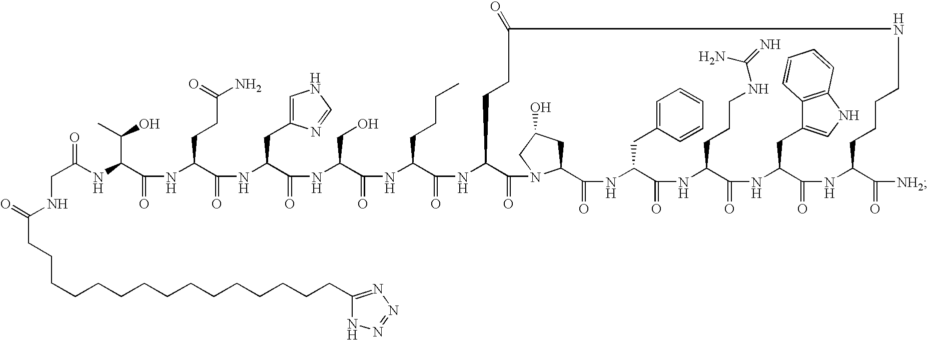
16-(Tetrazol-5-yl)hexadecanoyl-Gly-Thr-Gln-Dap-Ser-Nle-c[Glu-Hyp-D-Phe-Arg-Trp-Lys]-NH₂ 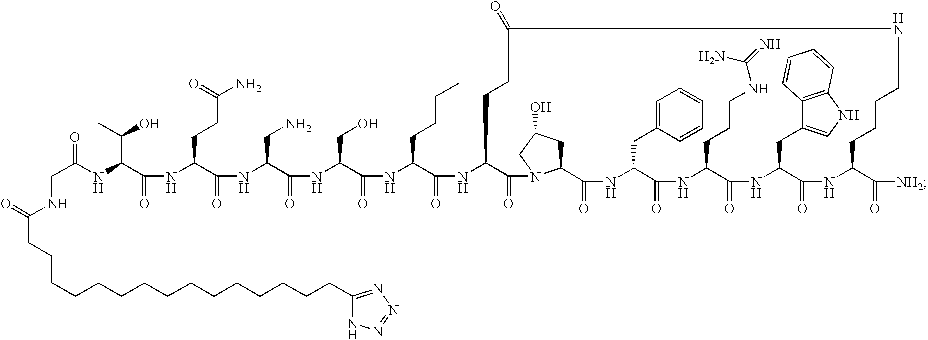
{2-[2-(16-(Tetrazol-5-yl)hexadecanoylamino)ethoxy]ethoxy}acetyl-Gly-Thr-Gln-Dap-Ser-Nle-c[Glu-Hyp-D-Phe-Arg-Tip-Lys]-NH₂ 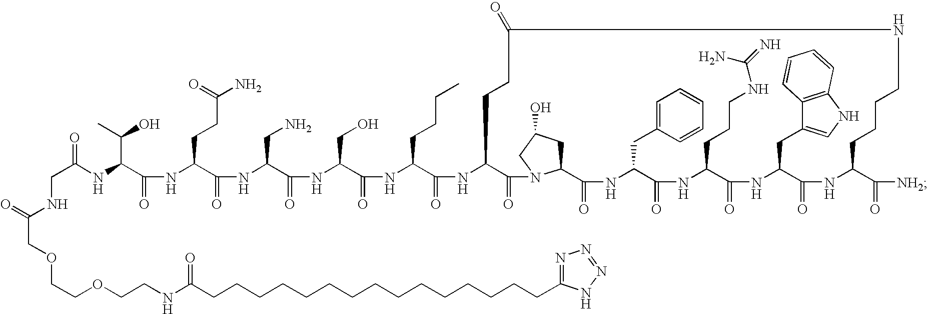
{2-[2-(16-(Tetrazol-5-yl)hexadecanoylamino)ethoxy]ethoxy}acetyl-Gly-Ser-Gln-His-Ser-Nle-c[Glu-Dap-D-Phe-Arg-Trp-Lys]-NH₂ 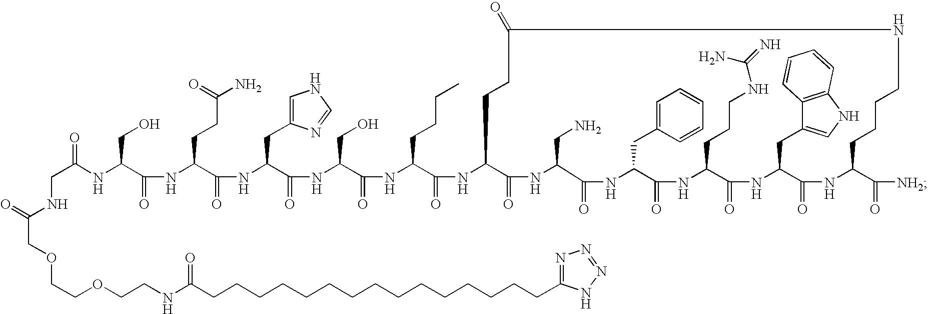
{2-[2-(16-(Tetrazol-5-yl)hexadecanoylamino)ethoxy]ethoxy}acetyl-Gly-Ser-Gln-His-Dap-Nle-c[Glu-Hyp-D-Phe-Arg-Tip-Lys]-NH₂ 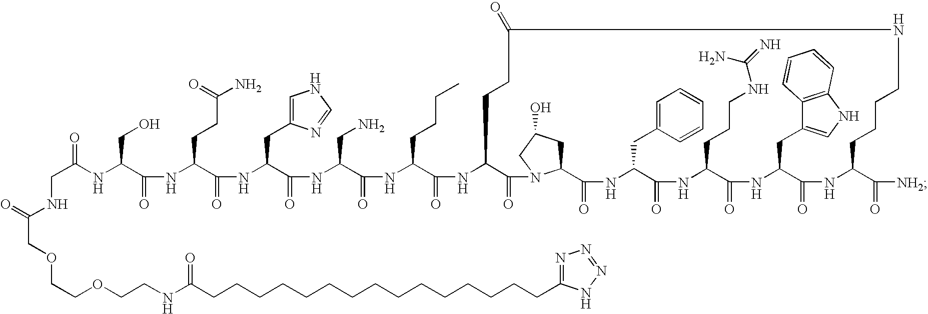
4-(16-(Tetrazol-5-yl)hexadecanoylsulfamoyl)butanoyl-Gly-Ser-D-Gln-His-Dap-Nle-c[Glu-Hyp-D-Phe-Arg-Tip-Lys]-NH₂ 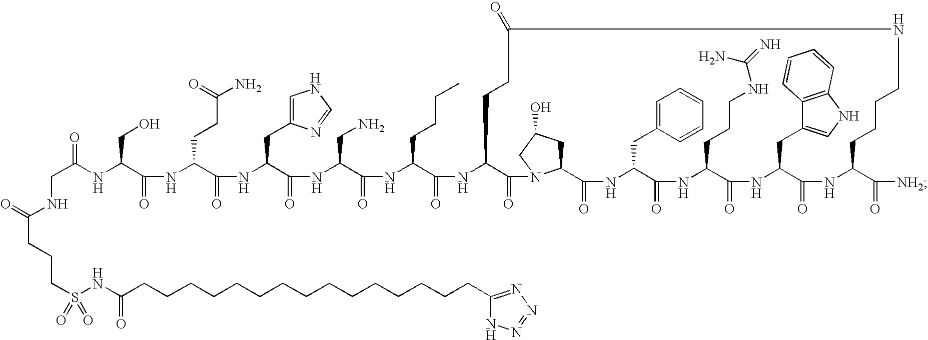
{2-[2-(2-{2-[2-(16-(Tetrazol-5-yl)hexadecanoylamino)ethoxy]ethoxy}acetylamino)ethoxy]-ethoxy}acetyl-Nle-c[Glu-Dap-D-Phe-Arg-Trp-Lys]-NH₂ 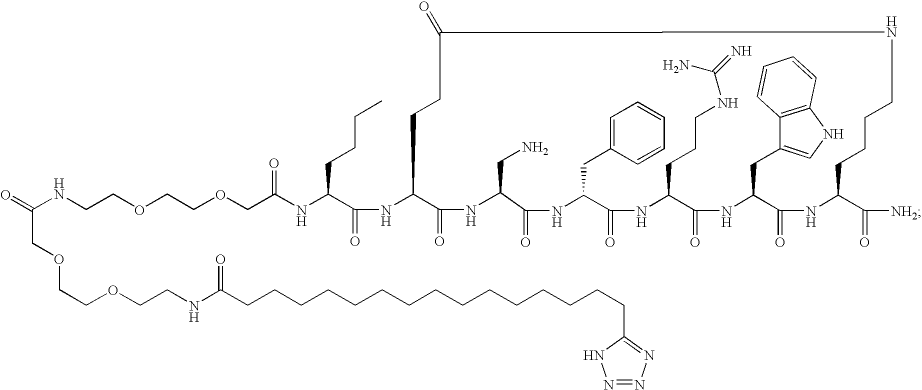
(2-{2-[4-(16-(Tetrazol-5-yl)hexadecanoylsulfamoyl)butanoylamino]ethoxy}ethoxy)acetyl-His-Nle-c[Glu-Dap-D-Phe-Arg-Trp-Lys]-NH₂ 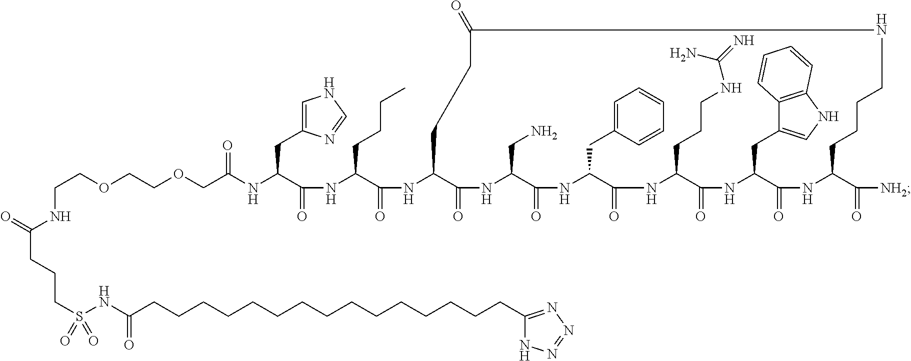
{2-[2-(2-{2-[2-(15-Carboxypentadecanoylamino)ethoxy]ethoxy}acetylamino)ethoxy]ethoxy}-acetyl-Pro-Nle-c[Glu-Hyp-D-Phe-Arg-Trp-Lys]-NH₂ 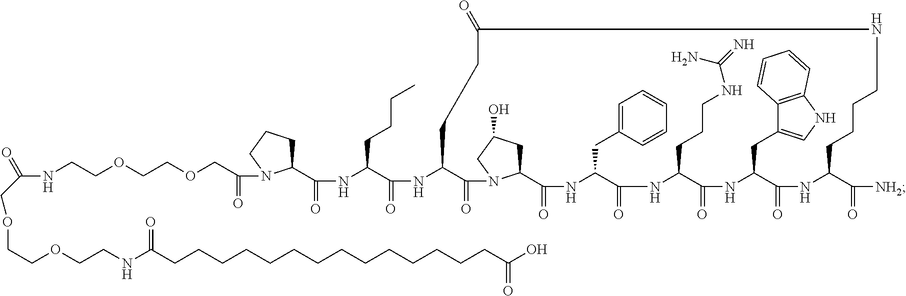
{2-[2-(2-{2-[2-(15-Carboxypentadecanoylamino)ethoxy]ethoxy}acetylamino)ethoxy]ethoxy}-acetyl-Nle-c[Glu-Hyp-D-Phe-Arg-Trp-Lys]-NH₂ 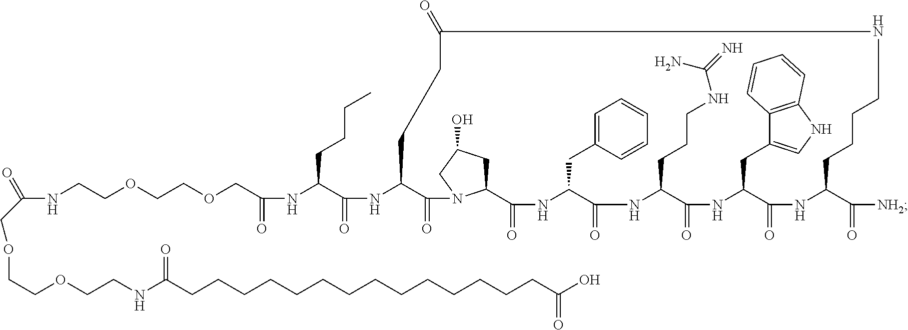
the compound: 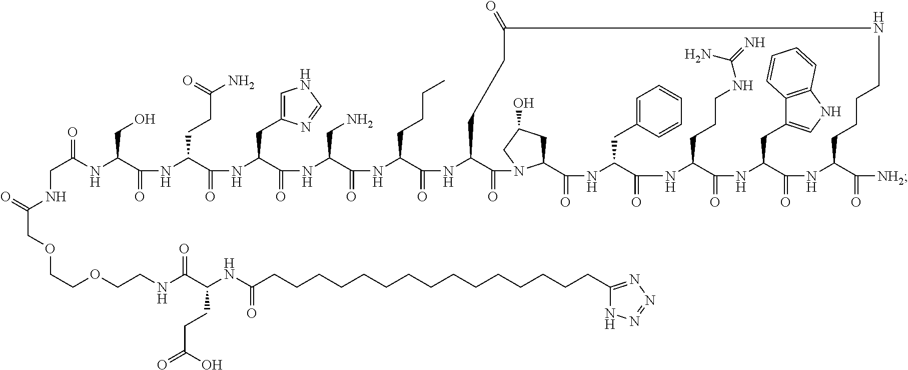
the compound: 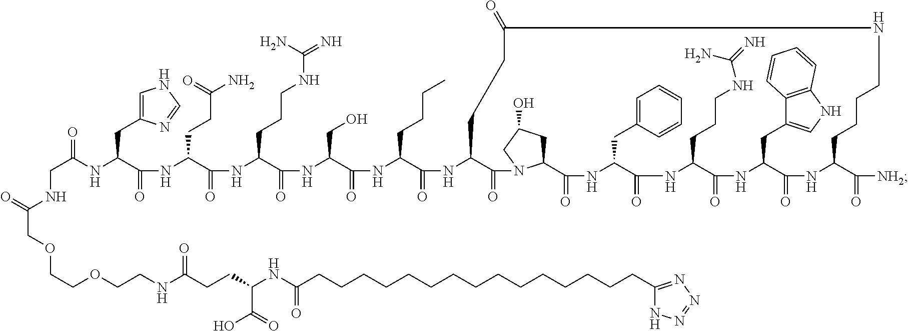
the compound: 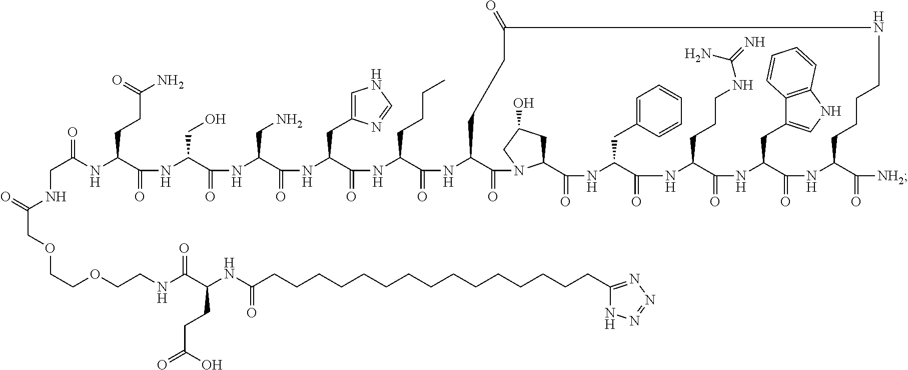
the compound: 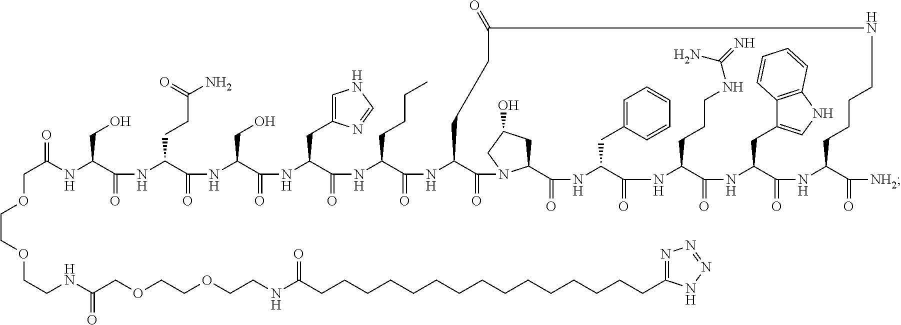
the compound: 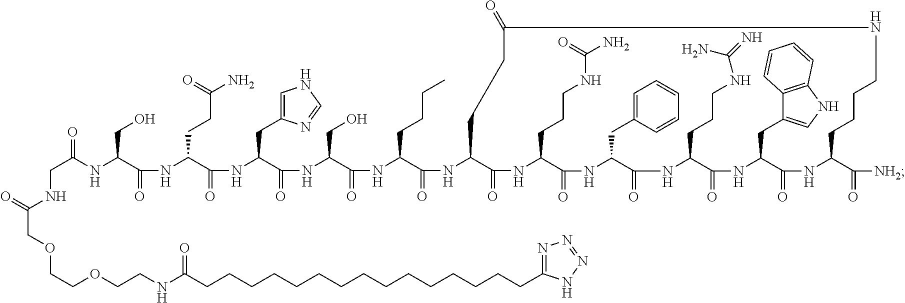
the compound: 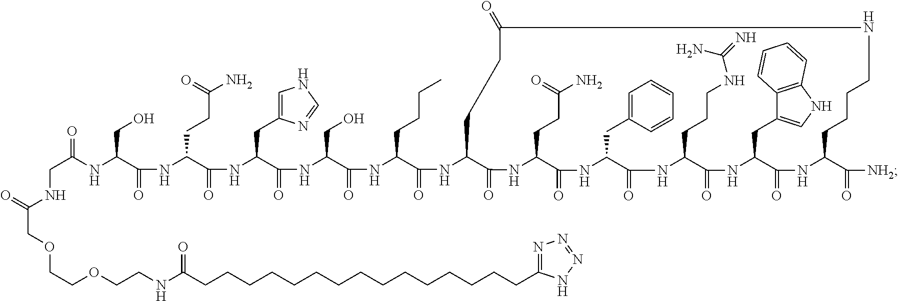
(2-{2-[2-(2-{2-[(R)-4-Carboxy-2-(16-(1H-tetrazol-5-yl)hexadecanoylamino)butanoylamino]-ethoxy}ethoxy)acetylamino]ethoxy}ethoxy)acetyl-Ser-Ser-Nle-c[Glu-Hyp-D-Phe-Arg-Trp-Lys]-NH₂ 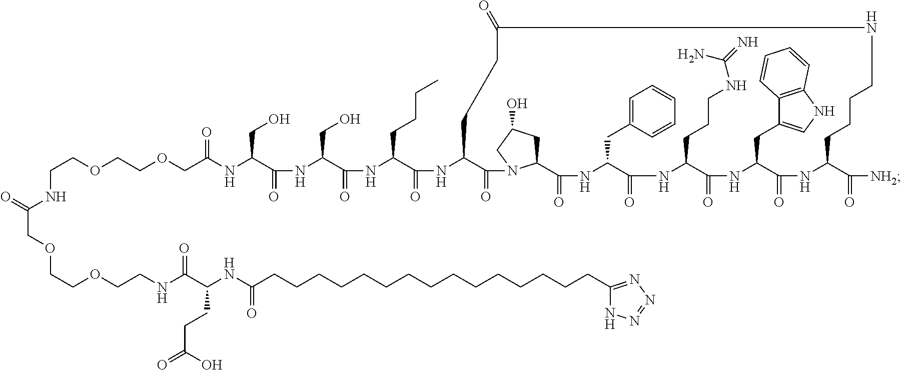
the compound: 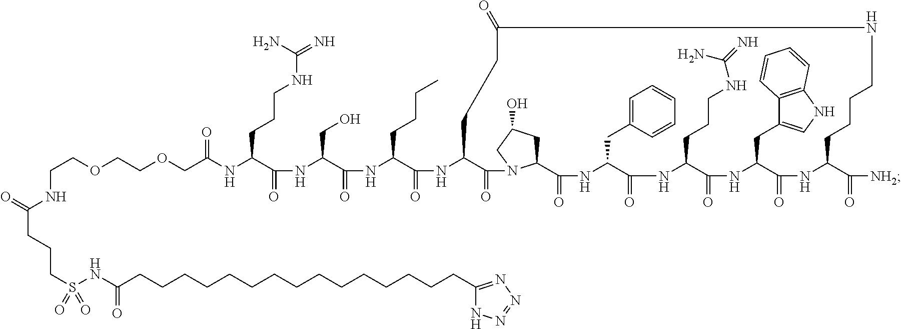
the compound: 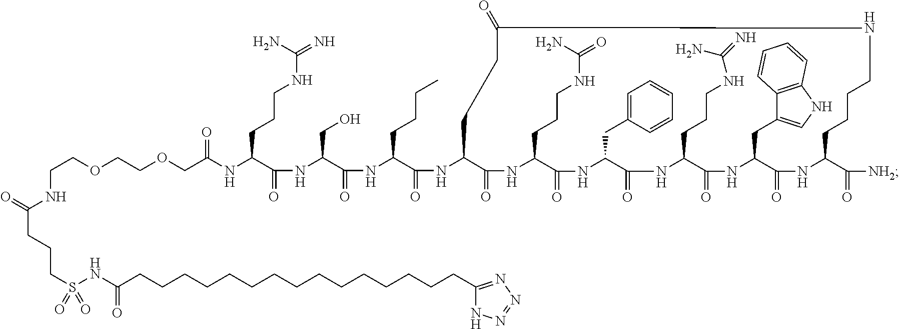
{2-[2-(15-(Carboxy)pentadecanoylamino)ethoxy]ethoxy}acetyl-Gly-Ser-Gln-His-Dap-Nle-c[Glu-Hyp-D-Phe-Arg-Tip-Lys]-NH₂ 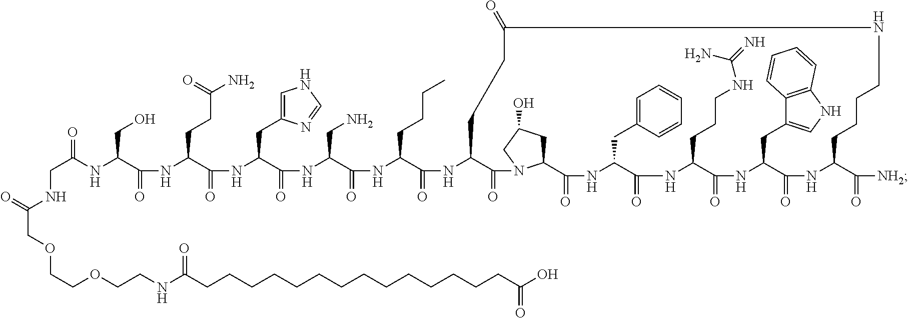
the compound: 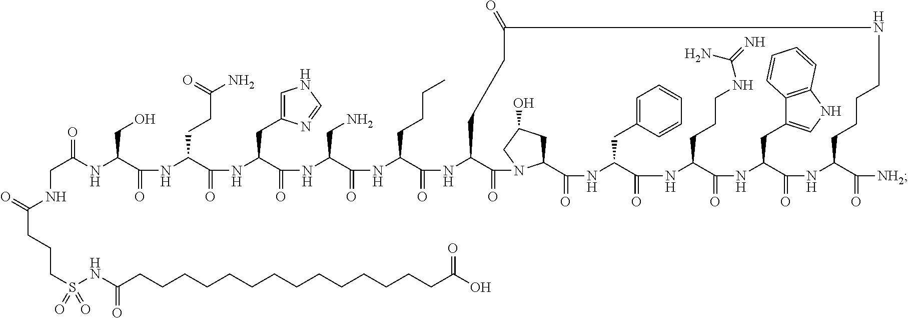
the compound: 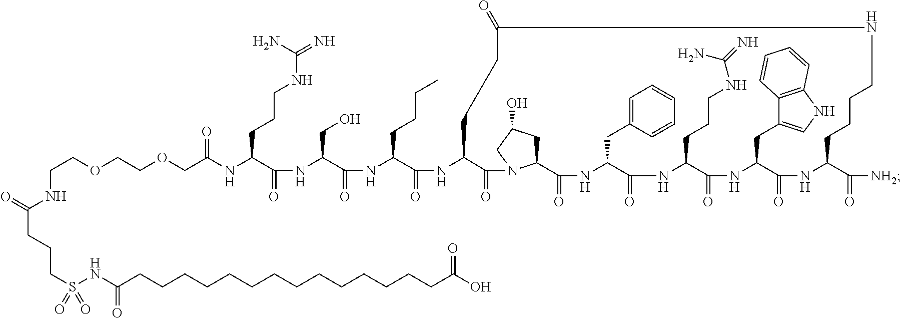
the compound: 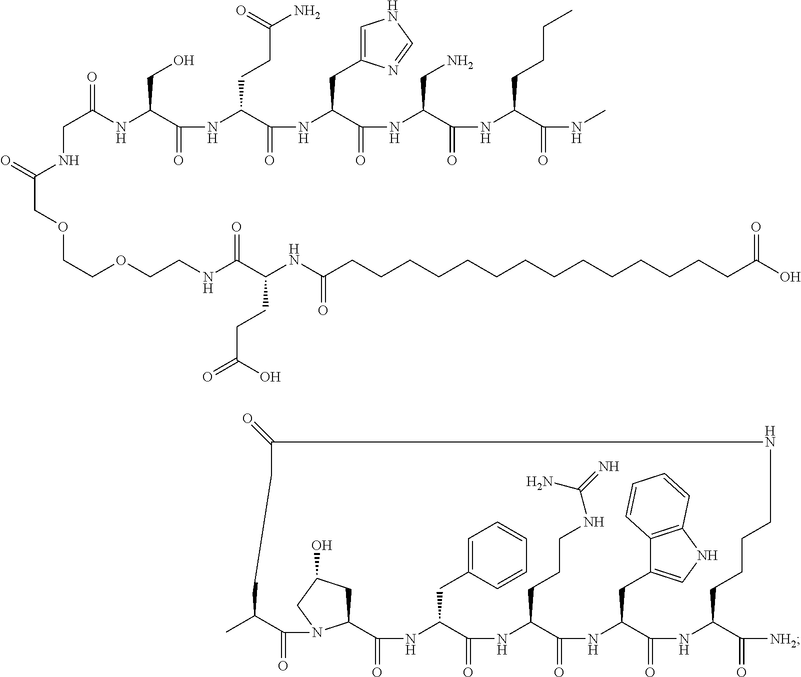
15-Carboxypentadecanoyl-Gly-Ser-Ser-Tyr-Thr-Nle-c[Glu-Hyp-D-Phe-Arg-Tip-Lys]-NH₂ 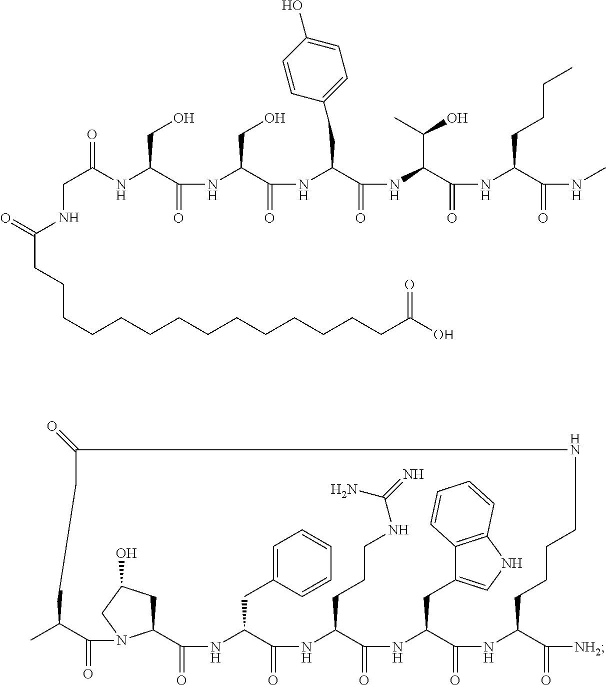
{2-└2-(15-Carboxypentadecanoylamino)ethoxy]ethoxy}acetyl-Ser-Tyr-Hyp-Nle-c[Glu-Hyp-D-Phe-Arg-Tip-Lys]-NH₂ 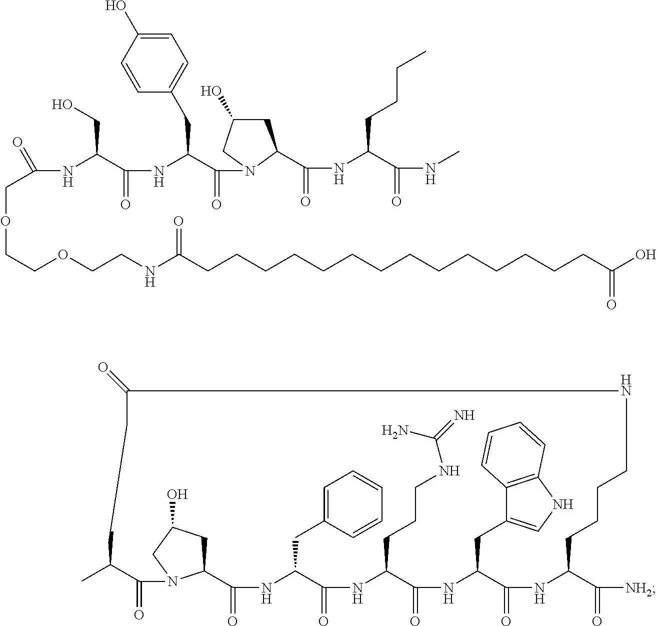
{2-[2-(15-Carboxypentadecanoylamino)ethoxy]ethoxy}acetyl-Asn-Asn-Pro-Nle-c[Glu-Hyp-D-Phe-Arg-Tip-Lys]-NH₂ 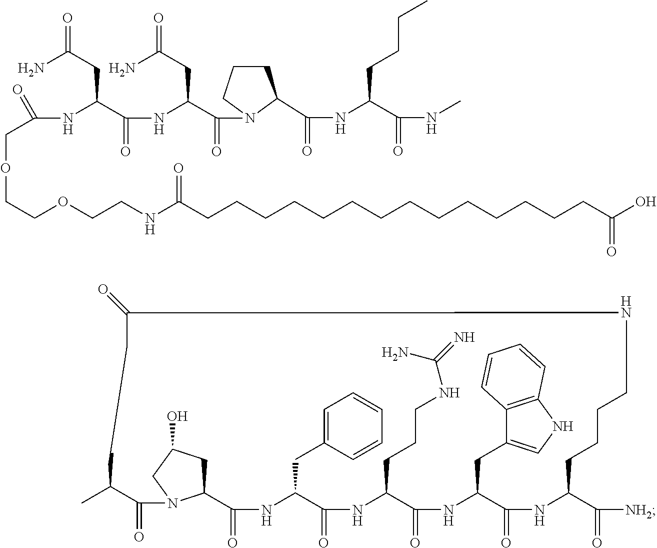
(2-{2-[(R)-4-Carboxy-2-(16-(tetrazol-5-yl)hexadecanoylamino)butanoylamino]ethoxy}-ethoxy)acetyl-Gly-Ser-Gln-His-Dap-Nle-c[Glu-Hyp-D-Phe-Arg-Trp-Lys]-NH₂ 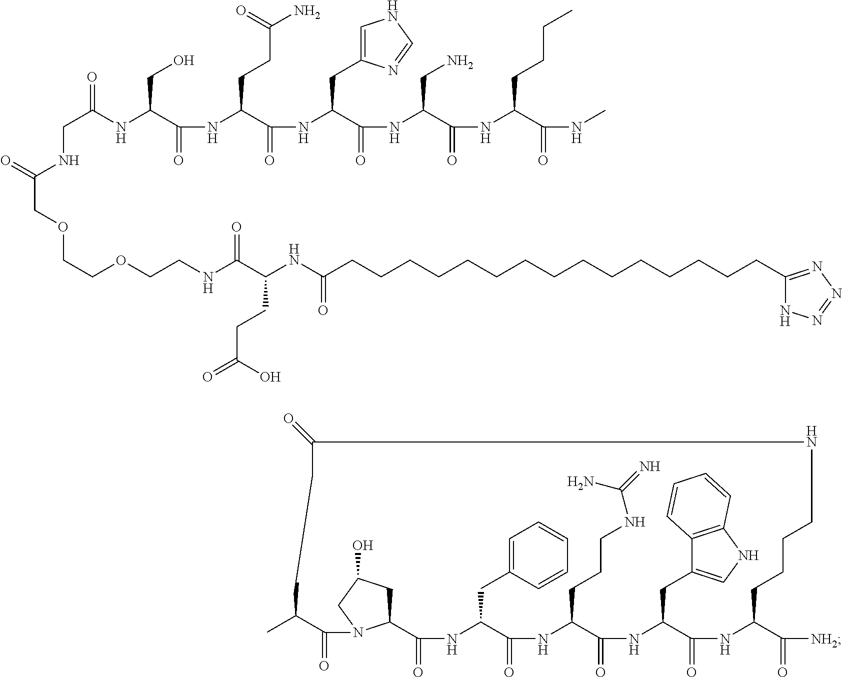
{2-[2-(2-{2-[2-(16-(Tetrazol-5-yl)hexadecanoylamino)ethoxy]ethoxy}acetylamino)ethoxy]-ethoxy}acetyl-Ser-Gln-His-Dap-Nle-c[Glu-Hyp-D-Phe-Arg-Trp-Lys]-NH₂ 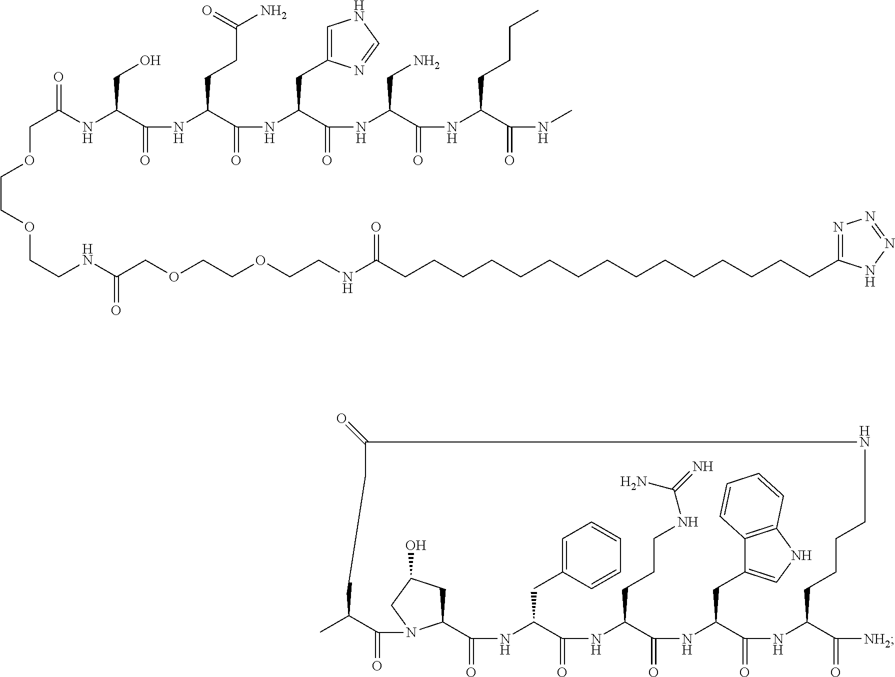
(2-{2-[4-(16-(Tetrazol-5-yl)hexadecanoylsulfamoyl)butanoylamino]ethoxy}ethoxy)acetyl-Arg-Dap-Nle-c[Glu-Hyp-D-Phe-Arg-Trp-Lys]-NH₂ 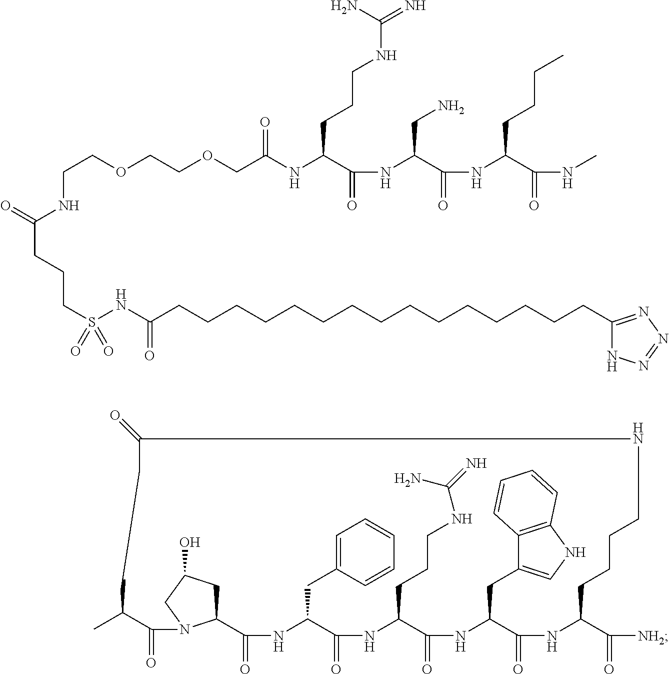
{2-[2-(16-(Tetrazol-5-yl)hexadecanoylamino)ethoxy]ethoxy}acetyl-Gly-Ser-Gln-Dap-Ser-Nle-c[Glu-Hyp-D-Phe-Arg-Trp-Lys]-NH₂ 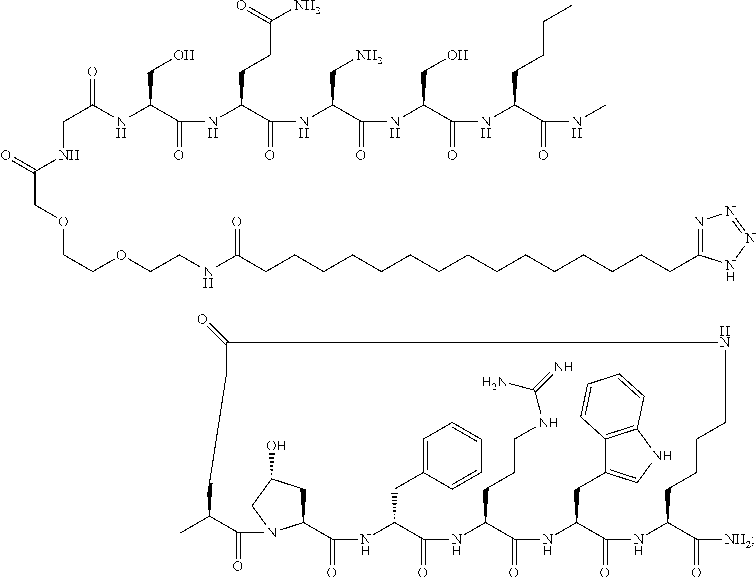
(2-{2-[4-(16-(Tetrazol-5-yl)hexadecanoylsulfamoyl)butanoylamino]ethoxy}ethoxy)acetyl-His-Dap-Nle-c[Glu-Hyp-D-Phe-Arg-Trp-Lys]-NH₂ 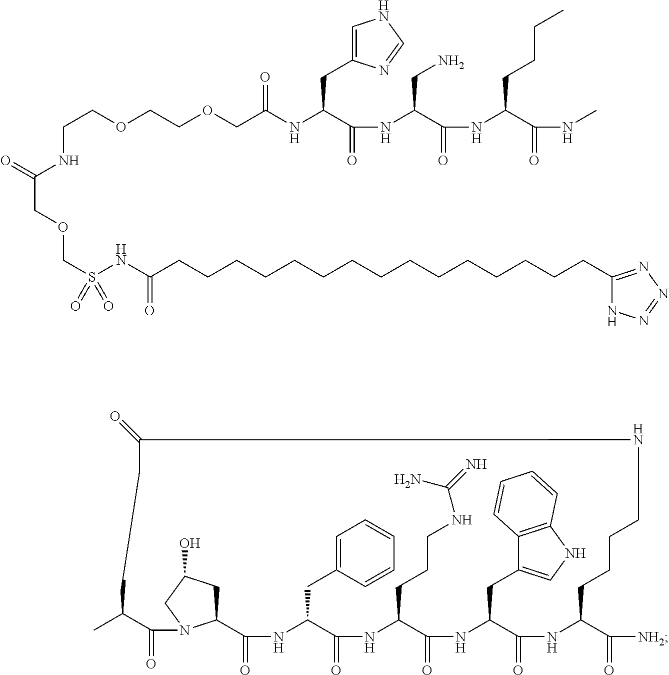
{2-[2-(2-{2-[2-(16-(Tetrazol-5-yl)hexadecanoylamino)ethoxy]ethoxy}acetylamino)ethoxy]ethoxy}acetyl-Dap-Nle-c[Glu-Hyp-D-Phe-Arg-Trp-Lys]-NH₂ 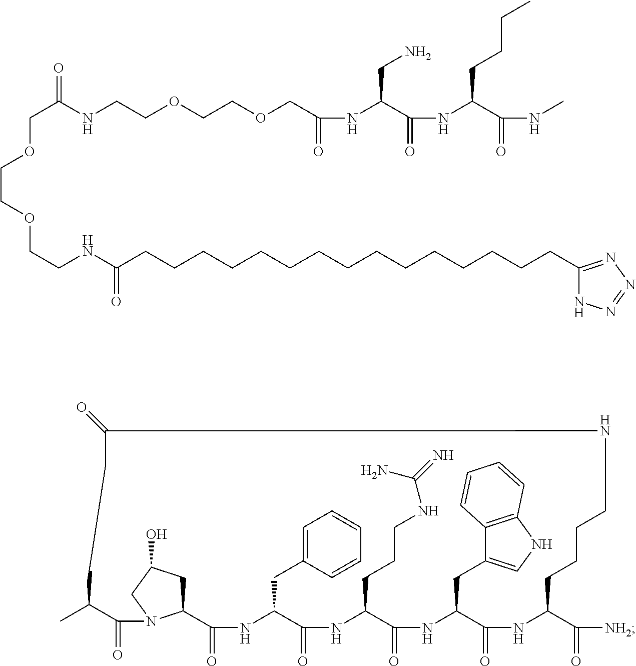
(2-{2-[4-(16-(Tetrazol-5-yl)hexadecanoylsulfamoyl)butanoylamino]ethoxy}ethoxy)acetyl-Gly-Ser-Gln-His-Dap-Nle-c[Glu-Hyp-D-Phe-Arg-Trp-Lys]-NH₂ 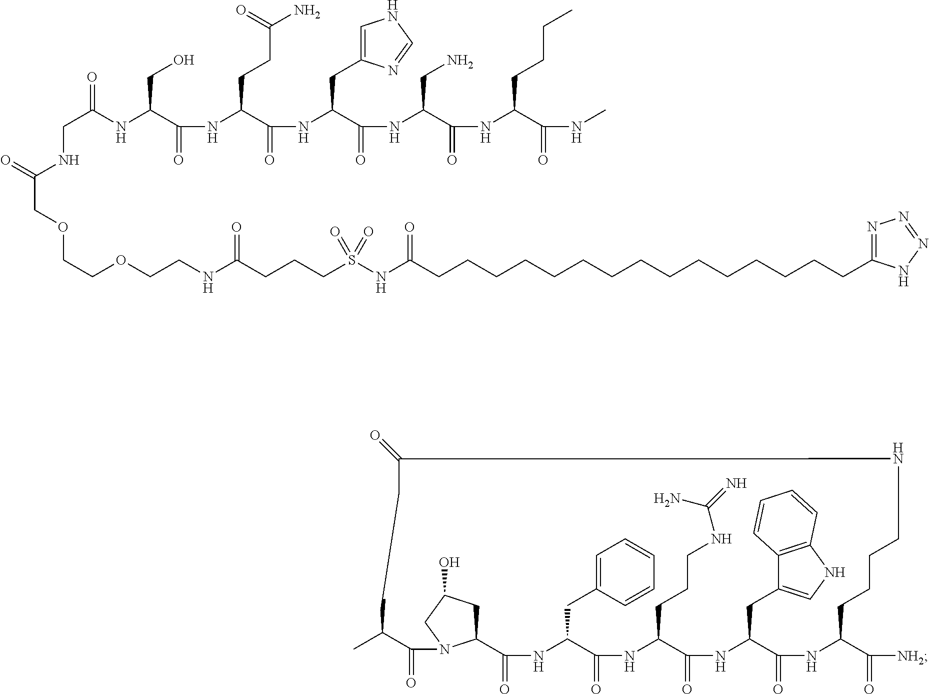
4-(16-(Tetrazol-5-yl)hexadecanoylsulfamoyl)butanoyl-Gly-Ser-Gln-His-Dap-Nle-c[Glu-Hyp-D-Phe-Arg-Tip-Lys]-NH₂ 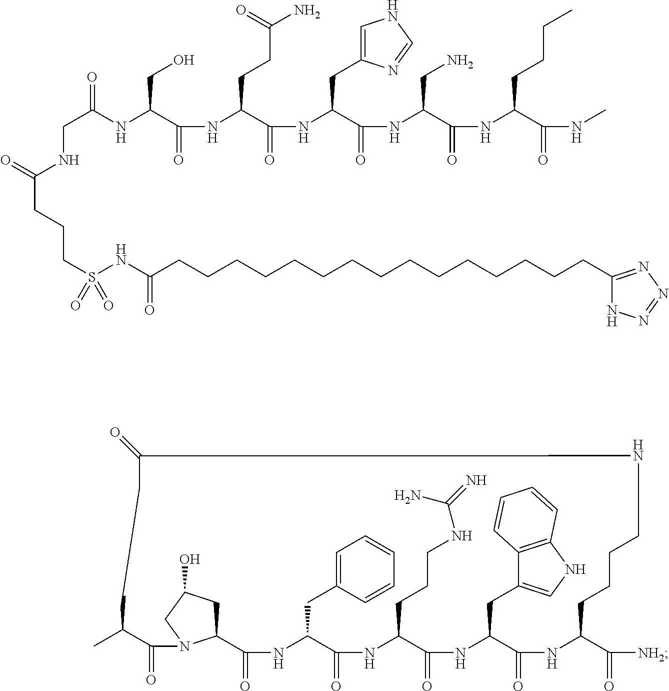
{2-[2-(16-(Tetrazol-5-yl)hexadecanoylamino)ethoxy]ethoxy}acetyl-Gly-Ser-D-Ser-His-His-Nle-c[Glu-Hyp-D-Phe-Arg-Trp-Lys]-OH 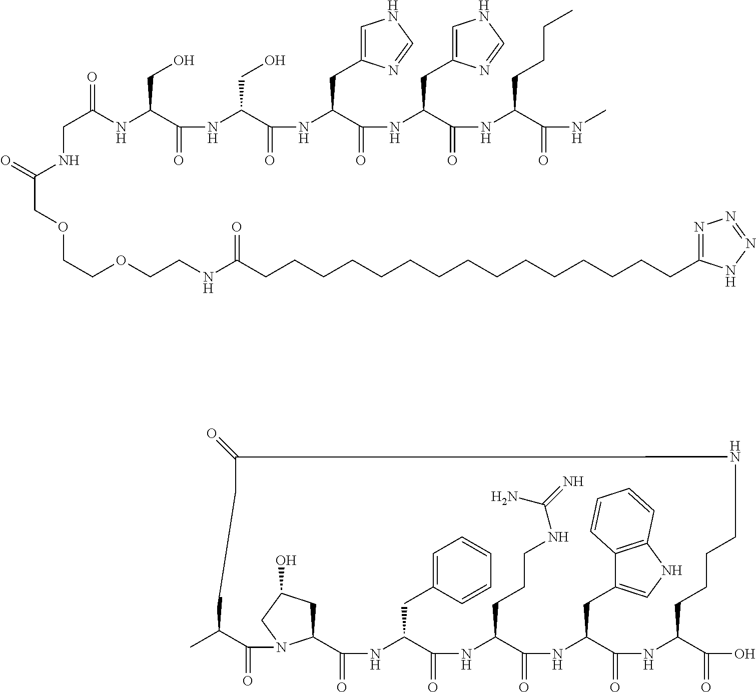
(2-[2-{(2-[2-{16-(Tetrazol-5-yl)hexadecanoylamino}ethoxy]ethoxy)acetylamino}ethoxy]-ethoxy)acetyl-Gly-Ser-Gln-His-Dap-Nle-c[Glu-Hyp-D-Phe-Arg-Trp-Lys]-NH₂ 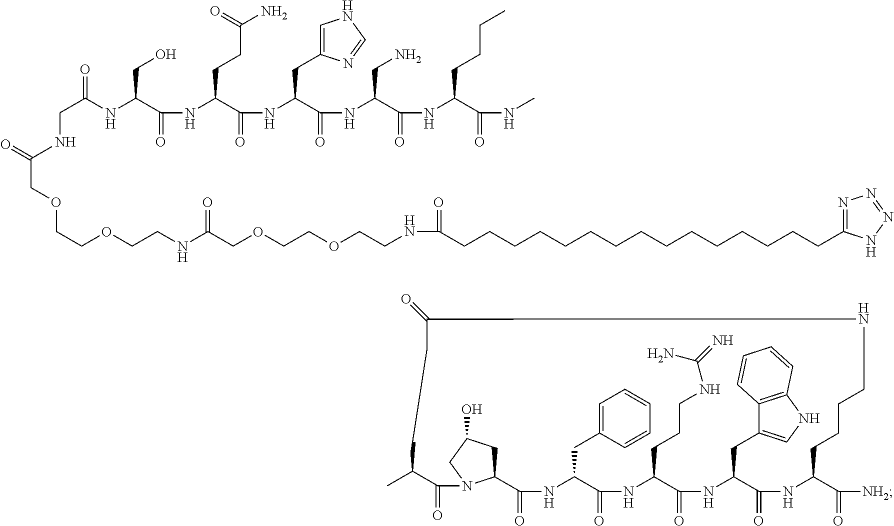
(2-[2-{(2-[2-{(2-[2-{(2-[2-{16-(Tetrazol-5-yl)hexadecanoylamino}ethoxy]ethoxy)acetylamino}-ethoxy]ethoxy)acetylamino}ethoxy]ethoxy)acetylamino}ethoxy]ethoxy)acetyl-Gly-Ser-Gln-His-Dap-Nle-c[Glu-Hyp-D-Phe-Arg-Trp-Lys]-NH₂ 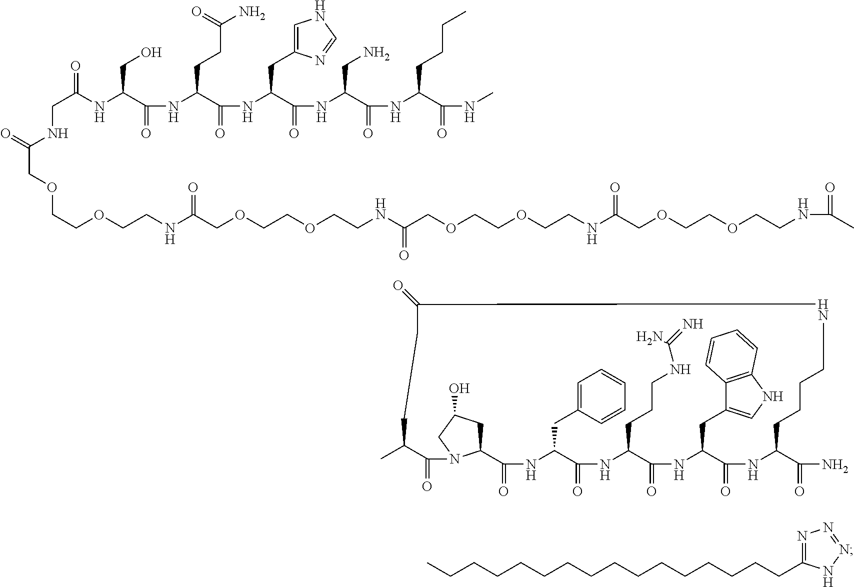
{2-[2-(16-(Tetrazol-5-yl)hexadecanoylamino)ethoxy]ethoxy}acetyl-Gly-Ser-Gln-His-His-Nle-c[Glu-Hyp-D-Phe-Arg-Tip-Lys]-NH₂ 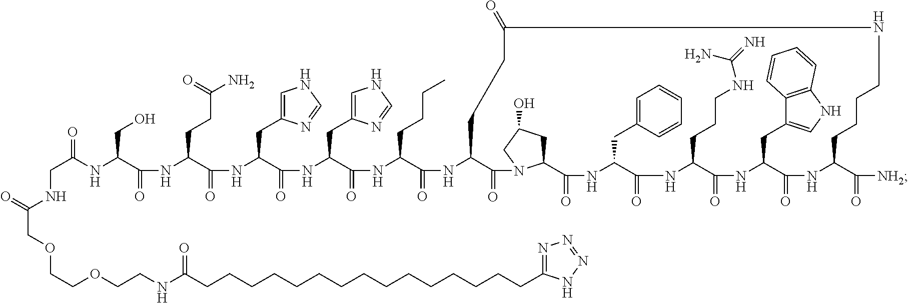
{2-[2-(16-(Tetrazol-5-yl)hexadecanoylamino)ethoxy]ethoxy}acetyl-Gly-Ser-Gln-His-Ser-Nle-c[Glu-Hyp-D-Phe-Arg-Tip-Lys]-NH₂ 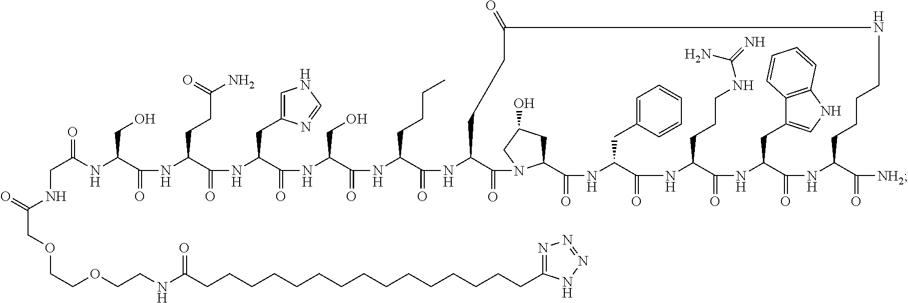
{2-[2-(16-(Tetrazol-5-yl)hexadecanoylamino)ethoxy]ethoxy}acetyl-Gly-Ser-Gln-His-Nle-c[Glu-Hyp-D-Phe-Arg-Tip-Lys]-NH₂ 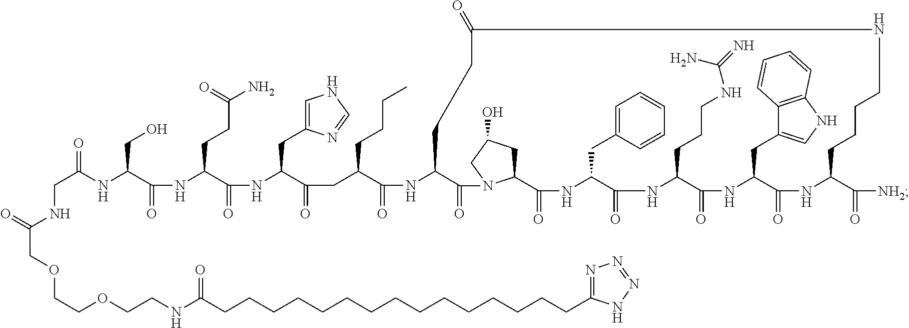
4-(15-Carboxypentadecanoylsulfamoyl)butanoyl-Gly-Ser-Gln-His-Dap-Nle-c[Glu-Hyp-D-Phe-Arg-Trp-Lys]-NH₂ 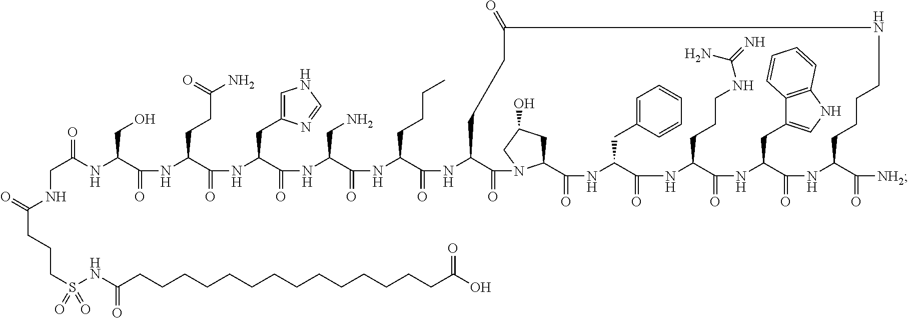
(2-{2-[(S)-4-Carboxy-4-(17-carboxyheptadecanoylamino)butanoylamino]ethoxy}ethoxy)-acetyl-Gly-Ser-Gln-His-Dap-Nle-c[Glu-Hyp-D-Phe-Arg-Trp-Lys]-NH₂ 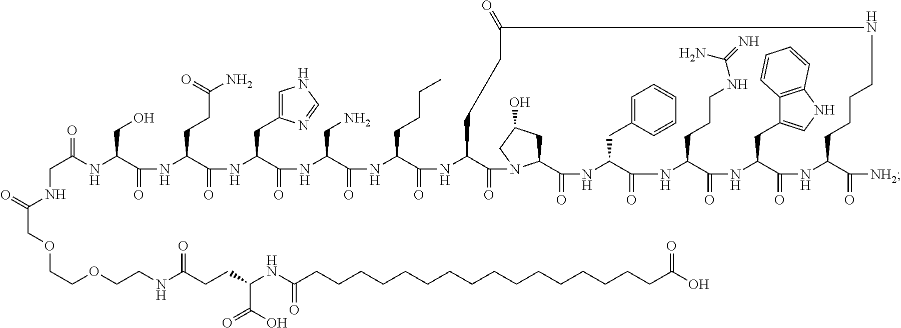
[2-(2-{(S)-4-Carboxy-4-[2-(17-carboxyheptadecanoylamino)acetylamino]butanoylamino}-ethoxy)ethoxy]acetyl-Gly-Ser-Gln-His-Dap-Nle-c[Glu-Hyp-D-Phe-Arg-Trp-Lys]-NH₂ 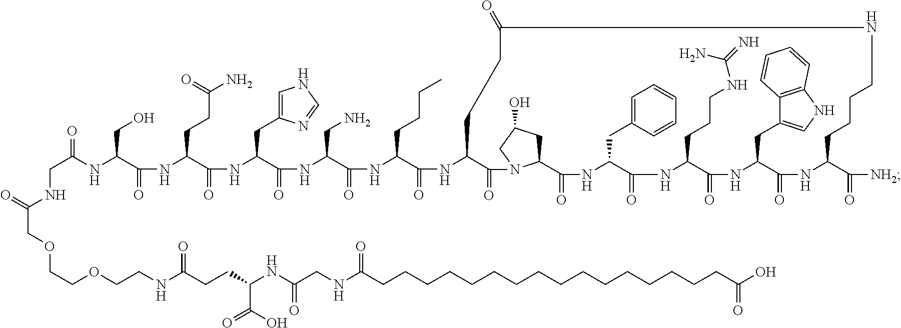
(2-{2-[(S)-3-Carboxy-3-(17-carboxyheptadecanoylamino)propanoylamino]ethoxy}ethoxy)-acetyl-Gly-Ser-Gln-His-Dap-Nle-c[Glu-Hyp-D-Phe-Arg-Trp-Lys]-NH₂ 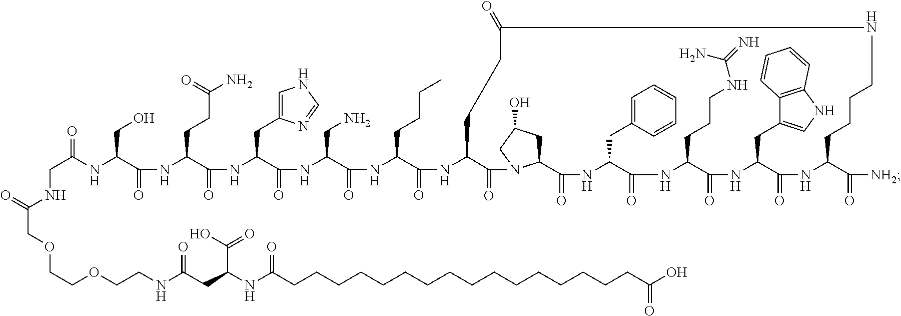
{2-[2-(16-(Tetrazol-5-yl)hexadecanoylamino)ethoxy]ethoxy}acetyl-Gly-Ser-Gln-His-Thr-Nle-c[Glu-Hyp-D-Phe-Arg-Trp-Lys]-NH₂ 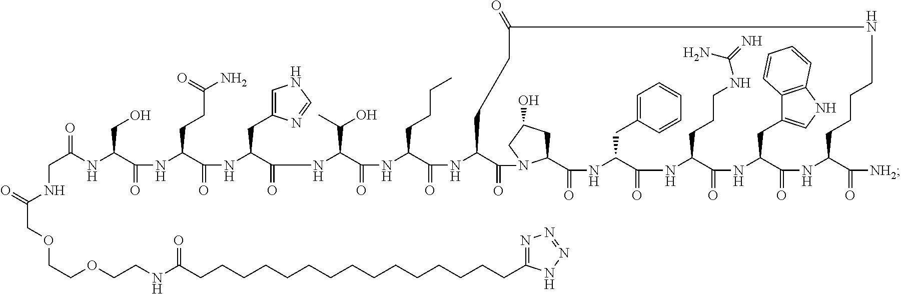
{2-[2-(16-(Tetrazol-5-yl)hexadecanoylamino)ethoxy]ethoxy}acetyl-Gly-Ser-Gln-His-Dab-Nle-c[Glu-Hyp-D-Phe-Arg-Tip-Lys]-NH₂ 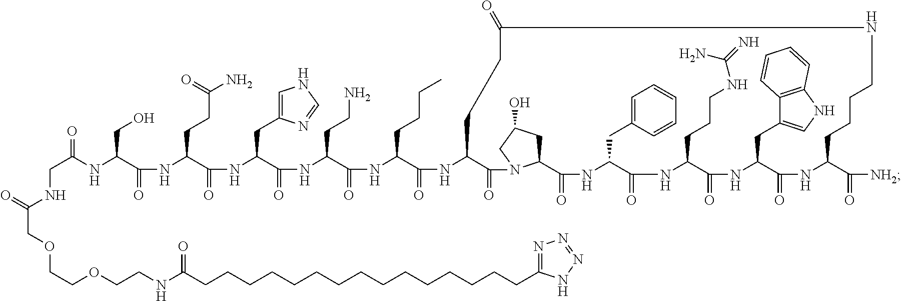
{2-└2-(16-(Tetrazol-5-yl)hexadecanoylamino)ethoxy]ethoxy}acetyl-Gly-Ser-Gln-His-homoSer-Nle-c[Glu-Hyp-D-Phe-Arg-Tip-Lys]-NH₂ 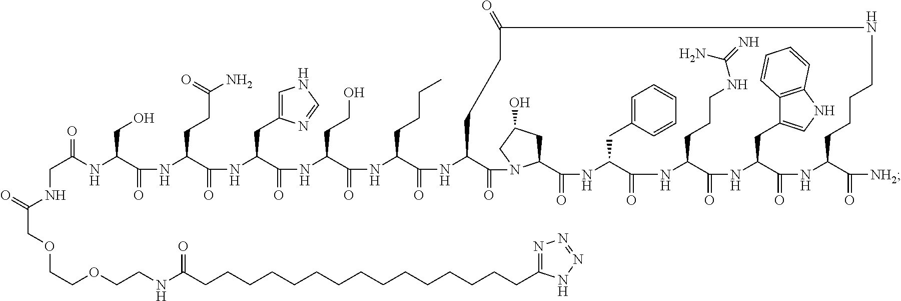
{2-[2-(16-(Tetrazol-5-yl)hexadecanoylamino)ethoxy]ethoxy}acetyl-Gly-Ser-Gln-His-Orn-Nle-c[Glu-Hyp-D-Phe-Arg-Trp-Lys]-NH₂ 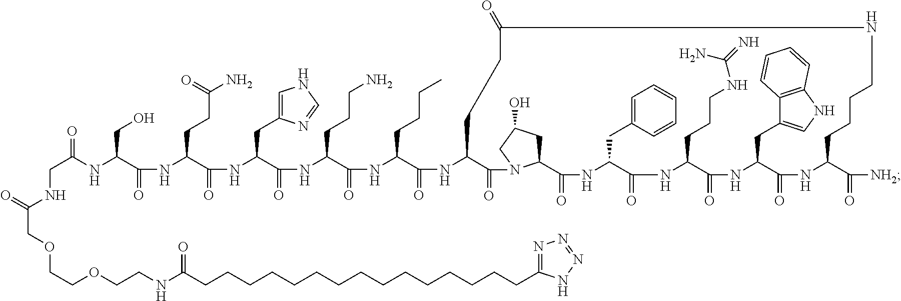
{2-[2-(16-(Tetrazol-5-yl)hexadecanoylamino)ethoxy]ethoxy}acetyl-Gly-Ser-Gln-His-Lys-Nle-c[Glu-Hyp-D-Phe-Arg-Trp-Lys]-NH₂ 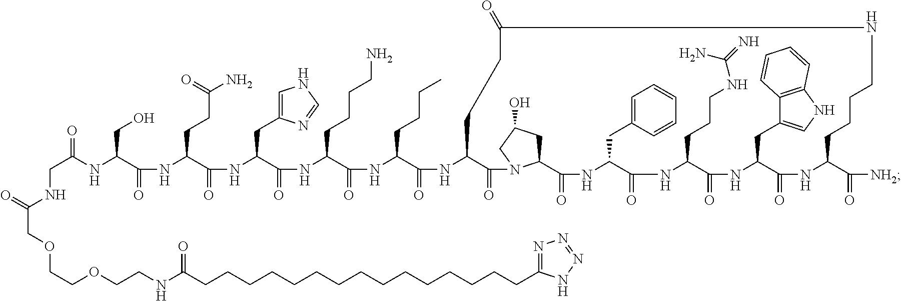
{2-[2-(16-(Tetrazol-5-yl)hexadecanoylamino)ethoxy]ethoxy}acetyl-Gly-Ser-Gln-His-Arg-Nle-c[Glu-Hyp-D-Phe-Arg-Trp-Lys]-NH₂ 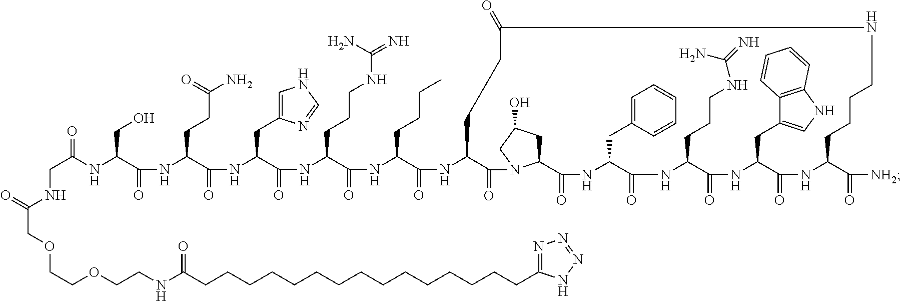
{2-[2-(16-(Tetrazol-5-yl)hexadecanoylamino)ethoxy]ethoxy}acetyl-Gly-Ser-Gln-His-2-PyAla-Nle-c[Glu-Hyp-D-Phe-Arg-Trp-Lys]-NH₂ 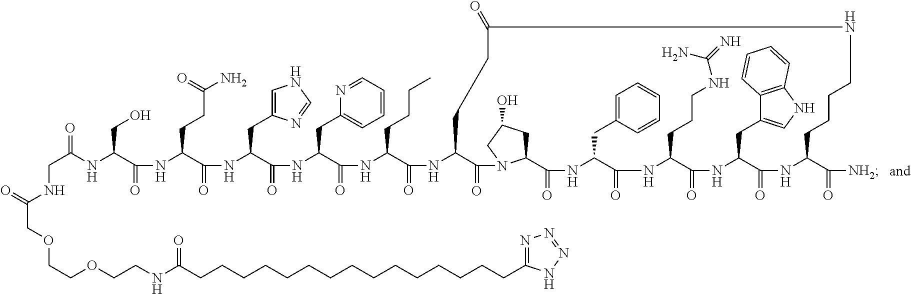
{2-[2-(16-(Tetrazol-5-yl)hexadecanoylamino)ethoxy]ethoxy}acetyl-Gly-Ser-Gln-His-4-PyAla-Nle-c[Glu-Hyp-D-Phe-Arg-Trp-Lys]-NH₂ [chemical structure]
